SEQUENCE LISTING (56 sequences)

SEQ ID NO: 1 - DNA, 1707 bp, Zymomonas mobilis
  source 1..1707: mol_type = other DNA
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 2 - DNA, 1707 bp, synthetic construct
  misc_feature 1..1707: note = Codon optimized sequence of SEQ ID NO: 1
  source 1..1707: mol_type = other DNA
atgtcatataccgttggcacctatttggctgaacgtttggttcaaatcggcttgaagcaccacttcgctgttgctggcgattataacttggttttgttggataacttgttgttgaacaagaacatggaacaagtttattgctgcaacgaattgaactgcggcttctcagctgaaggctatgctcgtgctaagggcgctgctgctgctgttgttacctattcagttggcgctttgtcagctttcgatgctatcggcggcgcttatgctgaaaacttgccagttatcttgatctcaggcgctccaaacaacaacgatcacgctgctggccacgttttgcaccacgctttgggcaagaccgattatcactatcaattggaaatggctaagaacatcaccgctgctgctgaagctatctataccccagaagaagctccagctaagatcgatcacgttatcaagaccgctttgcgtgaaaagaagccagtttatttggaaatcgcttgcaacatcgcttcaatgccatgcgctgctccaggcccagcttcagctttgttcaacgatgaagcttcagatgaagcttcattgaacgctgctgttgaagaaaccttgaagttcatcgctaaccgtgataaggttgctgttttggttggctcaaagttgcgtgctgctggcgctgaagaagctgctgttaagttcgctgatgctttgggcggcgctgttgctaccatggctgctgctaagtcattcttcccagaagaaaacccacactatatcggcacctcatggggcgaagtttcatatccaggcgttgaaaagaccatgaaggaagctgatgctgttatcgctttggctccagttttcaacgattattcaaccaccggctggaccgatatcccagatccaaagaagttggttttggctgaaccacgttcagttgttgttaacggcgttcgtttcccatcagttcacttgaaggattatttgacccgtttggctcaaaaggtttcaaagaagaccggcgctttggatttcttcaagtcattgaacgctggcgaattgaagaaggctgctccagctgatccatcagctccattggttaacgctgaaatcgctcgtcaagttgaagctttgttgaccccaaacaccaccgttatcgctgaaaccggcgattcatggttcaacgctcaacgtatgaagttgccaaacggcgctcgtgttgaatatgaaatgcaatggggccacatcggctggtcagttccagctgctttcggctatgctgttggcgctccagaacgtcgtaacatcttgatggttggcgatggctcattccaattgaccgctcaagaagttgctcaaatggttcgtttgaagttgccagttatcatcttcttgatcaacaactatggctataccatcgaagttatgatccacgatggcccatataacaacatcaagaactgggattatgctggcttgatggaagttttcaacggcaacggcggctatgattcaggcgctggcaagggcttgaaggctaagaccggcggcgaattggctgaagctatcaaggttgctttggctaacaccgatggcccaaccttgatcgaatgcttcatcggccgtgaagattgcaccgaagaattggttaagtggggcaagcgtgttgctgctgctaactcacgtaagccagttaacaagttgttgtag

SEQ ID NO: 3 - DNA, 1707 bp, synthetic construct
  misc_feature 1..1707: note = Codon optimized sequence of SEQ ID NO :1
  source 1..1707: mol_type = other DNA
atgagctacactgttggtacttacttagctgaacgcttagttcagatcggtttaaagcatcattttgctgttgcaggtgattacaacttagttttattagataacttattattaaacaagaatatggaacaggtttactgttgtaacgaattaaactgtggtttcagcgcagaaggttacgctcgcgctaagggtgctgctgcagcagttgttacttactcagttggtgctttaagcgctttcgacgctatcggtggtgcttacgctgaaaatttaccagttattttaatcagcggtgctcctaacaacaatgaccatgctgcaggtcatgttttacatcatgctttaggtaagactgattaccattaccaattagaaatggcaaagaacattactgctgctgcagaagctatttacactcctgaagaagcaccagctaaaattgatcatgttattaagactgctttacgcgaaaagaaacctgtttacttagaaattgcttgtaacatcgctagcatgccatgtgctgcaccaggtccagctagcgctttattcaacgacgaagctagcgacgaagcatcattaaacgctgcagttgaagaaactttaaagtttatcgcaaaccgtgacaaggttgcagttttagttggtagcaagttacgcgctgctggtgcagaagaagcagctgttaaattcgctgacgctttaggtggtgctgttgcaactatggctgcagctaagagcttcttccctgaagaaaatcctcattacatcggtacttcctggggtgaagtgagctacccaggtgttgaaaagactatgaaagaagcagatgcagttattgctttagctcctgtttttaatgattactcaactactggttggactgatatccctgatccaaagaaattagttttagctgaacctcgtagcgttgtcgttaacggtgttcgttttccaagcgttcatttaaaggattacttaactcgtttagctcaaaaagtttccaagaagactggtgctttagatttctttaagagcttaaacgctggtgaattaaagaaggcagctccagcagacccatctgctcctttagttaacgcagaaatcgcacgtcaggtcgaagctttattaactccaaacactactgttattgctgaaactggtgattcctggttcaatgctcaacgcatgaagttaccaaacggtgctcgcgttgaatacgaaatgcagtggggtcatatcgggtggtctgttccagcagcgttcggttacgctgttggtgctcctgaacgtcgcaacatcttaatggttggtgatggtagcttccagttaactgctcaggaagttgcacagatggttcgcttaaagttaccagttattatcttcttaatcaacaattacggttacactatcgaagtcatgatccatgatggtccatacaacaatatcaagaattgggactacgctggtttaatggaagtcttcaacggtaacggtggttacgatagcggtgctggtaagggtttaaaggcaaagactggtggtgaattagctgaagcaatcaaagttgctttagctaacactgatggtccaactttaatcgaatgtttcatcggtcgtgaagactgtactgaagaattagttaaatggggtaagcgcgttgctgcagcaaactcccgtaaaccagttaataagttattataa

SEQ ID NO: 4 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 5 - DNA, 1152 bp, Zymomonas mobilis
  source 1..1152: mol_type = other DNA
atggcttcttcaactttttatattcctttcgtcaacgaaatgggcgaaggttcgcttgaaaaagcaatcaaggatcttaacggcagcggctttaaaaatgcgctgatcgtttctgatgctttcatgaacaaatccggtgttgtgaagcaggttgctgacctgttgaaagcacagggtattaattctgctgtttatgatggcgttatgccgaacccgactgttaccgcagttctggaaggccttaagatcctgaaggataacaattcagacttcgtcatctccctcggtggtggttctccccatgactgcgccaaagccatcgctctggtcgcaaccaatggtggtgaagtcaaagactacgaaggtatcgacaaatctaagaaacctgccctgcctttgatgtcaatcaacacgacggctggtacggcttctgaaatgacgcgtttctgcatcatcactgatgaagtccgtcacgttaagatggccattgttgaccgtcacgttaccccgatggtttccgtcaacgatcctctgttgatggttggtatgccaaaaggcctgaccgccgccaccggtatggatgctctgacccacgcatttgaagcttattcttcaacggcagctactccgatcaccgatgcttgcgccttgaaggctgcgtccatgatcgctaagaatctgaagaccgcttgcgacaacggtaaggatatgccagctcgtgaagctatggcttatgcccaattcctcgctggtatggccttcaacaacgcttcgcttggttatgtccatgctatggctcaccagttgggcggctactacaacctgccgcatggtgtctgcaacgctgttctgcttccgcatgttctggcttataacgcctctgtcgttgctggtcgtctgaaagacgttggtgttgctatgggtctcgatatcgccaatctcggtgataaagaaggcgcagaagccaccattcaggctgttcgcgatctggctgcttccattggtattccagcaaatctgaccgagctgggtgctaagaaagaagatgtgccgcttcttgctgaccacgctctgaaagatgcttgtgctctgaccaacccgcgtcagggtgatcagaaagaagttgaagaactcttcctgagcgctttctaa

SEQ ID NO: 6 - DNA, 1152 bp, synthetic construct
  misc_feature 1..1152: note = Codon optimized sequence of SEQ ID NO: 5
  source 1..1152: mol_type = other DNA
atggcttcatcaaccttctatatcccattcgttaacgaaatgggcgaaggctcattggaaaaggctatcaaggatttgaacggctcaggcttcaagaacgctttgatcgtttcagatgctttcatgaacaagtcaggcgttgttaagcaagttgctgatttgttgaaggctcaaggcatcaactcagctgtttatgatggcgttatgccaaacccaaccgttaccgctgttttggaaggcttgaagatcttgaaggataacaactcagatttcgttatctcattgggcggcggctcaccacacgattgcgctaaggctatcgctttggttgctaccaacggcggcgaagttaaggattatgaaggcatcgataagtcaaagaagccagctttgccattgatgtcaatcaacaccaccgctggcaccgcttcagaaatgacccgtttctgcatcatcaccgatgaagttcgtcacgttaagatggctatcgttgatcgtcacgttaccccaatggtttcagttaacgatccattgttgatggttggcatgccaaagggcttgaccgctgctaccggcatggatgctttgacccacgctttcgaagcttattcatcaaccgctgctaccccaatcaccgatgcttgcgctttgaaggctgcttcaatgatcgctaagaacttgaagaccgcttgcgataacggcaaggatatgccagctcgtgaagctatggcttatgctcaattcttggctggcatggctttcaacaacgcttcattgggctatgttcacgctatggctcaccaattgggcggctattataacttgccacacggcgtttgcaacgctgttttgttgccacacgttttggcttataacgcttcagttgttgctggccgtttgaaggatgttggcgttgctatgggcttggatatcgctaacttgggcgataaggaaggcgctgaagctaccatccaagctgttcgtgatttggctgcttcaatcggcatcccagctaacttgaccgaattgggcgctaagaaggaagatgttccattgttggctgatcacgctttgaaggatgcttgcgctttgaccaacccacgtcaaggcgatcaaaaggaagttgaagaattgttcttgtcagctttctaa

SEQ ID NO: 7 - DNA, 1152 bp, synthetic construct
  misc_feature 1..1152: note = Codon optimized sequence of SEQ ID NO: 5
  source 1..1152: mol_type = other DNA
atggctagcagcactttctacatccctttcgttaatgaaatgggtgaaggttcattagaaaaggcaattaaggatttaaacggttcaggttttaagaacgcattaatcgttagcgatgcattcatgaataagagcggtgttgttaaacaggttgctgacttattaaaggcacaaggtatcaacagcgctgtttacgatggtgttatgcctaacccaactgttactgctgttttagaaggtttaaagattttaaaggacaacaacagcgacttcgttatttcattaggtggtggttcaccacatgattgtgctaaggcaatcgcattagttgcaactaacggtggtgaagttaaagattacgaaggtatcgacaagagcaagaagcctgctttaccattaatgagcatcaacactactgctggtactgctagcgaaatgactcgtttctgtatcatcactgacgaagttcgccatgttaaaatggcaattgttgaccgtcatgttactcctatggttagcgttaacgacccattattaatggttggtatgcctaagggtttaactgctgctactggtatggacgctttaactcatgcattcgaagcatactcatcaactgctgcaactccaattactgatgcttgtgctttaaaggcagctagcatgatcgcaaagaacttaaagactgcttgtgataacggtaaggacatgcctgcacgtgaagcaatggcttacgctcagttcttagctggtatggcattcaataacgctagcttaggttacgttcatgcaatggcacatcagttaggtggttactacaacttaccacatggtgtttgcaatgctgtgctgttaccacatgttttagcttacaacgctagcgttgttgcaggtcgtttaaaggacgttggtgttgcaatgggtttagacatcgcaaacttaggtgacaaggaaggtgctgaagcaactatccaggcagttcgtgacttagctgctagcatcggtatccctgctaacttaactgaattaggtgcaaagaaggaagacgttcctttattagctgaccatgctttaaaggacgcttgtgctttaactaaccctcgtcaaggtgatcagaaagaagtcgaagaattattcttaagcgcattctaa

SEQ ID NO: 8 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 9 - protein, 495 aa, Saccharomyces cerevisiae
MTTDNAKAQLTSSSGGNIIVVSNRLPVTITKNSSTGQYEYAMSSGGLVTALEGLKKTYTFKWFGWPGLEIPDDEKDQVRKDLLEKFNAVPIFLSDEIADLHYNGFSNSILWPLFHYHPGEINFDENAWLAYNEANQTFTNEIAKTMNHNDLIWVHDYHLMLVPEMLRVKIHEKQLQNVKVGWFLHTPFPSSEIYRILPVRQEILKGVLSCDLVGFHTYDYARHFLSSVQRVLNVNTLPNGVEYQGRFVNVGAFPIGIDVDKFTDGLKKESVQKRIQQLKETFKGCKIIVGVDRLDYIKGVPQKLHAMEVFLNEHPEWRGKVVLVQVAVPSRGDVEEYQYLRSVVNELVGRINGQFGTVEFVPIHFMHKSIPFEELISLYAVSDVCLVSSTRDGMNLVSYEYIACQEEKKGSLILSEFTGAAQSLNGAIIVNPWNTDDLSDAINEALTLPDVKKEVNWEKLYKYISKYTSAFWGENFVHELYSTSSSSTSSSATKN

SEQ ID NO: 10 - protein, 896 aa, Saccharomyces cerevisiae
MTTTAQDNSPKKRQRIINCVTQLPYKIQLGESNDDWKISATTGNSALYSSLEYLQFDSTEYEQHVVGWTGEITRTERNLFTREAKEKPQDLDDDPLYLTKEQINGLTTTLQDHMKSDKEAKTDTTQTAPVTNNVHPVWLLRKNQSRWRNYAEKVIWPTFHYILNPSNEGEQEKNWWYDYVKFNEAYAQKIGEVYRKGDIIWIHDYYLLLLPQLLRMKFNDESIIIGYFHHAPWPSNEYFRCLPRRKQILDGLVGANRICFQNESFSRHFVSSCKRLLDATAKKSKNSSDSDQYQVSVYGGDVLVDSLPIGVNTTQILKDAFTKDIDSKVLSIKQAYQNKKIIIGRDRLDSVRGVVQKLRAFETFLAMYPEWRDQVVLIQVSSPTANRNSPQTIRLEQQVNELVNSINSEYGNLNFSPVQHYYMRIPKDVYLSLLRVADLCLITSVRDGMNTTALEYVTVKSHMSNFLCYGNPLILSEFSGSSNVLKDAIVVNPWDSVAVAKSINMALKLDKEEKSNLESKLWKEVPTIQDWTNKFLSSLKEQASSDDDVERKMTPALNRPVLLENYKQAKRRLFLFDYDGTLTPIVKDPAAAIPSARLYTILQKLCADPHNQIWIISGRDQKFLNKWLGGKLPQLGLSAEHGCFMKDVSCQDWVNLTEKVDMSWQVRVNEVMEEFTTRTPGSFIERKKVALTWHYRRTVPELGEFHAKELKEKLLSFTDDFDLEVMDGKANIEVRPRFVNKGEIVKRLVWHQHGKPQDMLKGISEKLPKDEMPDFVLCLGDDFTDEDMFRQLNTIETCWKEKYPDQKNQWGNYGFYPVTVGSASKKTVAKAHLTDPQQVLETLGLLVGDVSLFQSAGTVDLDSRGHVKNSESSLKSKLASKAYVMKRSASYTGAKV

SEQ ID NO: 11 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 12 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 13 - protein, 292 aa, Bifidobacterium adoloscentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 14 - protein, 791 aa, Bifidobacterium adoloscentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 15 - protein, 910 aa, Bifidobacterium adoloscentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 16 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 17 - protein, 510 aa, Lactobacillus paracasei
MVKNTLNRDIPEPYADQYGVYGGEFANIKPYDEHARHINPVKPDHSKLVASIHDAIVATGLKDGMTISFHHHFREGDYVMNMVLAEIAKMGIKNLSIAPSSIANVHEPLIEHIKNGVVTNITSSGLRDKVGAAISSGIMKNPVVIRSHGGRARAIARGDIHIDVAFLGAPSSDEYGNINGTKGKATCGSLGYAMIDAKYADQVVAITDSLMPYPNTPISIPQTDVDYVVQVDAIGDPTGIAKGATRFTKNPKELKIAEYAAEVITKSAYFKNGFSFQTGTGGSSLAVARFLRQAMLDQDIKASFALGGITNSMVELLKEGLVEKIIDVQDFDHPSAVSLGENADHYEIDANMYASPLSKGAVINQLDIAILSALEIDTNFNVNVITGSDGIIRGASGGHSDTSAACKMSMVIAPLVRGRIPTIVENVNTVVTPGASVDVVVTEVGVAINPARTDLIEMFKNLKVPLFSIEDLKKMAYQITGTPEAIEYGDKVVALIEYRDGTLIDVVHNV

SEQ ID NO: 18 - DNA, 1533 bp, Lactobacillus paracasei
  source 1..1533: mol_type = other DNA
atggtcaagaatacactcaaccgtgatatcccagaaccatatgcggatcaatacggtgtttatggcggcgagtttgccaacattaagccttatgatgaacatgcccgccacatcaatccggttaagccggatcacagcaaactcgtggcgtcaattcacgatgccattgtagcaactgggctgaaggacggcatgaccatttcttttcaccatcattttcgtgaaggggactatgtgatgaacatggtcctagctgagattgccaaaatggggatcaagaacctgtcaattgcgccaagttcgattgccaatgtacatgaaccattgattgagcacatcaaaaacggtgtggtgaccaacatcaccagttccggcttgcgcgacaaagtgggggcagcaatttcaagcggcatcatgaagaatccagttgtgattcgctcacatggcggccgggcccgagccattgctcgtggcgatattcatattgacgttgcctttcttggtgcccctagcagtgatgagtacggcaacattaacggcacaaaaggtaaggcgacctgtggctcgttaggttatgccatgattgacgcaaaatatgcggatcaagttgttgccattactgacagtttaatgccatatccgaatacgccaatcagcattccgcaaaccgacgttgactatgtcgtgcaagttgatgcgattggcgatccaactgggattgccaaaggtgcgacccgtttcacgaagaacccgaaggaattaaaaattgcggagtatgcggcagaggtcattaccaaatcggcctacttcaaaaatgggttctcattccagaccggtactggcggctcttcgctggctgttgcgcgttttctgcggcaagcgatgttggaccaagacatcaaagctagttttgctttgggtggcattaccaattcaatggttgaattgttgaaggaaggccttgtcgaaaagattatcgatgtgcaggactttgaccatccctctgcggtttcattaggcgagaacgcagatcattacgagattgatgctaatatgtacgcgtcaccgttaagcaaaggtgcagttatcaatcagttagatattgcgattttatcggcactggaaattgatactaactttaacgttaacgtgatcacgggttctgacggcattatccgtggcgcttctggtggccatagtgacacaagtgctgcctgcaaaatgagcatggtgattgcgccactggttcgcggtcggatcccaacgattgttgaaaatgtcaatactgttgtgacaccgggtgccagtgttgacgttgtcgtgaccgaagtcggcgtcgctattaatccagcacggactgatttgattgaaatgtttaaaaatctgaaagtgccgctgttttcaattgaagatctgaaaaagatggcctatcaaattactggtacaccggaagccatcgaatatggcgataaagtggtcgctttgatcgaatatcgcgatggcaccttgatcgatgtggttcacaatgtttaa

SEQ ID NO: 19 - protein, 292 aa, Lactobacillus paracasei
MDKLRRTMMFVPGANPGMLRDAPIYGADAIMFDLEDAVSLKEKDTARMLVYSALKTFDYSSVETVVRVNALDAGGDQDIEAMVLGGINVVRLPKTETAQDIIDVDAVITAVEEKYGIQNGTTHMMAAIESAEGVLNAREIAQASSRMIGIALGAEDYLTSQHTHRSTDGAELSFARNYILHAAREAGIAAIDTVYTQVDNEEGLRHETALIKQLGFDGKSVINPRQIPVINGVFAPALAEVQKAREIVAGLKEAEAKGAGVVSVNGQMVDKPVVERAQYTIALAKASGMEVD

SEQ ID NO: 20 - DNA, 879 bp, Lactobacillus paracasei
  source 1..879: mol_type = other DNA
atggataaattaagaagaaccatgatgtttgtgcctggtgccaatccgggcatgttacgtgatgctccgatttatggtgctgatgcgatcatgtttgaccttgaagatgctgtttctttgaaggaaaaagacacggcgcgaatgctggtttattcagccctgaagacctttgattacagtagcgtggaaacagttgtgcgggtgaatgcgttagatgcaggcggggatcaagacattgaagcgatggttcttggcggcattaatgtggtgcgcctgccaaagaccgaaactgctcaagacattattgatgttgatgctgtcatcacagcagttgaagagaagtacggcattcaaaatggcaccacgcacatgatggctgcaattgagtcggctgaaggggttttgaatgctcgcgaaatcgcacaagcttcatcacgtatgattgggattgcgttgggtgcagaagattatctgacgagtcaacatacccaccgctcgacggatggcgctgaattgtcttttgcccgtaactatatcctgcatgctgcgcgagaagctggcatcgcggcgattgatacggtctatacacaagtggacaacgaagaaggtttgcgccacgaaaccgccttaatcaaacagcttggctttgatggcaagtccgtcatcaatccacggcaaattccagtcattaatggggtttttgcccctgctttggcggaagttcaaaaagcacgtgagattgttgctggcttgaaagaagccgaagctaagggcgcgggggttgtttctgtgaacgggcagatggttgataagccagttgtcgaacgggcacagtataccatcgcgctcgcaaaggcatcaggaatggaggtagactga

SEQ ID NO: 21 - protein, 101 aa, Lactobacillus paracasei
MEIKHPATAGTLESSDIQITLSPATSGVAIQLQSSVEKQFGHQIRSVIEATLAKLGIENVAVDANDKGALDCTIKARTIAAVYRASDNKTFDWEEINAWIN

SEQ ID NO: 22 - DNA, 306 bp, Lactobacillus paracasei
  source 1..306: mol_type = other DNA
atggaaattaagcatcctgcgactgctggtacgctggaatcaagtgatattcaaatcaccttgtcaccggctaccagtggggtcgccattcaactgcaaagcagtgtagaaaaacagtttggtcatcaaattcgatcagtcattgaggccaccttggccaagttaggaatcgaaaatgttgccgttgacgcgaatgacaaaggcgccttggattgtaccatcaaggcgcggacgatcgccgctgtttatcgtgcgtctgacaataagacgtttgactgggaggagatcaacgcatggataaattaa

SEQ ID NO: 23 - protein, 467 aa, Lactobacillus paracasei
MPKQKVQFMETVLRDGQQSLIATRMPLSDILPILDKMDAAGYASLEMWGGATFDACLRYLNEDPWERLRKIRKAVKHTKLQMLLRGQNLLGYKNYADDVVTDFVTKSVENGIDIIRIFDALNDTRNLRTALEATKQAGGHAQLAICYTTSDFHTIDYFIKLAKDMADMGADSIAIKDMAGILTPQKAFDLVTGIKQEISVPLEVHTHATAGMAEMTYLEAVRAGADIIDTAVSPFAGGTSQPATESMLVALQDLGYPTDVDLSTVSDIATYFAPIRDRFRESGQLNPRVKDVEPKSLIYQVPGGMLSNLLAQLKDQGQEALYGDVLKEVPRVRADLGYPPLVTPLSQMVGTQSLMNVMSGERYKLIPNEIKDYVRGLYGRPPVAIAPEMVKKIIGDAPVVTQRPADLIKPQMPDFRQAIAQYAHDEEDVLSYALFPDQAKDFLGRREDPFYDVPEQKVSLSFEPTHD

SEQ ID NO: 24 - DNA, 1404 bp, Lactobacillus paracasei
  source 1..1404: mol_type = other DNA
atgcctaaacagaaagtccaattcatggaaaccgttttgcgtgacgggcaacaaagcctgattgccacgcggatgccgctcagcgatattttgccgattctcgataaaatggatgctgctggctatgcatctttggaaatgtggggcggggcaacttttgatgcctgtctccgttatctgaatgaagatccgtgggaacggttgcgcaagattcgtaaggcggtcaagcacaccaaattgcaaatgctcttgcgcgggcaaaacttgttaggttacaaaaactatgccgacgatgtggtcactgactttgtcacaaagtcagttgaaaacgggattgatatcattcgtattttcgatgcgttgaatgatacacgcaacttgcgaacggcgcttgaagcgacgaagcaagcaggcgggcatgctcaacttgctatttgttacacaaccagtgatttccatacgatcgactacttcatcaagttagccaaagacatggctgacatgggtgcggattcaattgcaatcaaagacatggcaggcattttaaccccacagaaggcgtttgatctggttaccggtattaagcaggaaatcagcgtgccactggaagtgcatacgcacgccaccgctggtatggctgagatgacgtatctggaagcagttcgcgccggtgctgatatcattgataccgcggtttcgccatttgctggcggcaccagtcagccagcaacagaatccatgctggttgcgttgcaagatcttggctatccgactgatgttgatttaagcacggtcagtgacatcgccacttactttgcgccgattcgcgatcgattccgcgagtccggtcaactgaatccgcgcgtgaaagatgttgaacctaaatccttgatctatcaggtgccaggcgggatgttgtctaacctgttggcgcaactaaaagatcaaggacaagaagcgctttatggcgatgttttgaaggaagtgccgcgtgtccgagctgacttaggctatccgccgttggttacaccgctgtcgcagatggtgggcacacaaagtttgatgaatgtcatgagcggtgagcgttataagttgattccaaatgaaattaaggattacgtgcgcggcctttatggtcggccgccagtggcaattgcacccgaaatggtgaaaaagatcattggtgatgcaccggttgtcacacaacgtcccgcggatttaatcaagccgcaaatgcctgatttccgtcaagcgattgcgcaatatgcgcacgacgaagaggatgtcttaagctatgctttgttcccagatcaagctaaagattttcttggccggcgcgaagatccgttttatgatgtgccggagcagaaggtgtcgctaagttttgagccgacgcatgattga

SEQ ID NO: 25 - protein, 374 aa, Lactobacillus paracasei
MEALIHGITTITLGQIAMMLIGALLMYLGIKKEYEPTLLVPMGLGAILVNFPGTGVLTQVVGGTKAEGVLDVLFKAGINTELFPLLIFIGIGAMIDFGPLLQNPFMLLFGAAAQFGIFATVFVAVFFGFNIKEAASIGIIGAADGPTSIFVSNQLAPNLLGAITVAAYSYMALVPIIQPMAIKAVTTKHERRIRMTYKAEGVSKTTKILFPIIITIIAGFIAPISLPLVGFLMFGNLLRECGVLDRLSNTAQNELVNIVSILLGLTISVKLQADQFLNIQTLMIIAFGLFAFIMDSVGGVLFAKLLNLFRKDKINPMIGAAGISAFPMSSRVIQKMATDEDPQNFVLMYAVGANVSGQIGSVIAGGLLLSFFGA

SEQ ID NO: 26 - DNA, 1125 bp, Lactobacillus paracasei
  source 1..1125: mol_type = other DNA
atggaagcgctcattcacggaatcaccacgatcacattaggtcaaatcgccatgatgctgatcggcgcgctcctgatgtatctgggaatcaaaaaagaatatgaaccaacccttttagttcccatgggcttgggcgccattctggtcaactttccaggaacaggcgtcttgacccaagttgttggcggcaccaaagcagaaggggtgcttgatgttttattcaaagccggtatcaatacggaactgttcccactgctaattttcatcgggatcggcgccatgatcgattttggaccgttattacaaaacccatttatgctactgttcggtgcagcagcacagttcgggatctttgccaccgtttttgttgctgtcttcttcggtttcaatatcaaagaagcggcttcaattggtatcatcggtgccgcagacggcccgacttcgattttcgtttcgaaccaacttgcgccaaatctgttaggggccatcacagtcgctgcgtattcgtatatggcattggtgccgatcatccagccaatggccatcaaggcagtgaccacaaagcatgaacgccggattcggatgacttataaggcagaaggcgtttcaaagacgacaaaaattctgtttccaatcattatcacgattattgccgggtttattgccccgatttccttaccgttagttgggttcctgatgtttggtaacctgctgcgagaatgtggggtgctcgatcggctgtctaacaccgcgcaaaacgaattggtcaatattgtgtcgattctgcttgggctaacgatttccgttaaattgcaagctgatcaattcttgaacattcaaacgttgatgatcattgcttttggattattcgccttcatcatggactctgtcgggggtgtattgttcgccaaattattgaatcttttccgtaaagataagattaacccaatgatcggggcggccggcatttccgctttcccaatgtcgagccgagtgattcaaaaaatggcaaccgatgaagatccacagaattttgttttgatgtatgccgttggcgctaatgtttccggccaaatcggttctgtcattgccggcggactgttactatcattctttggcgcataa

SEQ ID NO: 27 - protein, 382 aa, Escherichia coli
MKALHFGAGNIGRGFIGKLLADAGIQLTFADVNQVVLDALNARHSYQVHVVGETEQVDTVSGVNAVSSIGDDVVDLIAQVDLVTTAVGPVVLERIAPAIAKGQVKRKEQGNESPLNIIACENMVRGTTQLKGHVMNALPEDAKAWVEEHVGFVDSAVDRIVPPSASATNDPLEVTVETFSEWIVDKTQFKGALPNIPGMELTDNLMAFVERKLFTLNTGHAITAYLGKLAGHQTIRDAILDEKIRAVVKGAMEESGAVLIKRYGFDADKHAAYIQKILGRFENPYLKDDVERVGRQPLRKLSAGDRLIKPLLGTLEYGLPHKNLIEGIAAAMHFRSEDDPQAQELAALIADKGPQAALAQISGLDANSEVVSEAVTAYKAMQ

SEQ ID NO: 28 - DNA, 1149 bp, synthetic construct
  misc_feature 1..1149: note = Encoding SEQ ID NO: 27 and codon-optimized for expression in Saccharomyces cerevisiae
  source 1..1149: mol_type = other DNA
atgaaggcactgcacttcggggctggcaacataggtcgtggctttataggcaagttactagctgacgccggtattcaactaacctttgcagatgtaaatcaggttgtcctagacgccctgaatgcaaggcatagttatcaagtccatgtagttggcgaaacggaacaggttgatacggtgtccggagtgaatgcagtgtcttctataggcgatgacgtggtcgatctgattgcacaagttgacttggtcaccactgcggtaggaccagtcgtcttagaacgtatagctcctgcaatcgccaagggtcaggtcaagaggaaggagcagggcaacgagagccccctgaatatcattgcttgcgaaaacatggttagggggaccactcagttgaaaggccacgtaatgaacgcattgccagaggatgcgaaggcctgggtagaagagcatgtcggttttgtcgattcagctgttgacagaatcgtgcccccgtccgcttctgctactaacgacccgcttgaggtcacagtagaaactttcagcgaatggatcgtagacaaaacacaatttaagggcgccctgcctaacataccgggtatggaactaacagacaatttaatggcattcgtggagagaaaattatttactcttaacacaggccatgccatcaccgcctatttagggaaattagcgggccatcagactataagagatgcgattctagacgaaaaaatccgtgccgtcgtgaaaggtgccatggaagaaagtggcgccgtcctgattaagcgttacggttttgatgcagataagcatgccgcgtatattcagaaaatcctgggccgtttcgaaaatccatatttgaaggacgatgtggagcgtgtgggtcgtcagccgttgaggaagttatctgctggtgaccgtctaattaagcctctgctaggcactttggagtacggactgccacataagaacctgatagaggggattgcagctgcaatgcatttcaggagcgaagatgaccctcaggcacaagagttggctgctctgattgcagacaaaggtcctcaagccgctttggcgcagatctcaggcctagatgctaatagtgaggttgtcagtgaggccgtaacggcctataaggcaatgcaataa

SEQ ID NO: 29 - protein, 259 aa, Escherichia coli
MNQVAVVIGGGQTLGAFLCHGLAAEGYRVAVVDIQSDKAANVAQEINAEYGESMAYGFGADATSEQSVLALSRGVDEIFGRVDLLVYSAGIAKAAFISDFQLGDFDRSLQVNLVGYFLCAREFSRLMIRDGIQGRIIQINSKSGKVGSKHNSGYSAAKFGGVGLTQSLALDLAEYGITVHSLMLGNLLKSPMFQSLLPQYATKLGIKPDQVEQYYIDKVPLKRGCDYQDVLNMLLFYASPKASYCTGQSINVTGGQVMF

SEQ ID NO: 30 - DNA, 780 bp, synthetic construct
  misc_feature 1..780: note = Encoding SEQ ID NO: 29 and codon-optimized for expression in Saccharomyces cerevisiae
  source 1..780: mol_type = other DNA
atgaaccaggtggcagttgtgatcggcggcggccagacacttggagcgttcctttgtcacggcttagcagccgagggttacagggtagccgtcgtagacattcagtcagataaagcagccaacgtcgctcaagagataaatgcggaatacggggagtcaatggcctacggatttggtgctgatgcaactagcgaacagtctgttcttgctctttcaaggggggtagatgaaattttcggacgtgttgatctgcttgtctattcagccggaatcgcgaaagctgccttcatttctgattttcaattaggtgattttgacaggtcccttcaagtcaatttagtaggttattttttatgtgctagggagttttccagacttatgattagggatgggattcagggccgtataatccagatcaacagtaagagtggtaaggtcggcagcaaacacaatagcgggtattccgccgcgaagtttggaggtgtcggtcttacacaatcccttgccctagatctagccgaatatggcattacagtacactctctgatgctgggcaatttactgaaatcaccgatgtttcaaagcttactgccacagtacgcgacgaaactaggcatcaaacccgaccaggtcgaacagtattatattgataaagttcctttgaagaggggatgcgactatcaagatgtgcttaatatgttgttgttttacgcctcacccaaggcgtcttattgcacaggccaatctatcaatgtaactggtggacaagtcatgttctaa

SEQ ID NO: 31 - protein, 266 aa, Lactobacillus paracasei
MSDWLGLDGKTIVVTGGSSGIGAAIVKELINNGATVVNGDLKEGDFKDPNLKYVHTDVTDPDEVENLAATAEKINGEIWGVVNNAGINKPRVLVDPKDPHGKYELDVKTFEQIFSVNVKSVFLVSQAVVRRMVKQGHGVVVNMSSEAGLEGSVGQSVYSASKGAINGFTRSWAKELGKYNIRVVGVAPGIMEATGLRTPSYEEALAYTRDTTVDAIRAGYSSTSTTPLGRSGKLSEVGDLVNYFLSNRASYITGVTTNVAGGKSRG

SEQ ID NO: 32 - DNA, 801 bp, synthetic construct
  misc_feature 1..801: note = Encoding SEQ ID NO: 31 and codon-optimized for expression in Saccharomyces cerevisiae
  source 1..801: mol_type = other DNA
atgagtgattggcttggtctggatggcaaaaccatcgtcgtcactggtggttcctctggcatcggtgcggctatcgttaaagaattgattaataacggcgcgactgttgttaacggcgacttgaaagaaggggacttcaaagatccgaatcttaagtatgttcacacggacgttacagatcccgatgaagttgagaaccttgctgcaaccgctgaaaagatcaatggcgaaatttggggtgttgttaacaacgctggcatcaacaagcctcgtgttttggtagatcctaaggacccacatggcaagtatgaattggatgtgaagacattcgaacagattttctcagttaacgttaaatcagttttccttgtttcccaggccgtggtccgccgtatggttaagcaaggccatggcgttgttgttaacatgtcatccgaagcaggtcttgagggcagcgttggtcaatcagtgtactcagcatcaaagggcgcaatcaatggctttacacggtcctgggctaaggaacttggtaaatacaacattcgtgtcgttggcgtcgctccaggtatcatggaagctacgggcttgcgtacgccaagctacgaagaagctttggcttacacccgtgatactaccgtagacgctattcgtgctggctactcaagtaccagcaccactcctctcggtcgctcaggtaagttgtctgaagttggggacctggttaattattttctatcaaaccgtgcctcctatattactggcgttacaactaatgttgctggcgggaaatctcgtggctaa

SEQ ID NO: 33 - protein, 189 aa, Lactobacillus paracasei
MQYVSDFAAGFMKLFQTGGKTFISWMTSIVPVVLLLLVLMNTIIAFIGEERIERFAQKASRNVLMRYLVLPFLAAFMLGNPMCFTLARFLPEYYKPSYYAAQAQFCHTSNGVFPHINPGELFVWLGIAQGVQKLGLNQMDLAIRYMLVGIVMNFIGGWVTDFTTAYVSKQTGITLSKTVDLSARNGQEA

SEQ ID NO: 34 - DNA, 570 bp, synthetic construct
  misc_feature 1..570: note = Encoding SEQ ID NO: 33 and codon-optimized to prevent homologous recombination with native host gene
  source 1..570: mol_type = other DNA
atgcagtatgtttctgactttgcagcaggcttcatgaaattgttccagaccgggggcaagacttttatctcatggatgacttcgatcgtcccagtggtcctgttgctcctggttttgatgaacactattatcgctttcatcggcgaagaacggatcgaacgttttgcacaaaaggctagccgtaatgtcctgatgcggtacctggttttaccatttttggcagctttcatgctgggtaatccgatgtgtttcaccttggctcgcttcctcccagaatactataagccatcctattacgccgctcaagcccaattttgccacacctcaaacggcgttttcccgcacattaatcctggcgaactgttcgtttggctgggtattgctcaaggtgtccaaaaattgggcttgaaccaaatggatttggccatccggtatatgttagttggcatcgttatgaacttcatcggtggttgggttaccgactttacaactgcatatgtttcaaagcaaacgggtatcaccttgtccaaaactgtggatttatccgcacgtaatggtcaggaagcttaa

SEQ ID NO: 35 - protein, 372 aa, Lactobacillus paracasei
MADQKWHSIQVVKGSGGYGGPLTITPTEQKHKFIYVTGGNRPAIVDKIVELTGMEAVDGFKTSIPEDETAVAIIDCGGTLRCGIYPKKNILTINVLPTGKSGPLAKYIVPKLYVSNVDVNQITALPDDAVPDQSLNGVPFDQRGEAGKQHAALAESAASQATATEAKTTAAKDQEAAEARETKFDTNKTITAQMKKPNFIARIGIGAGKVIATFNQAAKDSVQTMLNTVIPFMAFVALLIGIIQGSGLGSWFAKLMTPLAGNVFGLIVIGFICSLPFLSPILGPGAVIAQVIGTLIGVEIGRGNIQPQYALPALFAINTQNAADFIPVGLGLEEADSKTVEVGVVSVLYSRFLNGVPRVVVAWLASFGLYAK

SEQ ID NO: 36 - DNA, 1119 bp, synthetic construct
  misc_feature 1..1119: note = Encoding SEQ ID NO: 35 and codon-optimized to prevent homologous recombination with the native host gene
  source 1..1119: mol_type = other DNA
atggctgatcaaaagtggcactccattcaagtggtcaagggtagcggtggttacggtggtccattgaccatcactccaactgaacagaagcacaagtttatttacgtgacggggggtaatcgtcctgcgattgttgacaagatcgttgaactgaccggtatggaagctgtggatggtttcaaaacttctattccagaagatgaaacggctgtcgctattatcgactgcggcggtaccctgcggtgcggtatttacccaaaaaagaacattcttacgattaacgttctcccgactggtaagtccggccctcttgctaagtatatcgttccgaagttgtatgtgtccaacgttgatgttaatcagattaccgctctaccagacgatgctgtaccagaccaatcattgaacggtgtccccttcgatcagcggggtgaagctggcaaacaacacgccgctctggcagaaagtgctgcttcccaagctaccgctaccgaggccaaaaccactgctgcgaaggatcaggaagctgctgaagctcgcgaaacgaaattcgacaccaacaagacaatcaccgcacaaatgaagaaacctaacttcattgcacgcatcggtatcggcgctggcaaggtcattgcaaccttcaaccaagcggctaaagactctgtccaaaccatgctgaacaccgttattccatttatggccttcgttgctcttttaatcggtattatccagggctcaggtctgggctcatggtttgctaaacttatgactccattagccggcaacgtattcggtttgattgttatcggtttcatttgctccttgccattcttgtctccaatcctcggcccgggggctgttattgcacaagtcattggtaccctgatcggcgttgagatcggtcgtggtaacatccaaccgcaatacgctttaccagcactctttgcaattaacacgcagaatgccgctgattttattcctgttggtttagggttggaagaagcagattccaagaccgttgaagtcggtgttgttagtgttttgtactcccgtttcctgaatggcgttccacgcgttgttgtcgcttggttggcttccttcggcctgtacgccaagtaa

SEQ ID NO: 37 - protein, 126 aa, Lactobacillus paracasei
MSSLAETVKYETKILEVGSEARGFKDINMAILFGDEAPDALRSSCFIINVNKILEPIEVGDVMTFDDQSYKITAVGNEVNTNLGNLGHTAIVFDGSTTPELAGSLYLEEKTYPELDVGTTIKIIRA

SEQ ID NO: 38 - DNA, 381 bp, synthetic construct
  misc_feature 1..381: note = Encoding SEQ ID NO: 37 and codon-optimized to prevent homologous recombination with native host gene
  source 1..381: mol_type = other DNA
atgtcgtccttggctgagaccgttaaatacgaaacaaagattttggaagttggcagcgaagctcgcggcttcaaagatatcaatatggcaattctgttcggtgatgaagctccggacgcccttcgttcctcatgcttcatcatcaacgttaataagattctggaaccaattgaagttggggatgtcatgacctttgatgatcaaagctacaaaatcaccgctgttggcaatgaagttaacactaacttgggtaacctgggccataccgctatcgtttttgacgggtccacgactccggaattggcgggctccttgtacttggaagaaaagacgtatccagaactcgatgttggcactaccatcaaaattattcgggcttga

SEQ ID NO: 39 - protein, 384 aa, Lactobacillus paracasei
MMEAVHFGAGNIGRGFIGETLAANGFKINFVDVNETIINALNQRGEYTITLAAPGEKKIHVDNVDGLNNAKDPEAVVKAIAQADLVTTAIGPKILPIIAPLIAQGLQARDAANNHQALDVIACENMIGGSQSLKKSVYEHLDDAGKTFADTYVGFPNAAVDRIVPQQKHDDPLAVSVEDFKEWVVDESQMKNKDLKLKTVDYVPDLEPYIERKLFSVNTGHATTAYTGKYLGYTTIGDAIKDPKVFNQAKGALAETRSLLLSEFKNFDEKDLENYQNRVLQRFQNPYISDDISRVARTPIRKLGYDERFIRPIRELKERGLNYSVLMDTVGMMFHYVEPNDAEAVKLQAMLKDQPLVDVIKEVTGLKDAGLIDEVEASVKSKDR

SEQ ID NO: 40 - DNA, 1155 bp, synthetic construct
  misc_feature 1..1155: note = Encoding SEQ ID NO: 39 and codon-optimized to prevent homologous recombination with native host gene
  source 1..1155: mol_type = other DNA
atgatggaagcagttcattttggtgcaggtaacatcggccggggcttcattggtgaaacattggccgctaacggcttcaaaattaatttcgtcgacgtgaatgaaacgattatcaatgctttaaaccagcgcggcgaatacactatcactctcgcggcacctggcgaaaagaagattcacgttgacaacgtggacggtctgaataacgcgaaagacccggaagcagtcgttaaggcaattgcccaagctgatcttgtgaccaccgccatcggcccaaagattcttccaattatcgcaccacttattgctcaaggtttgcaagcacgtgatgctgcgaacaaccatcaagccttggacgtgatcgcttgcgaaaacatgatcggaggctcacaatcccttaagaagagtgtctatgaacatttggatgatgctggtaaaaccttcgccgatacctatgtcggtttccctaatgccgctgtggatcgtattgtcccgcaacaaaagcatgatgacccacttgcggtcagtgttgaagatttcaaggaatgggtggtcgatgaaagtcaaatgaagaataaggatttaaagttgaagactgttgactacgttcctgacctcgagccttacattgaacgtaagttgttttccgttaatactggtcatgcaaccactgcgtatacaggtaaataccttggctacacaacgatcggtgacgcaattaaggatcctaaggtttttaaccaagccaagggtgcgctggccgaaacacgtagtctgttactttcagaattcaaaaactttgatgaaaaagacttggaaaactaccaaaaccgcgtcttgcaacggtttcaaaacccatatatctccgacgacatctcacgtgttgcccggacccctattcgcaagttgggttatgatgaacgtttcatccggccaattcgtgagctgaaggaacgtggcttaaattactcagttctgatggataccgttggtatgatgttccattatgttgaaccaaacgatgccgaagcagtaaagcttcaagccatgttgaaggatcaaccgttggtggacgttattaaggaagttacaggcttgaaggacgctggcctcattgatgaagtggaggcctcagttaaatcaaaggaccgttaa

SEQ ID NO: 41 - protein, 608 aa, Lactobacillus paracasei
MGAKTANTPAAEKKKFNLKAGMQSFGTKLSGMVLPNIGAFIAWGLITAIFLKGGWYPNAQLAKMISPMVTYLLPLLIAFSGGSMVAGHRGGVVGAIAAMGVIVGTDVPMFIGAMVMGPLGGWCIKKWDDRFQDKIKQGFEMLVNNFSAGIIGMLLAIVGFFLMGPIISTLTNGMATGVDWIINHGLLWVANVFIEPAKILFLNNAINQGILTPLGIQAAAEHGKSILFLLEPDPGPGLGVLLAFALFGKGSAKGSAPSAIIIHFLGGIHEIYFPYVLMKPALFLSVMAGGVTGTTLFSIFNVGLKSSPSPGSIFALFAMSPVNIGNYIGLIVGVTGATLVSFLISAVILRRDKSASGDELAESEAKMKSMKAEAKGQQNVAAAKDVMSAAKGIKQIIFACDAGMGSSAMGASILRDKVKKAGLDLSVTNTAISNLQDKPGLLVVTQEELADRAKDKTPDAAHIAVDNFLNSPKYDEIIASLKAEAVGGTDEAMPATETSKAKQETPEDELKELDLDKITEVDFLHHDQNIGSATMAQATFRAELRKLNKDVKVRNVAIGEIDDKDNVLIIASKETARRVKLQFANVQVYTVDGLLNATNYDKLIEKMK

SEQ ID NO: 42 - DNA, 1827 bp, synthetic construct
  misc_feature 1..1827: note = Encoding SEQ ID NO: 41 and codon-optimized to prevent homologous recombination with native host gene
  source 1..1827: mol_type = other DNA
atgggtgcaaagacggcaaatactccagcagcagaaaagaagaagttcaacctgaaggccggtatgcaaagcttcggtaccaagctttctggtatggttcttcctaacattggtgcctttatcgcttgggggttgatcacagctatctttctcaagggcggctggtatcctaatgcccagttggccaagatgatttctcctatggttacctacttgttaccgttgttgatcgcattctcgggtggttcaatggttgccggccatcgtggtggtgtcgtgggtgccattgctgccatgggtgttatcgttggtacagacgtcccaatgtttattggcgcaatggttatgggtcctttgggcggctggtgcatcaagaagtgggacgatcgcttccaggataagattaaacaaggcttcgaaatgctggttaacaacttcagcgcagggattattggcatgctgctggctatcgtcggctttttcctgatggggccaatcatttcaaccctgactaacggcatggctaccggtgttgattggattatcaaccatggcctcttgtgggttgctaacgtgttcattgaaccagctaagatcctattcctgaacaacgccatcaatcagggtatcttaacccctctgggcatccaggcagctgctgaacatggtaagagtatcttgtttctgctggaaccagaccctggccctgggcttggtgttttgttggccttcgctctgtttggcaagggtagcgccaaggggtccgccccgtcagccatcatcattcattttttgggcggcatccatgaaatctacttcccgtatgtcctgatgaagccagccttgtttttgtccgttatggctgggggtgtcaccggcactacattattttcaatcttcaatgttggtctaaaatcatcaccgagtcctggttcaatcttcgcactcttcgccatgagcccggtcaatattggcaattacatcggcttgatcgtcggtgtcaccggtgctacgttggtttctttcttgatctctgctgttatccttcgtcgcgataagtctgcatcgggtgatgaactggccgagtcagaagcaaagatgaagagcatgaaggcggaagctaagggtcaacagaatgttgcagcggccaaggatgttatgtccgcagcaaaaggtatcaagcaaattatcttcgcttgcgacgctggcatgggtagctctgcaatgggtgcatcaattttgcgtgataaggttaagaaggctgggctcgacctcagcgttaccaataccgcgatttcaaacttgcaggataaaccaggtcttttggttgtaacacaagaagaactggcggaccgtgcgaaggacaagaccccggatgcggctcatattgcagttgataacttcttgaatagccctaagtatgacgaaattattgcttctttgaaggctgaagccgtgggcggtacagatgaagctatgcctgcaaccgaaacctcaaaggcaaagcaagaaactcctgaagatgaattgaaagaattagatcttgacaaaatcacagaagttgactttcttcaccacgatcaaaatatcggttctgctactatggctcaagcaaccttccgtgccgaattgcggaagctcaataaagatgttaaggttcgtaatgtggctattggtgaaattgatgacaaggataatgtcttgattattgcatccaaggaaaccgcacgtcgtgtaaaattgcagttcgccaatgttcaagtttataccgttgatggccttttaaacgcgacaaactatgacaaattgatcgagaagatgaaataa

SEQ ID NO: 43 - protein, 158 aa, Lactobacillus paracasei
MKSKKLIEGDMMKGLDVKTIKLGQEAKTKEEAIRQAGQLLVDNGNVEPAYIDSMLDRNRDVSVYMGNFIAIPHGTEAGMKYIKSTAISIVQYPWGVDWSDDPADENLVTVVFGIAGLNGEHLKLLSQIALYCSDVENVQKLADAQTPEEIVNLLKEVE

SEQ ID NO: 44 - DNA, 477 bp, synthetic construct
  misc_feature 1..477: note = Encoding SEQ ID NO: 43 and codon-optimized to prevent homologous recombination with native host gene
  source 1..477: mol_type = other DNA
atgaaatctaagaagttgatcgaaggtgacatgatgaaaggattggatgttaaaacgatcaaacttggccaagaagccaaaacgaaggaagaagctatccgtcaggctggccaattgctcgttgataacggtaatgttgaaccagcttatattgattctatgttggaccgtaaccgcgacgttagtgtttacatgggcaatttcattgctattccacatgggacagaagctggtatgaagtacattaagagtacggctatttctatcgttcagtacccgtggggcgtggactggtcagacgacccggctgatgagaacttagttactgttgttttcggtattgccggtttgaacggtgaacacctgaagctgctgtctcagatcgcattgtattgctccgatgttgaaaacgttcaaaagttggcagatgctcagacgccagaagaaatcgtcaacttgttaaaggaagttgaataa

SEQ ID NO: 45 - protein, 360 aa, Entamoeba nuttalli
MKGLAMLGIGRIGWIEKKIPECGPLDALVRPLALAPCTSDTHTVWAGAIGDRHDMILGHEAVGQIVKVGSLVKRLKVGDKVIVPAITPDWGEEESQRGYPMHSGGMLGGWKFSNFKDGVFSEVFHVNEADANLALLPRDIKPEDAVMLSDMVTTGFHGAELANIKLGDTVCVIGIGPVGLMSVAGANHLGAGRIFAVGSRKHCCDIALEYGATDIINYKNGDIVEQILKATDGKGVDKVVIAGGDVHTFAQAVKMIKPGSDIGNVNYLGEGDNIDIPRSEWGVGMGHKHIHGGLTPGGRVRMEKLASLISTGKLDTSKLITHRFEGLEKVEDALMLMKNKPADLIKPVVRIHYDDEDTLH

SEQ ID NO: 46 - DNA, 1083 bp, synthetic construct
  misc_feature 1..1083: note = Encoding SEQ ID NO: 45 and codon-optimized for expression in Saccharomyces cerevisiae
  source 1..1083: mol_type = other DNA
atgaagggtttggctatgttgggtatcggtagaatcggttggatcgaaaagaagatcccagaatgtggtccattggacgctttggttagaccattggctttggctccatgtacttctgacactcacactgtttgggctggtgctatcggtgacagacacgacatgatcttgggtcacgaagctgttggtcaaatcgttaaggttggttctttggttaagagattgaaggttggtgacaaggttatcgttccagctatcactccagactggggtgaagaagaatctcaaagaggttacccaatgcactctggtggtatgttgggtggttggaagttctctaacttcaaggacggtgttttctctgaagttttccacgttaacgaagctgacgctaacttggctttgttgccaagagacatcaagccagaagacgctgttatgttgtctgacatggttactactggtttccacggtgctgaattggctaacatcaagttgggtgacactgtttgtgttatcggtatcggtccagttggtttgatgtctgttgctggtgctaaccacttgggtgctggtagaatcttcgctgttggttctagaaagcactgttgtgacatcgctttggaatacggtgctactgacatcatcaactacaagaacggtgacatcgttgaacaaatcttgaaggctactgacggtaagggtgttgacaaggttgttatcgctggtggtgacgttcacactttcgctcaagctgttaagatgatcaagccaggttctgacatcggtaacgttaactacttgggtgaaggtgacaacatcgacatcccaagatctgaatggggtgttggtatgggtcacaagcacatccacggtggtttgactccaggtggtagagttagaatggaaaagttggcttctttgatctctactggtaagttggacacttctaagttgatcactcacagattcgaaggtttggaaaaggttgaagacgctttgatgttgatgaagaacaagccagctgacttgatcaagccagttgttagaatccactacgacgacgaagacactttgcactaa

SEQ ID NO: 47 - protein, 910 aa, Bifidobacterium adolescentis; identical to SEQ ID NO: 15

SEQ ID NO: 48 - DNA, 2733 bp, synthetic construct
  misc_feature 1..2733: note = Encoding SEQ ID NO: 47 and codon-optimized for expression in Saccharomyces cerevisiae
  source 1..2733: mol_type = other DNA
atggccgacgccaagaagaaagaagaacctactaagccaaccccagaagaaaaattggctgctgctgaagctgaagttgatgctttggttaagaaaggtttgaaggccttggacgaattcgaaaaattggatcaaaagcaagtcgatcacatcgttgctaaagcttcagttgctgctttgaacaaacatttggttttggctaagatggccgttgaagaaactcatagaggtttggttgaagataaggccaccaagaatattttcgcttgtgaacatgtcaccaactatttggctggtcaaaagaccgttggtatcattagagaagatgatgttttgggtatcgacgaaattgctgaaccagttggtgttgttgctggtgttactccagttactaatccaacttctaccgctattttcaagtccttgattgccttgaaaaccagatgcccaattatctttggttttcatccaggtgctcaaaactgttctgttgctgctgctaaaatcgttagagatgctgctattgctgctggtgctccagaaaactgtattcaatggattgaacacccatccattgaagctactggtgctttgatgaagcacgatggtgttgctactattttggctactggtggtccaggtatggttaaggctgcttattcttctggtaaaccagctttgggtgttggtgctggtaatgctccagcttatgttgataagaacgttgatgttgttagagctgccaacgatttgattttgtctaagcacttcgactacggtatgatttgtgctactgaacaagctattatcgccgataaggatatctatgctccattggtcaaagaattgaagagaagaaaggcctacttcgttaatgctgacgaaaaagctaagttggaacagtatatgttcggttgtaccgcttactctggtcaaactccaaagttgaattctgttgttccaggtaagtccccacagtatattgctaaagctgccggtttcgaaattccagaagatgctacaattttggccgctgaatgtaaagaagtcggagaaaacgaaccattgaccatggaaaaattggcaccagttcaagctgttttgaagtccgataacaaagaacaagccttcgaaatgtgcgaagccatgttgaaacatggtgctggtcatactgctgctattcatacaaacgatagagacttggtcagagaatacggtcaaagaatgcatgcctgcagaattatttggaactctccatcttctttgggtggtgttggtgatatctacaatgctattgctccatctttgactttgggttgtggttcttatggtggtaattctgtttccggtaatgttcaagccgtcaacttgattaacatcaagagaatcgctagaagaaacaacaacatgcaatggttcaagattccagctaagacttactttgaacctaacgccatcaagtacctaagagatatgtacggtatcgaaaaggctgttatcgtttgcgataaggtcatggaacaattgggtatcgttgataagatcatcgatcaattgagagccagatctaacagagttaccttcagaatcatcgattacgttgaaccagaaccatctgttgaaacagttgaaaggggtgctgctatgatgagagaagaatttgaacctgataccattattgctgttggtggtggttctccaatggatgcttctaagattatgtggttgttgtacgaacacccagaaatttcattctccgatgtcagagaaaagttcttcgacattagaaagagagcctttaagattccaccattgggtaaaaaggccaagttggtatgtattccaacctcttcaggtactggttctgaagttactccattcgctgttattaccgatcataagactggttacaagtacccaattaccgattatgctttgactccatctgttgctatcgttgatccagttttggctagaactcaacctagaaaattggcttctgatgctggttttgatgctttgacacatgcttttgaagcctacgtttctgtttacgctaacgatttcactgatggtatggctttacatgctgctaaattggtttgggataacttggctgaatccgttaatggtgaaccaggtgaagaaaaaactagagcccaagaaaagatgcataacgctgctactatggctggtatggcatttggttctgcttttttgggtatgtgtcatggtatggctcatacaattggtgctttgtgtcatgttgctcatggtagaactaactccattttgttgccatacgtcatcagatacaacggttctgttcctgaagaacctacatcttggccaaagtacaacaagtatattgccccagaaagataccaagaaatcgctaagaacttgggtgttaatccaggtaaaactcctgaagaaggtgttgaaaatttggctaaggctgtcgaagattacagagataacaagttgggtatgaacaagtccttccaagaatgtggtgttgacgaagattactactggtccattatcgatcaaattggtatgagagcctacgaagatcaatgtgctccagctaatccaagaattccacaaatcgaagatatgaaggatattgctattgccgcttactacggtgtttctcaagctgaaggtcataagttgagagttcaaagacaaggtgaagctgctacagaagaagcttctgaaagagcttaa

SEQ ID NO: 49 - protein, 683 aa, Saccharomyces cerevisiae
MTIKEHKVVYEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 50 - DNA, 2052 bp, Saccharomyces cerevisiae
  source 1..2052: mol_type = other DNA
atgacaatcaaggaacataaagtagtttatgaagctcacaacgtaaaggctcttaaggctcctcaacatttttacaacagccaacccggcaagggttacgttactgatatgcaacattatcaagaaatgtatcaacaatctatcaatgagccagaaaaattctttgataagatggctaaggaatacttgcattgggatgctccatacaccaaagttcaatctggttcattgaacaatggtgatgttgcatggtttttgaacggtaaattgaatgcatcatacaattgtgttgacagacatgcctttgctaatcccgacaagccagctttgatctatgaagctgatgacgaatccgacaacaaaatcatcacatttggtgaattactcagaaaagtttcccaaatcgctggtgtcttaaaaagctggggcgttaagaaaggtgacacagtggctatctatttgccaatgattccagaagcggtcattgctatgttggctgtggctcgtattggtgctattcactctgttgtctttgctgggttctccgctggttcgttgaaagatcgtgtcgttgacgctaattctaaagtggtcatcacttgtgatgaaggtaaaagaggtggtaagaccatcaacactaaaaaaattgttgacgaaggtttgaacggagtcgatttggtttcccgtatcttggttttccaaagaactggtactgaaggtattccaatgaaggccggtagagattactggtggcatgaggaggccgctaagcagagaacttacctacctcctgtttcatgtgacgctgaagatcctctatttttattatacacttccggttccactggttctccaaagggtgtcgttcacactacaggtggttatttattaggtgccgctttaacaactagatacgtttttgatattcacccagaagatgttctcttcactgccggtgacgtcggctggatcacgggtcacacctatgctctatatggtccattaaccttgggtaccgcctcaataattttcgaatccactcctgcctacccagattatggtagatattggagaattatccaacgtcacaaggctacccatttctatgtggctccaactgctttaagattaatcaaacgtgtaggtgaagccgaaattgccaaatatgacacttcctcattacgtgtcttgggttccgtcggtgaaccaatctctccagacttatgggaatggtatcatgaaaaagtgggtaacaaaaactgtgtcatttgtgacactatgtggcaaacagagtctggttctcatttaattgctcctttggcaggtgctgtcccaacaaaacctggttctgctaccgtgccattctttggtattaacgcttgtatcattgaccctgttacaggtgtggaattagaaggtaatgatgtcgaaggtgtccttgccgttaaatcaccatggccatcaatggctagatctgtttggaaccaccacgaccgttacatggatacttacttgaaaccttatcctggtcactatttcacaggtgatggtgctggtagagatcatgatggttactactggatcaggggtagagttgacgacgttgtaaatgtttccggtcatagattatccacatcagaaattgaagcatctatctcaaatcacgaaaacgtctcggaagctgctgttgtcggtattccagatgaattgaccggtcaaaccgtcgttgcatatgtttccctaaaagatggttatctacaaaacaacgctactgaaggtgatgcagaacacatcacaccagataatttacgtagagaattgatcttacaagttaggggtgagattggtcctttcgcctcaccaaaaaccattattctagttagagatctaccaagaacaaggtcaggaaagattatgagaagagttctaagaaaggttgcttctaacgaagccgaacagctaggtgacctaactactttggccaacccagaagttgtacctgccatcatttctgctgtagagaaccaatttttctctcaaaaaaagaaataa

SEQ ID NO: 51 - protein, 571 aa, Millerozyma farinose
MGFELWGRTNTGGLRGRPLRVAITAVATTGFSLFGYDQGLMSGIITGTEFNEEFPPTWSKPHYNASEKRHATVVQGAVTACYEIGCFFGALFALVRGDRIGRRPLVIVGAVLIIIGTVISTAAFGEHWGLGQFVIGRVITGIGNGMNTATIPVWQSEISRPENRGKLVNLEGSVIAIGTFVAYWIDFGLSYVNSSVQWRFPVAFQIVFAAGLLGGILFMPESPRWLLAHGKKEQAHIVLGALNDLDPNDDHVLAESTVITDAINRFSRSQLGFKELMSGGKNQHFARMVIGSSTQFFQQFTGCNAAIYYSTVLFEETIFVGDRRLSLVMGGVFASVYALATIPSFFLVDKLGRRNLFLIGATGQALSFTITFACLINPTKQNAKGAAVGIFLFITFFAFTILPLPWIYPPEINPLRTRTVASAVSTCTNWLTNFAVVMFTPIFINDAQWGCYLFFACLNYAFIPVIFWFYPETAGRSLEEIDIIFAKAYTDGRPPWRVAATMPHLSLKEQEEQGMQLGLYDNEAEKQKFEQTENLMSSSSSAKLPEEGSNVNENENENTNEKDQTPKPTDV

SEQ ID NO: 52 - DNA, 1716 bp, synthetic construct
  misc_feature 1..1716: note = Encoding SEQ ID NO: 51 and codon-optimized for expression in Saccharomyces cerevisiae
  source 1..1716: mol_type = other DNA
atgggattcgaactttggggaaggaccaacacaggtggtttgagaggtagacctcttcgtgttgccatcaccgctgttgcaactactggtttctcccttttcggttatgatcagggtttgatgtctggtattattaccggtactgaatttaacgaggagttccctccaacctggtccaagccacattacaacgcgtctgagaagagacatgctactgttgttcaaggtgctgttacagcttgttacgaaattggttgtttcttcggtgctctttttgctttggttagaggtgacaggatcggtagacgtccacttgtcattgttggtgctgttcttatcatcattggtactgttatttctactgctgcttttggtgaacactggggtttgggtcaattcgttattggtagagttattactggtattggtaacggtatgaacacagcaactatcccagtctggcaatctgagatctctcgtccagaaaacagaggtaagttagtcaacttggaaggttcagtcattgccattggtactttcgttgcttactggattgatttcggtctctcctacgttaacagctctgtacaatggagattccctgttgcgttccaaattgtttttgctgctggacttcttggaggtattcttttcatgccggagtctcctagatggttgctcgctcatggcaagaaggagcaagcacacatagtcttaggtgctttgaatgatctcgaccctaatgatgaccatgtccttgctgagagtactgttattaccgatgctattaacagattctccaggtctcaacttggtttcaaggaacttatgtccggtggtaagaaccaacattttgctagaatggttattggttcttccactcaatttttccaacagttcactggttgtaatgctgccatttactattcaacagttttgttcgaagagaccattttcgtcggtgacagaagattgtctttggttatgggtggtgttttcgcttccgtatacgcccttgccactattccatctttcttcttagtcgataagcttggtagaagaaacttgttcttgattggtgctactggtcaagctttgtctttcaccattacatttgcttgtttgatcaacccaacaaagcaaaatgctaagggtgcagctgttggtatcttcttgtttatcaccttcttcgcctttacaattttgccattgccttggatttacccaccagaaatcaacccattgagaacaagaactgttgcctctgccgtttctacatgtaccaattggcttacaaactttgccgtcgttatgtttactcctattttcattaacgatgctcaatggggttgttacttgttctttgcttgtttgaactacgctttcattccagttatcttctggttctacccagaaactgctggccgttccttggaagaaattgatatcattttcgcgaaggcttacactgatggaagacctccatggagagttgctgctaccatgccacacttgtctttgaaggaacaagaggagcaaggtatgcaactcggactttatgacaatgaagctgagaaacagaagttcgagcaaaccgagaacttgatgtcttctagctcttctgcgaagcttcctgaagagggatctaacgtaaacgagaatgagaacgaaaacacgaacgaaaaggatcaaacaccaaagccaactgatgtttga

SEQ ID NO: 53 - protein, 569 aa, Saccharomyces cerevisiae; identical to SEQ ID NO: 11

SEQ ID NO: 54 - DNA, 1502 bp, Saccharomyces cerevisiae
  source 1..1502: mol_type = other DNA
atcatgacagacacgcaactgtagtgcagggcgctacaacctcctgttatgaattaggttgtttcgcaggttctctattcgttatgttctgcggtgaaagaattggtagaaaaccattaatcctgatgggttccgtaataaccatcattggtgccgttatttctacatgcgcatttcgtggttactgggcattaggccagtttatcatcggaagagtcgtcactggtgttggaacagggttgaatacatctactattcccgtttggcaatcagaaatgtcaaaagctgaaaatagagggttgctggtcaatttagaaggttccacaattgcttttggtactatgattgcttattggattgattttgggttgtcttataccaacagttctgttcagtggagattccccgtgtcaatgcaaatcgtttttgctctcttcctgcttgctttcatgattaaactacctgaatcgccacgttggctgatttctcaaagtcgaacagaagaagctcgctacttggtaggaacactagacgacgcggatccaaatgatgaggaagttataacagaagttgctatgcttcacgatgctgttaacaggaccaaacacgagaaacattcactgtcaagtttgttctccagaggcaggtcccaaaatcttcagagggctttgattgcagcttcaacgcaatttttccagcaatttactggttgtaacgctgccatatactactctactgtattattcaacaaaacaattaaattagactatagattatcaatgatcataggtggggtcttcgcaacaatctacgccttatctactattggttcattttttctaattgaaaagctaggtagacgtaagctgtttttattaggtgccacaggtcaagcagtttcattcacaattacatttgcatgcttggtcaaagaaaataaagaaaacgcaagaggtgctgccgtcggcttatttttgttcattacattctttggtttgtctttgctatcattaccatggatatacccaccagaaattgcatcaatgaaagttcgtgcatcaacaaacgctttctccacatgtactaattggttgtgtaactttgcggttgtcatgttcaccccaatatttattggacagtccggttggggttgctacttattttttgctgttatgaattatttatacattccagttatcttctttttctaccctgaaaccgccggaagaagtttggaggaaatcgacatcatctttgctaaagcatacgaggatggcactcaaccatggagagttgctaaccatttgcccaagttatccctacaagaagtcgaagatcatgccaatgcattgggctcttatgacgacgaaatggaaaaagaggactttggtgaagatagagtagaagacacctataaccaaattaacggcgataattcgtctagttcttcaaacatcaaaaatgaagatacagtgaacgataaagcaaattttgagggttga

SEQ ID NO: 55 - protein, 128 aa, Lactobacillus paracasei
MLRKFKITIDGKTYLVEMEEIGGAPAAQPAPAAPAATPTPAPAAPAAPAPAAPVAPTGEGEVVTAPMPGTVTKILVKDGDAVTENQPLMILEAMKMENEIVAPKAGTIGQVFATLNQNVNSGDNLISI

SEQ ID NO: 56 - DNA, 390 bp, Lactobacillus paracasei
  source 1..390: mol_type = other DNA
atgttgagaaaattcaagatcacgattgatgggaaaacctatttggtcgaaatggaagaaattggcggtgcgccagccgcccagcctgcgccggccgcaccagcggcaacgccgacaccggcaccggccgcaccagctgcgccagcacctgcagctccggttgcgccgactggggaaggtgaagttgtcactgcaccaatgccaggcacggtcaccaagattttggttaaagacggtgatgcagtcacggaaaatcagccgctgatgattctggaagccatgaagatggaaaacgaaattgtggcgcctaaggcaggtaccatcggccaggtgtttgcaacacttaaccagaatgtcaattccggcgacaatctcatcagcattatttaa

(19) United States
(12) Patent Application Publication
(10) Pub. No.: US 2025/0263756 A1
(43) Pub. Date: Aug. 21, 2025

(54) SYNERGISTIC BACTERIAL AND YEAST COMBINATIONS

(71) Applicant: Lallemand Hungary Liquidity Management LLC, Budapest (HU)

(72) Inventors: Jeffery R. Broadbent, Amalga, UT (US); Aaron Argyros, Lebanon, NH (US); Brooks Henningsen, Salisbury, NH (US); Fernanda Cristina Firmino, Atlanta, GA (US); Ekkarat Phrommao, Lebanon, NH (US); James L. Steele, Lebanon, NH (US)

(21) Appl. No.: 18/967,018

(22) Filed: Dec. 3, 2024

Related U.S. Application Data
(63) Continuation of application No. 17/292,358, filed on May 7, 2021, now abandoned.
(60) Provisional application No. 62/760,472, filed on Nov. 13, 2018.

(51) Int. Cl. C12P 7/14; C12N 9/88; C12P 7/10; C12P 7/54; C12P 19/02

(52) U.S. Cl. CPC C12P 7/14; C12N 9/88; C12P 7/10; C12P 7/54; C12P 19/02

(57) ABSTRACT

The present disclosure concerns a symbiotic combination of host cells engineered to produce a first metabolic product, for example a carbohydrate, and to convert the second metabolic product into a second metabolic product, for example an alcohol.

### CROSS-REFERENCE TO RELATED APPLICATIONS

The present application is a continuation of U.S. patent application Ser. No. 17/292,358, filed on May 7, 2021, which is a U.S. national phase application of PCT/IB2019/059765 filed on Nov. 13, 2019, which claims priority from U.S. provisional patent application 62/760,472 filed on Nov. 13, 2018 all of which are herewith incorporated by reference in their entirety.

### REFERENCE TO AN ELECTRONIC SEQUENCE LISTING

The contents of the electronic sequence (580127_423C1_SEQUENCE_LISTING.xml.; Size 101,601 bytes; and Date of Creation: Dec. 3, 2024) is herein incorporated by reference in its entirety.

### TECHNOLOGICAL FIELD

The present disclosure concerns a combination of a bacterial host cell and a yeast host cell exhibiting a symbiotic relationship to convert a first metabolic product into a second metabolic product.

### BACKGROUND

Interactions between various microorganisms have been well characterized in numerous diverse environments, ranging from food and beverage production to clinical settings. These interactions can be either antagonistic or symbiotic in nature and play a significant role in the balance of microbial ecosystems. Symbiotic interactions may be mutualistic, wherein both organisms benefit, or commensal, where only one benefits. One example of a symbiotic relationship includes the production and secretion of metabolites by one organism that are utilized by another (Schink, 2002). The subsequent organism benefits either due to their lack of the enzymes required for the synthesis of the metabolite or through conservation of energy that would otherwise be required to synthesize it de novo.

These microbial interactions occur both within and across phylogenetic kingdoms and several reports of yeast-bacterial interactions have been documented (Peleg et al., 2010; Wargo and Hogan, 2006). The yeast, Saccharomyces cerevisiae, is utilized as the primary bio-catalyst in commercial bioethanol production, however, diverse populations of lactic acid bacteria (LAB) are also ubiquitous within the fermentation vessels. The impacts of LAB on yeast fermentation have typically been shown to be antagonistic leading to decreased ethanol titers and stuck fermentations. Antibiotics are therefore heavily utilized within the industry to try and mitigate infections. However, the use of antibiotics raises concerns related to the selection of resistant bacterial strains and the presence of antibiotics in fermentation residuals that are sold as animal feed.

For instance, Lactobacillus paracasei strain 12A robustly utilizes trehalose even when glucose is readily available. Trehalose is a common constituent of residual DP2 sugars (sugars with degree of polymerization=2) in corn fermentations. Saccharomyces cerevisiae often synthesizes trehalose in response to stress and previous studies have indicated that up-regulation of trehalose biosynthesis improves yeast robustness. Unfortunately, trehalose accumulation by the yeast is known to subtract from ethanol yield as glucose-6-phosphate is diverted from central metabolism through the enzymes TPS1 and TPS2 (Yi et al., 2016).

It would be highly desirable to be provided with means of increasing alcohol production during yeast fermentation that would exploit, rather than limit, the symbiotic relationship between yeasts and bacteria, especially lactic acid bacteria.

### BRIEF SUMMARY

The present disclosure concerns a symbiotic combination of a yeast host cell and a bacterial host cell. The symbiotic combination cell has the ability or is engineered to make a first metabolic product intended to be used by the second microbial host cell to make a second metabolic product. In some embodiments, the symbiotic combination achieve higher fermentation yield (when compared for example from a fermentation conducted in the absence of the bacterial cell). In some embodiments, the symbiotic combination of the present disclosure provides higher robustness.

According to a first aspect, the present disclosure provides a combination of a first microbial host cell having a first metabolic pathway comprising one or more first enzymes for producing a first metabolic product and a second microbial host cell having a second metabolic pathway comprising one or more second enzymes for converting at least in part the first metabolic product into a second metabolic product. In such combination, at least one of the first microbial host cell or the second microbial host cell is recombinant; at least one of the first microbial host cell or the second microbial host cell is a bacterial host cell; and at least one of the first microbial host cell or the second microbial host cell is a yeast host cell. In the combinations of the present disclosure, when the first microbial host cell is a recombinant first microbial host cell, the recombinant first microbial host cell has increased activity in the first metabolic pathway, when compared to a corresponding native first microbial host cell, for producing the first metabolic product. Still in the combinations of the present disclosure, when the second microbial host cell is a recombinant second microbial host cell, the recombinant second microbial host cell has increased activity in the second metabolic pathway, when compared to a corresponding native second microbial host cell, for converting at least in part the first metabolic product into the second metabolic product. In an embodiment, the first microbial host cell is a bacterial host cell and the second microbial cell is a yeast host cell. As such, the present disclosure provides a combination of a bacterial host cell having a first metabolic pathway comprising one or more first enzymes for producing a first metabolic product and a yeast host cell having a second metabolic pathway comprising one or more second enzymes for converting at least in part the first metabolic product into a second metabolic product, wherein at least one of the bacterial host cell or the yeast host cell is recombinant. When the bacterial host cell is a recombinant bacterial host cell, the recombinant bacterial host cell has increased activity in the first metabolic pathway, when compared to a corresponding native bacterial host cell, for producing the first metabolic product. When the yeast host cell is a recombinant yeast host cell, the recombinant yeast host cell has increased activity in the second metabolic pathway, when compared to a corresponding native yeast host cell, for converting at least in part the first metabolic product into the second metabolic product. In an embodiment, at least one of the one or more first enzymes are native enzymes. In another embodiment, at least one of the one or more second enzymes are heterologous enzymes. In an embodiment, the first metabolic product is an organic ester, such as, for example, acetate. In another embodiment, the second metabolic product is ethanol. In an embodiment, the one or more first enzymes comprises a citrate lyase.

In some embodiments, the yeast host cell is the recombinant yeast host cell and the one or more second enzyme comprises a polypeptide having an heterologous polypeptide having acetylating acetaldehyde dehydrogenase activity. The polypeptide having acetylating acetaldehyde dehydrogenase activity is an acetylating acetaldehyde dehydrogenase (AADH) or a bifunctional acetylating ace/alcohol dehydrogenase (ADHE). In specific embodiments, the polypeptide having acetylating aldehyde dehydrogenase activity is heterologous bifunctional acetaldehyde/alcohol dehydrogenase (ADHE) having, in some embodiments, the amino acid sequence of SEQ ID NO: 15, being a variant of the amino acid sequence of SEQ ID NO: 15 having acetaldehyde/alcohol dehydrogenase activity or being a fragment of the amino acid sequence of SEQ ID NO: 15 having acetaldehyde/alcohol dehydrogenase activity. In some embodiments, the one or more second enzymes comprises an heterologous polypeptide having NADP⁺-dependent alcohol dehydrogenase activity (e.g., NADPH-ADH which can be, for example, ADH1 which can be obtained from Entamoeba sp., including Entamoeba nuttalli) or a polypeptide encoded by an adhl gene ortholog). In an embodiment, heterologous polypeptide having NADP⁺-dependent alcohol dehydrogenase activity has the amino acid sequence of SEQ ID NO: 45, is a variant of the amino acid sequence of SEQ ID NO: 45 exhibiting NADP⁺-dependent alcohol dehydrogenase activity or is a fragment of the amino acid sequence of SEQ ID NO: 45 exhibiting NADP⁺-dependent alcohol dehydrogenase activity. In some embodiments, the one or more second enzymes comprise an heterologous polypeptide having acetyl-coA synthetase activity (which can be, for example ACS2 or a polypeptide encoded by an acs2 gene ortholog). In an embodiment, the heterologous polypeptide having acetyl-coA synthetase activity has the amino acid sequence of SEQ ID NO: 49, is a variant of the amino acid sequence of SEQ ID NO: 49 exhibiting acetyl-coA synthetase activity or is a fragment of the amino acid sequence of SEQ ID NO: 49 exhibiting acetyl-coA synthetase activity.

In some embodiments, the first microbial host cell is a yeast host cell and the second microbial host cell is a bacterial host cell. As such, the present disclosure provides a combination of a yeast host cell having a first metabolic pathway comprising one or more first enzymes for producing a first metabolic product and a bacterial host cell having a second metabolic pathway comprising one or more second enzymes for converting at least in part the first metabolic product into a second metabolic product, wherein at least one of the yeast host cell or the bacterial host cell is recombinant. When the yeast host cell is a recombinant yeast host cell, the recombinant yeast host cell has increased activity in the first metabolic pathway, when compared to a corresponding native yeast host cell, for producing the first metabolic product. When the bacterial host cell is a recombinant bacterial host cell, the recombinant bacterial host cell has increased activity in the second metabolic pathway, when compared to a corresponding native bacterial host cell, for converting at least in part the first metabolic product into the second metabolic product. In an embodiment, at least one of the one or more first enzymes are heterologous enzymes. In another embodiment, at least one of the one or more second enzymes are heterologous enzymes. In an embodiment, the first metabolic product is a carbohydrate. In another embodiment, the second metabolic product is ethanol.

In a specific embodiment, the carbohydrate is trehalose. In such embodiment, the one or more first enzymes comprises a trehalose-6-phosphate synthase, such as, for example, TPS1. In such embodiment, the one or more first enzymes comprises a trehalose-6-phosphate phosphatase, such as, for example, TPS2. In such embodiment, the one or more second enzymes comprises a pyruvate decarboxylase. The pyruvate decarboxylase can have, in some embodiments, the amino acid sequence of SEQ ID NO: 4, be a variant of the amino acid sequence of SEQ ID NO: 4 having pyruvate decarboxylase activity or be a fragment of the amino acid sequence of SEQ ID NO: 4 having pyruvate decarboxylase activity. In such embodiments, the one or more second enzymes comprises an alcohol dehydrogenase. The alcohol dehydrogenase can have, in some embodiments, the amino acid sequence of SEQ ID NO: 8, be a variant of the amino acid sequence of SEQ ID NO: 8 having alcohol dehydrogenase activity or be a fragment of the amino acid sequence of SEQ ID NO: 8 having alcohol dehydrogenase activity. In an embodiment, the bacterial host cell has a decreased lactate dehydrogenase activity when compared to the corresponding native bacterial host cell. In a further embodiment, the bacterial host cell has at least one inactivated native gene coding for a lactate dehydrogenase, such as, for example ldh1, ldh2, ldh3 or ldh4. In yet another embodiment, the bacterial host cell has a decreased mannitol dehydrogenase activity. In some embodiments, the bacterial host cell has at least one inactivated native gene coding for a mannitol-1-phosphate 5-dehydrogenase, such as, for example, mltD1 or mltD2.

In another specific embodiment, the carbohydrate is mannitol. In such embodiment, the one or more first enzymes comprises a mannitol-1-phosphate 5-dehydrogenase. In such embodiment, the one or more first enzymes comprises a MTLD enzyme. In some embodiments, the MTLD polypeptide can have the amino acid sequence of SEQ ID NO: 27, be a variant of the amino acid sequence of SEQ ID NO: 27 or be a fragment of the amino acid sequence of SEQ ID NO: 27 or a variant thereof. In some additional embodiments, the MTLD polypeptide can be encoded by an heterologous nucleic acid molecule having the nucleic acid sequence of SEQ ID NO: 28, a variant of the nucleic acid sequence of SEQ ID NO: 28 or a fragment of the nucleic acid sequence of SEQ ID NO: 28 or a fragment thereof. In such embodiment, the one or more second enzymes comprise at least one gene from a mannitol utilization operon. In yet another embodiment, the one or more second enzymes comprise mannitol-1-phophatase 5-dehydrogenase. In still another embodiment, the one or more second enzymes comprise a MTLD2 polypeptide. In an embodiment, the MTLD2 polypeptide can be from Lactobacillus sp., such as, for example Lactobacillus casei. In some embodiments, the MTLD2 polypeptide can have the amino acid sequence of SEQ ID NO: 39, be a variant of the amino acid sequence of SEQ ID NO: 39 or be a fragment of the amino acid sequence of SEQ ID NO: 39 or a variant thereof. In some additional embodiments, the MTLD2 polypeptide can be encoded by an heterologous nucleic acid molecule having the nucleic acid sequence of SEQ ID NO: 40, a variant of the nucleic acid sequence of SEQ ID NO: 40 or a fragment of the nucleic acid sequence of SEQ ID NO: 40 or a fragment thereof.

In another embodiment, the one or more second enzymes comprises a mannitol transporter. In some embodiments, the mannitol transporter comprises at least one of the MTLCB polypeptide or the MTLA polypeptide. In an embodiment, the MTLCB polypeptide can be from Lactobacillus sp., such as, for example Lactobacillus casei. In some embodiments, the MTLCB polypeptide can have the amino acid sequence of SEQ ID NO: 41, be a variant of the amino acid sequence of SEQ ID NO: 41 or be a fragment of the amino acid sequence of SEQ ID NO: 41 or a variant thereof. In some additional embodiments, the MTLCB polypeptide can be encoded by an heterologous nucleic acid molecule having the nucleic acid sequence of SEQ ID NO: 42, a variant of the nucleic acid sequence of SEQ ID NO: 42 or a fragment of the nucleic acid sequence of SEQ ID NO: 42 or a fragment thereof. In an embodiment, the MTLA polypeptide can be from Lactobacillus sp., such as, for example Lactobacillus casei. In some embodiments, the MTLA polypeptide can have the amino acid sequence of SEQ ID NO: 43, be a variant of the amino acid sequence of SEQ ID NO: 43 or be a fragment of the amino acid sequence of SEQ ID NO: 43 or a variant thereof. In some additional embodiments, the MTLA polypeptide can be encoded by an heterologous nucleic acid molecule having the nucleic acid sequence of SEQ ID NO: 44, a variant of the nucleic acid sequence of SEQ ID NO: 44 or a fragment of the nucleic acid sequence of SEQ ID NO: 44 or a fragment thereof.

In another specific embodiment, the carbohydrate is sorbitol. In such embodiment, the one or more first enzymes comprises a sorbitol-6-phosphate dehydrogenase (SRLD). In an embodiment, the one or more first enzymes comprises a SRLD enzyme. In still another embodiment, the one or more second enzymes comprises at least one gene from a sorbitol utilization operon, such as, for example, at least one of a gutF, a gutC, a gutB and/or a gutA gene. In an embodiment, the GUTF polypeptide is from Lactobacillus sp., such as, for example Lactobacillus paracasei. In such embodiment, the GUTF polypeptide can have, for example, the amino acid sequence of SEQ ID NO: 31, be a variant of the amino acid sequence of SEQ ID NO: 31 or be a fragment of the amino acid sequence of SEQ ID NO: 31 or a variant thereof. In an embodiment, the GUTF polypeptide is encoded by an heterologous nucleic acid molecule having the nucleic acid sequence of SEQ ID NO: 32, being a variant of the nucleic acid sequence of SEQ ID NO: 32 or being a fragment of the nucleic acid sequence or SEQ ID NO: 32 or a variant thereof. In an embodiment, the GUTC polypeptide is from Lactobacillus sp., such as, for example Lactobacillus paracasei. In such embodiment, the GUTC polypeptide can have, for example, the amino acid sequence of SEQ ID NO: 33, be a variant of the amino acid sequence of SEQ ID NO: 33 or be a fragment of the amino acid sequence of SEQ ID NO: 33 or a variant thereof. In an embodiment, the GUTC polypeptide is encoded by an heterologous nucleic acid molecule having the nucleic acid sequence of SEQ ID NO: 34, being a variant of the nucleic acid sequence of SEQ ID NO: 34 or being a fragment of the nucleic acid sequence or SEQ ID NO: 34 or a variant thereof. In an embodiment, the GUTB polypeptide is from Lactobacillus sp., such as, for example Lactobacillus paracasei. In such embodiment, the GUTB polypeptide can have, for example, the amino acid sequence of SEQ ID NO: 35, be a variant of the amino acid sequence of SEQ ID NO: 35 or be a fragment of the amino acid sequence of SEQ ID NO: 35 or a variant thereof. In an embodiment, the GUTB polypeptide is encoded by an heterologous nucleic acid molecule having the nucleic acid sequence of SEQ ID NO: 36, being a variant of the nucleic acid sequence of SEQ ID NO: 36 or being a fragment of the nucleic acid sequence or SEQ ID NO: 36 or a variant thereof. In an embodiment, the GUTA polypeptide is from Lactobacillus sp., such as, for example Lactobacillus paracasei. In such embodiment, the GUTA polypeptide can have, for example, the amino acid sequence of SEQ ID NO: 37, be a variant of the amino acid sequence of SEQ ID NO: 37 or be a fragment of the amino acid sequence of SEQ ID NO: 37 or a variant thereof. In an embodiment, the GUTA polypeptide is encoded by an heterologous nucleic acid molecule having the nucleic acid sequence of SEQ ID NO: 38, being a variant of the nucleic acid sequence of SEQ ID NO: 38 or being a fragment of the nucleic acid sequence or SEQ ID NO: 38 or a variant thereof.

In another specific embodiment, the carbohydrate is glycerol. In an embodiment, the second metabolic pathway comprises a glycerol dehydrogenase/DHA kinase pathway. In such embodiment, the one or more second enzymes comprise at least one of a glycerol dehydrogenase or a dihydroxyacetone kinase. In another embodiment, the second metabolic pathway comprises a glycerol kinase/glycerol-3-phosphate dehydrogenase pathway. In such embodiment, the one or more second enzymes comprise at least one of a glycerol kinase or a glycerol-3-phosphate dehydrogenase. In such embodiment, the one or more second enzymes comprises a glycerol facilitator. In an embodiment, the yeast host cell has increased activity, when compared to the corresponding native yeast host cell, in an NADP⁺-dependent aldehyde dehydrogenase, such as, for example ALD6. In embodiment, the yeast host cell has increased activity, when compared to the corresponding native yeast host cell, in a phosphoketolase.

In the combinations of the present disclosure, the yeast host cell can be from Saccharomyces sp., such as, for example, Saccharomyces cerevisiae. In an embodiment, the bacterial host cell is a lactic acid bacterium.

In some embodiments, the bacterial host cell further comprises a third metabolic pathway comprising one or more third enzymes for producing a third metabolic product. In such embodiment, the bacterial host cell is the recombinant bacterial host cell and has increased activity in the third metabolic pathway, when compared to the corresponding native bacterial host cell, for producing the third metabolic product. In some embodiments, the third metabolic product is ethanol. In some additional embodiments, the one or more third enzymes for producing the third metabolic product comprises a pyruvate decarboxylase. In some embodiments, the pyruvate decarboxylase has the amino acid sequence of SEQ ID NO: 4, is a variant of the amino acid sequence of SEQ ID NO: 4 having pyruvate decarboxylase activity or is a fragment of the amino acid sequence of SEQ ID NO: 4 having pyruvate decarboxylase activity. In yet another embodiment, the one or more third enzymes comprises an alcohol dehydrogenase. In some embodiments, the alcohol dehydrogenase has the amino acid sequence of SEQ ID NO: 8, is a variant of the amino acid sequence of SEQ ID NO: 8 having alcohol dehydrogenase activity or is a fragment of the amino acid sequence of SEQ ID NO: 8 having alcohol dehydrogenase activity. In yet another embodiment, the bacterial host cell has a decreased lactate dehydrogenase activity when compared to the corresponding native bacterial host cell. In specific embodiments, the bacterial host cell has at least one inactivated native gene coding for a lactate dehydrogenase, such as, for example, ldh1, ldh2, ldh3 or ldh4. In some embodiments, the bacterial host cell has decreased mannitol dehydrogenase activity. In specific embodiments, the bacterial host cell has at least one inactivated native gene coding for a mannitol-1-phosphate 5-dehydrogenase, such as, for example, mltD1 or mltD2.

The bacterial host cell can be from Lactobacillus sp., such as, for example, Lactobacillus paracasei. The yeast host cell and/or the bacterial host cell can be provided as a cell concentrate. For example, the yeast host cell can be provided as a cream. In another example, the bacterial host cell can be provided as a frozen cell concentrate.

According to a third aspect, the present disclosure provides a process for converting a biomass into a fermentation product, the process comprises contacting the biomass with the combination defined herein under condition to allow the conversion of at least a part of the biomass into the fermentation product. In an embodiment, the biomass comprises corn, such as, for example, a corn provided as a mash. In another embodiment, the biomass comprises or is supplemented with citric acid and/or citrate. In an embodiment, the fermentation product is ethanol. In yet another embodiment, the process is conducted, at least in part, at a temperature higher than 31° C.

According to a fourth aspect, the present disclosure provides a commercial package comprising (i) the combination defined herein and (ii) instructions to perform the process defined herein. In an embodiment, the commercial package further comprises a fermentation medium comprising a biomass, such as, for example, a biomass comprising corn. In another embodiment, the commercial package further comprises citric acid and/or citrate.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates an embodiment of a metabolic engineering strategy for trehalose production by yeast host cell and subsequent metabolism by a bacterial host cell. Pathway components in black solid lines represent metabolic reactions that occur in the yeast host cell and the bacterial host cell to produce ethanol from glucose. The pathway identified by dotted lines (from glucose-6-P to trehalose) is used to promote trehalose production by the yeast host cell, and the pathway identified by dashed lines (from trehalose to glucose-6-P) shows how trehalose is metabolized by the bacterial host cell.

FIG. 2 illustrates an embodiment of a metabolic engineering strategy for utilization of yeast-derived glycerol by a bacterial host cell. The pathway identified black solid lines font represent metabolic reactions that occur in the yeast host cell and the bacterial host cell to produce ethanol from glucose. The pathway identified by dotted lines (from dihydroxyacetone-P to glycerol) is used by yeast host cell for glycerol production, and the pathway identified in dashed lines (from glycerol to dihydroxyacetone-P) shows strategies used to metabolically engineer the bacterial host cell to metabolize glycerol.

FIG. 3 illustrates an embodiment of a metabolic engineering strategy for mannitol production by a yeast host cell and subsequent metabolism by a bacterial host cell. The pathway components in solid font represent metabolic reactions that occur in the yeast and the bacterial host cell to produce ethanol from glucose. The pathway identified in dotted lines (from fructose-6-P to mannitol) is used to promote mannitol production by the yeast host cell, and the pathway identified by the dashed lines (from mannitol to fructose-6-P) shows how mannitol can be metabolized by the bacterial host cell.

FIG. 4 illustrates an embodiment of a metabolic engineering strategy for sorbitol production by yeast host cell and subsequent metabolism by a bacterial host cell. The pathway components in black solid font represent metabolic reactions that occur in the yeast and the bacterial host cells to produce ethanol from glucose. The pathway identified in dotted lines (from fructose to sorbitol) is used to promote sorbitol production by the yeast host cell, and the pathway identified by dashed lines (from sorbitol to fructose) shows how sorbitol is metabolized by the bacterial host cell.

FIG. 5 illustrates that improved yeast robustness can be achieved from both trehalose overexpression and co-fermentation with ethanologen strain E3.1. Ethanol (left Y axis in g/L, bars) and glucose (right Y axis in g/L, ♦) concentrations following 50 hours of fermentation in commercial corn mash are shown in both standard (permissive) and high temperature conditions. Strain M12156 was not modified to produce additional amounts of trehalose, while strain M16807 was modified to produce additional amounts of trehalose (by expressing TPS1 and TPS2) (refer to Table 1 for a description of the strains used).

FIG. 6 illustrates improved fermentation yield can be achieved from both sorbitol overexpression and co-fermentation with ethanologen strain M19605. Ethanol (left Y axis in g/L, bars), glucose (right Y axis in mM, ●), glycerol (right axis in mM, ▪) and sorbitol (right axis in mM, ▴) concentrations following 67 hours of fermentation in a modified chemically defined medium are shown. Results are shown with respect to the strains or combination of strains tested. Strain M2390 is a wild-type strain, while strain M20043 has been modified to express SRLD (see Table 4 for a description of the strains used).

FIG. 7 illustrates improved fermentation yield can be achieved from both mannitol overexpression and co-fermentation with ethanologen strain M19998. Ethanol (left Y axis in g/L, bars), glucose (right Y axis in mM, ●), glycerol (right axis in mM, ▪) and mannitol (right axis in mM, ▴) concentrations following 67 hours of fermentation in a modified chemically defined medium are shown. Results are shown with respect to the strains or combination of strains tested. Strain M2390 is a wild-type strain, while strain M20036 has been modified to express MTLD (see Table 4 for a description of the strains used).

FIG. 8 illustrates an embodiment of a metabolic engineering strategy for utilization of bacterial-derived citrate by a yeast host cell. The pathway identified black solid lines font represent metabolic reactions that occur in the yeast and the bacterial host cell. The pathway identified by dotted lines (from acetate to acetaldehyde) is used by yeast host cell for ethanol production, and the pathway identified in dashed lines (from citrate to acetate) shows strategies used to metabolically engineer the bacterial host cell to metabolize citrate.

FIG. 9 illustrates the metabolite profiles of Lb. paracasei 12A and derived ethanologen E5 in after fermentation for 68 h in mCDM medium supplemented with 50 mM glucose (pH 6.5). Results are shown as the net mM of glucose, lactic acid, acetic acid, ethanol and citric acid in function of the strain tested.

FIG. 10 illustrates the metabolite profiles of S. cerevisiae strains M8279 and M10909 (alone or in combination with Lb. paracasei strain M20896) after fermentation for 68 h in mCDM medium supplemented with 50 mM glucose (pH 6.5). Results are shown as the net mM of ethanol (left axis), glycerol acetic acid, residual glucose and residual citrate in function of the strain tested.

FIG. 11 illustrates the percent increase in ethanol yield (ethanol/glucose) and percent glycerol reduction of S. cerevisiae strains M8279 and M10909 (alone or in combination with Lb. paracasei strain M20896) after fermentation for 68 h in mCDM medium supplemented with 50 mM glucose (pH 6.5) without and with the presence of citrate. Results are shown as the percent increase in ethanol yield (ethanol/glucose, left axis) and percent glycerol reduction in function of the strain tested and the presence or absence of citrate.

### DETAILED DESCRIPTION

The present disclosure concerns a combination of a yeast host cell and a bacterial host cell wherein one of the host cell is a recombinant host cell. One of the host cell has a first metabolic pathway comprising one or more first enzymes for producing a first metabolic product. The other host cell has a second metabolic pathway comprising one or more second enzymes for converting (at least in part) the first metabolic product into a second metabolic product. In an embodiment, the combination provides increased robustness to the yeast host cell in response to a stressor, such as for example elevated temperatures.

In some embodiments of the combinations of the present disclosure, the yeast host cell has the ability or is engineered to make a first metabolite product intended to be utilized by the bacterial host cell (to make the second metabolic product). When the yeast host cell is recombinant (e.g., engineered to make the first metabolite product), it has an increased activity in the first metabolic pathway when compared to the native or parental yeast host cell (which has been used to engineer the recombinant yeast host cell and which lacks the genetic modification(s) associated to increase the activity in the first metabolic pathway). In such embodiment, the bacterial host cell has the ability or is engineered to make a second metabolite from the first metabolite produced at least in part by the yeast host cell. When the bacterial host cell is recombinant (e.g., engineered to make the second metabolite product), it has an increased activity in the second metabolic pathway when compared to the native or parental bacterial host cell (which has been used to engineer the recombinant bacterial host cell and which lacks the genetic modification(s) associated to increase the activity in the second metabolic pathway). In specific embodiments, the first metabolic product is made from a molecule that is used to produce a fermentation product (an alcohol such as ethanol).

In alternative embodiments of the combinations of the present disclosure, the bacterial host cell has the ability or is engineered to make a first metabolite product intended to be utilized by the yeast host cell (to make the second metabolic product). When the bacterial host cell is recombinant (e.g., engineered to make the first metabolite product), it has an increased activity in the first metabolic pathway when compared to the native or parental bacterial host cell (which has been used to engineer the recombinant bacterial host cell and which lacks the genetic modification(s) associated to increase the activity in the first metabolic pathway). In such embodiment, the yeast host cell has the ability or is engineered to make a second metabolite from the first metabolite produced at least in part by the bacterial host cell. When the yeast host cell is recombinant (e.g., engineered to make the second metabolite product), it has an increased activity in the second metabolic pathway when compared to the native or parental yeast host cell (which has been used to engineer the recombinant yeast host cell and which lacks the genetic modification(s) associated to increase the activity in the second metabolic pathway). In specific embodiments, the first metabolic product is made from a molecule that is used to produce a fermentation product (an alcohol such as ethanol).

In specific embodiments, the second metabolic product can be used in the production of a fermentation product (an alcohol such as ethanol). In some embodiments, the combinations of the present disclosure are useful for recycling a yeast osmo-protectant (trehalose, mannitol, sorbitol and/or glycerol for example) into a fermentation product (such as ethanol). In some embodiments, the yeast/bacterial relationship promotes the production of a fermentation product, such as, for example, an alcohol (e.g., ethanol).

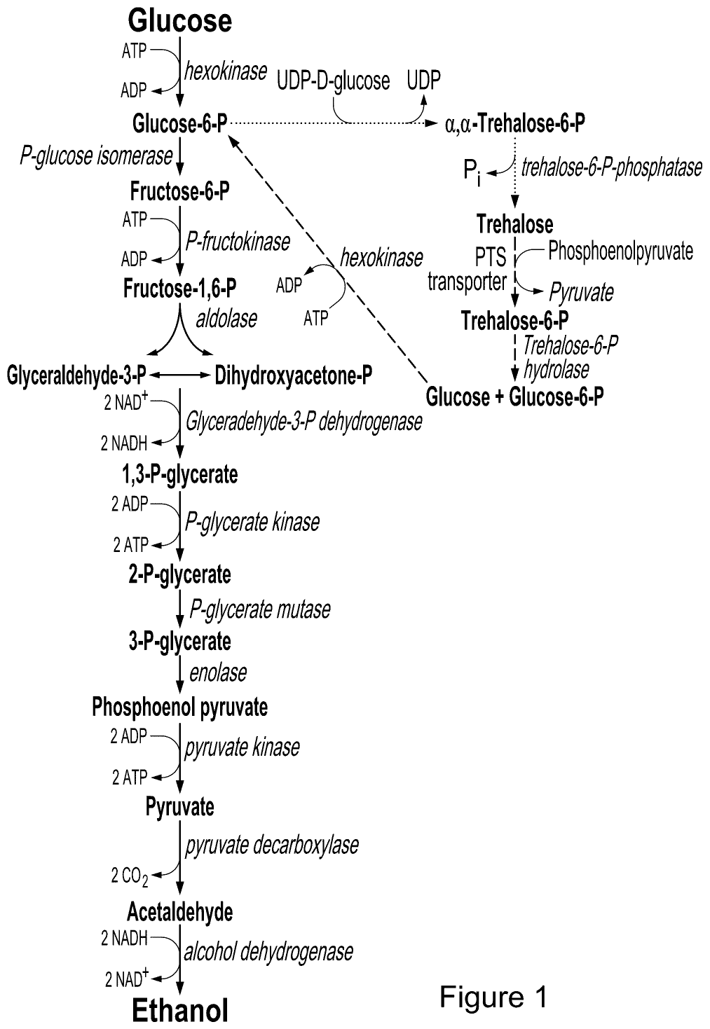

In one embodiment, shown on FIG. 1, the first metabolic product produced by the yeast host cell can be trehalose which can subsequently be metabolized to ethanol (e.g., the second metabolic product) by the bacterial host cell. When the second metabolic product is ethanol, the yeast host cell can be selected based on its ability to convert glucose-6-phosphate into α,α-trehalose-6-phosphate (α,α-trehalose-6-P), α,α-trehalose-6-P into trehalose (via the activity of one or more a trehalose-6-phosphatase). In some embodiments, the yeast host cell can be genetically modified to provide or increase its ability to convert glucose-6-phosphate into α,α-trehalose-6-phosphate (α,α-trehalose-6-P) and/or α,α-trehalose-6-P into trehalose (via the activity of one or more a trehalose-6-phosphatase). In the embodiment shown on FIG. 1, when the second metabolic product is ethanol, the bacterial host cell can be selected based on its ability to convert trehalose into trehalose-6-phosphate (trehalose-6-P, via the activity or one or more PTS transporter), trehalose-6-P into glucose and glucose-6-P (via the activity of one or more trehalose-6-phosphate hydrolase) and glucose into glucose-6-P (via the activity of one or more hexokinase). In some embodiments, the bacterial host cell is genetically modified to provide or increase its ability to convert trehalose into trehalose-6-phosphate (trehalose-6-P, via the activity or one or more PTS transporter), trehalose-6-P into glucose and glucose-6-P (via the activity of one or more trehalose-6-phosphate hydrolase) and/or glucose into glucose-6-P (via the activity of one or more hexokinase). In another embodiment shown on FIG. 1, when the second metabolic product is ethanol, the bacterial host cell can be selected based on its ability to convert pyruvate into acetaldehyde (via the activity or one or more pyruvate decarboxylase). In some embodiments, the bacterial host cell is genetically modified to provide or increase its ability to convert pyruvate into acetaldehyde (via the activity or one or more pyruvate decarboxylase). In yet another embodiment shown on FIG. 1, when the second metabolic product is ethanol, the bacterial host cell can be selected based on its ability to convert acetaldehyde into ethanol (via the activity or one or more alcohol dehydrogenase). In some embodiments, the bacterial host cell is genetically modified to provide or increase its ability to convert acetaldehyde into ethanol (via the activity or one or more alcohol dehydrogenase).

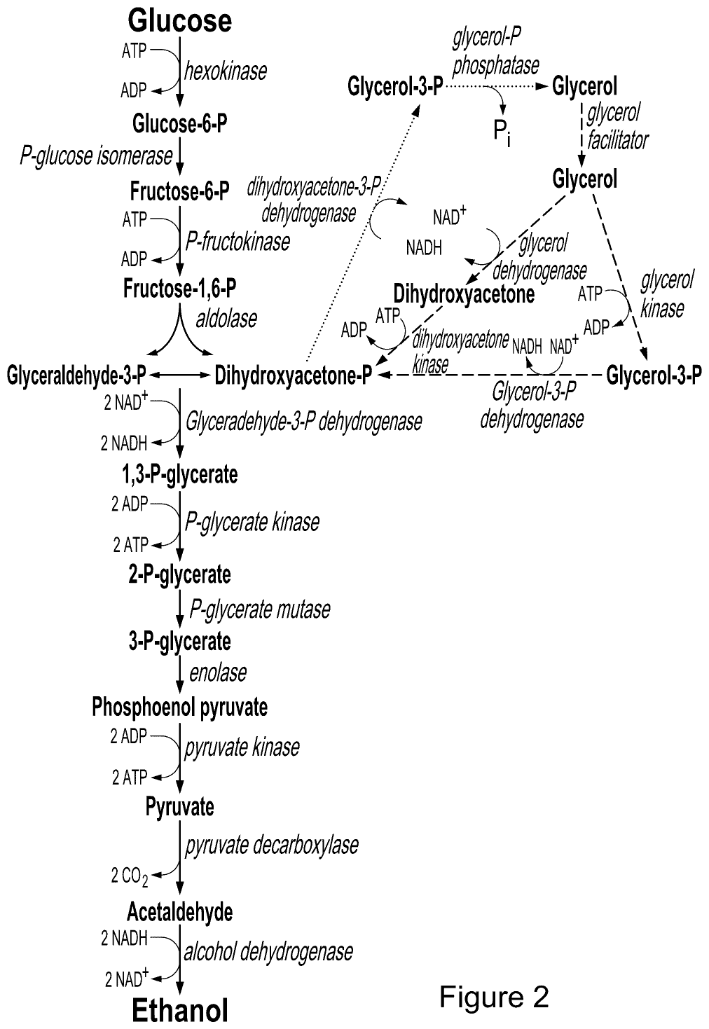

In another embodiment, shown on FIG. 2, the first metabolic product produced by the yeast host cell can be glycerol which can subsequently be metabolized to ethanol production (e.g., the second metabolic product) by the bacterial host cell. In such embodiment, the yeast host cell can be selected based on its ability to convert dihydroxyacetone-P into glycerol-3-phosphate (glycerol-3-P, via the activity of one or more dihydroxyacetone-3-P dehydrogenase), glycerol-3-P into glycerol (via the activity of one or more a glycerol-3-P phosphatase). In some embodiments, the yeast host cell can be genetically modified to provide or increase its ability to convert dihydroxyacetane-P into glycerol-3-phosphate (glycerol-3-P, via the activity of one or more dihydroxyacetone-3-P dehydrogenase) and/or glycerol-3-P into glycerol (via the activity of one or more a glycerol-3-P phosphatase). In the embodiment shown on FIG. 2, the bacterial host cell can be selected based on its ability to import glycerol (via the activity or one or more glycerol facilitator), to convert glycerol into glycerol-3-P (via the activity of one or more glycerol kinase), glycerol-3-P into dihydroxyacetone-P (via the activity of one or more glycerol-3-P dehydrogenase), glycerol into dihydroxyacetone (via the activity of one or more glycerol dehydrogenase) and dihydroxyacetone into dihydroxyacetone-P (via the activity or one or more dihydroxyacetone kinase). In some embodiments, the bacterial host cell is genetically modified to provide or increase its ability to import glycerol (via the activity or one or more glycerol facilitator), to convert glycerol into glycerol-3-P (via the activity of one or more glycerol kinase), glycerol-3-P into dihydroxyacetone-P (via the activity of one or more glycerol-3-P dehydrogenase), glycerol into dihydroxyacetone (via the activity of one or more glycerol dehydrogenase) and/or dihydroxyacetone into dihydroxyacetone-P (via the activity or one or more dihydroxyacetone kinase).

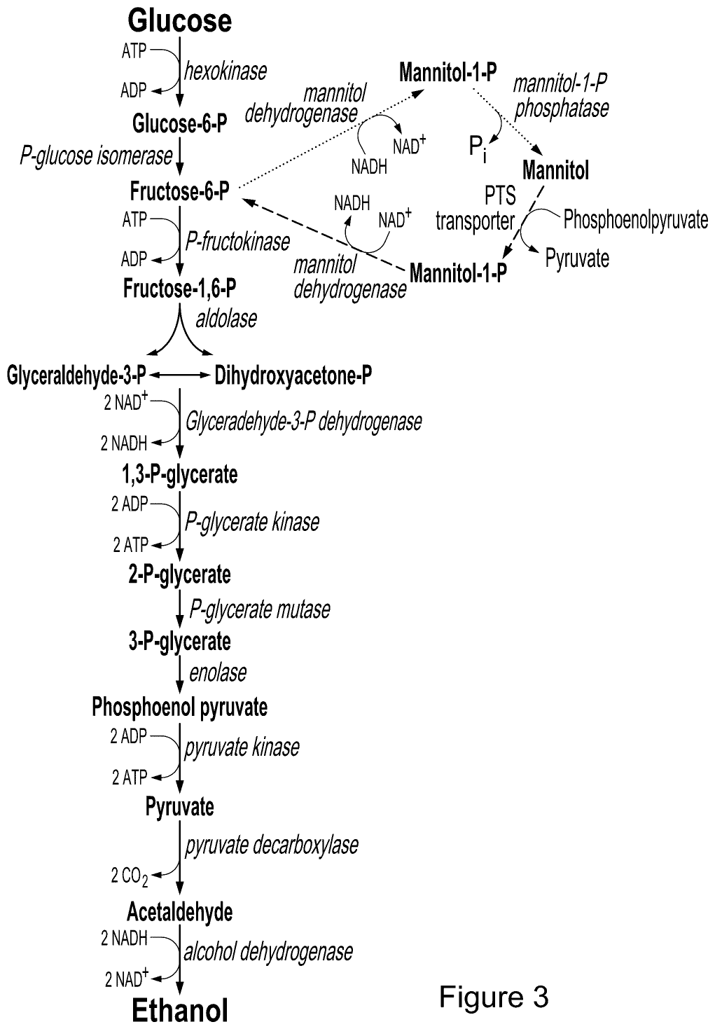

In another embodiment, shown on FIG. 3, the first metabolic product produced by the yeast host cell can be mannitol which can subsequently be metabolized to ethanol (e.g., the second metabolic product) by the bacterial host cell. In such embodiment, the yeast host cell can be selected based on its ability to convert fructose-6-P into mannitol-1-phosphate (mannitol-1-P, via the activity of one or more mannitol dehydrogenase) and mannitol-1-P into mannitol (via the activity of one or more a mannitol-1-P phosphatase). In some embodiments, the yeast host cell can be genetically modified to provide or increase its ability to convert fructose-6-P into mannitol-1-phosphate (mannitol-1-P, via the activity of one or more mannitol dehydrogenase) and/or mannitol-1-P into mannitol (via the activity of one or more a mannitol-1-P phosphatase). In the embodiment shown on FIG. 3, the bacterial host cell can be selected based on its ability to convert mannitol into mannitol-1-phosphate (mannitol-1-P, via the activity of one or more PTS transporter) and mannitol-1-P into fructose-6-P (via the activity of one or more mannitol dehydrogenase). In some embodiments, the bacterial host cell is genetically modified to provide or increase its ability to convert mannitol into mannitol-1-phosphate (mannitol-1-P, via the activity of one or more PTS transporter) and/or mannitol-1-P into fructose-6-P (via the activity of one or more mannitol dehydrogenase).

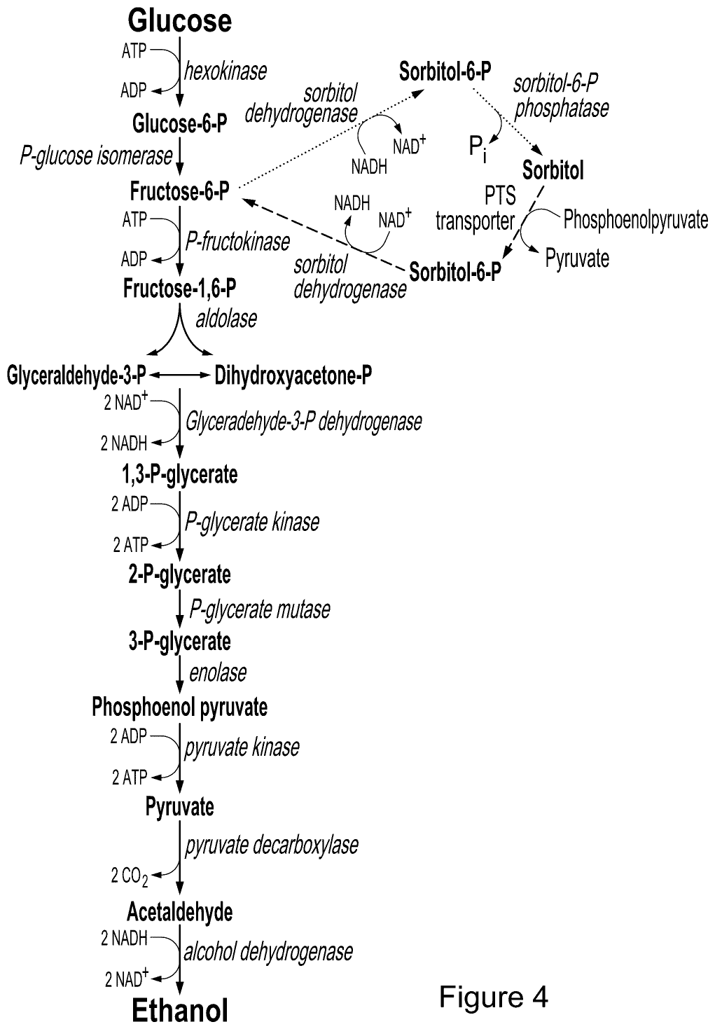

In another embodiment, shown on FIG. 4, the first metabolic product produced by the yeast host cell can be sorbitol which can subsequently be metabolized to ethanol (e.g., the second metabolic product) by the bacterial host cell. In such embodiment, the yeast host cell can be selected based on its ability to convert fructose-6-P into sorbitol-6-phosphate (sorbitol-6-P, via the activity of one or more sorbitol dehydrogenase) and sorbitol-6-P into sorbitol (via the activity of one or more a sorbitol-6-P phosphatase). In some embodiments, the yeast host cell can be genetically modified to provide or increase its ability to convert fructose-6-P into sorbitol-6-phosphate (sorbitol-6-P, via the activity of one or more sorbitol dehydrogenase) and/or sorbitol-6-P into sorbitol (via the activity of one or more a sorbitol-6-P phosphatase). In the embodiment shown on FIG. 4, the bacterial host cell can be selected based on its ability to convert sorbitol into sorbitol-6-phosphate (sorbitol-6-P, via the activity of one or more PTS transporter) and sorbitol-6-P into fructose-6-P (via the activity of one or more sorbitol dehydrogenase). In some embodiments, the bacterial host cell is genetically modified to provide or increase its ability to convert sorbitol into sorbitol-6-phosphate (sorbitol-6-P, via the activity of one or more PTS transporter) and/or sorbitol-6-P into fructose-6-P (via the activity of one or more sorbitol dehydrogenase).

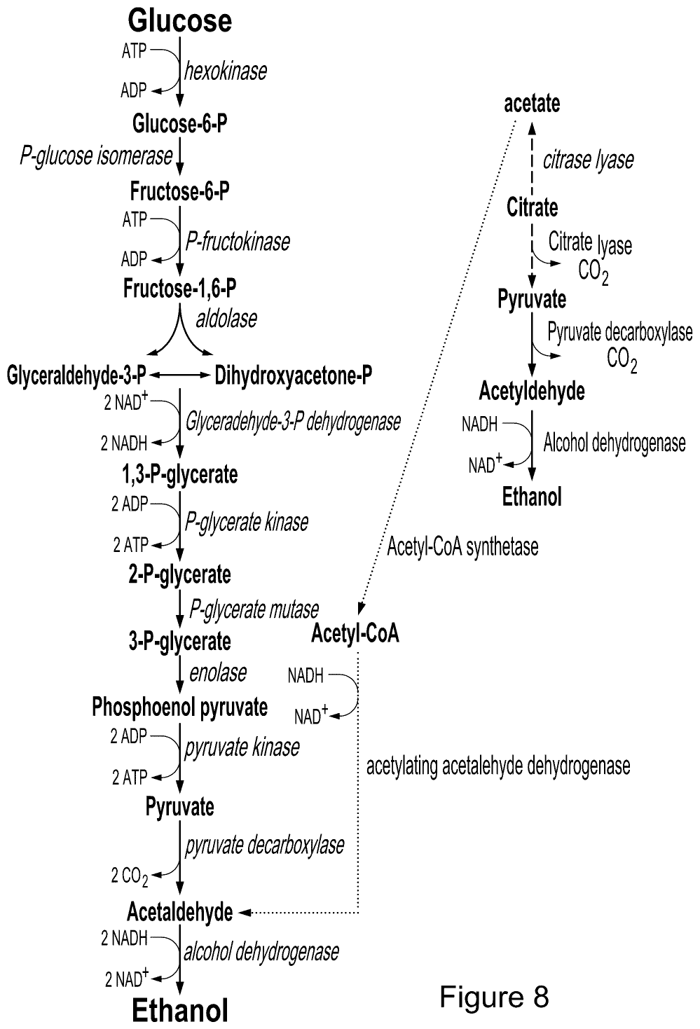

In a further embodiment, shown on FIG. 8, the first metabolic product produced by the bacterial host cell can be acetic acid (or acetate) which can subsequently be metabolized to ethanol (e.g., the second metabolic product) by the yeast host cell. In the embodiment shown on FIG. 8, the bacterial host cell is capable of producing acetate which can further be hydrolyzed into acetic acid in subsequent steps. Still in the embodiments show on FIG. 8, the bacterial host cell can be selected based on its ability to convert citric acid (or its associated esther citrate) into acetic acid (or its associated esther acetate) (via the activity of one or more citrate lyase). In some embodiments, the bacterial host cell can be genetically modified to provide or increase its ability to convert citric acid (citrate) into acetic acid (acetate) (via the activity of one or more citrate lyase). In the embodiment shown on FIG. 8, the yeast host cell can be selected based on its ability to convert acetic acid (acetate) into acetyl-CoA, via the activity of one or more acetyl-CoA synthetase (such as for example ACS2). In some embodiments, the yeast host cell is genetically modified to provide or increase its ability to convert acetic acid (acetate) into acetyl-coA, via the activity of one or more acetyl-coA synthetase (such as for example ACS2). Still in the embodiment shown on FIG. 8, the yeast host cell can be selected based on its ability to convert acetyl-coA into acetaldehyde, via the activity of one or more bifunctional acetylating aldehyde dehydrogenase/alcohol dehydrogenase (such as for example ADHE). In some embodiments, the yeast host cell is genetically modified to provide or increase its ability to convert acetyl-coA into acetaldehyde, via the activity of one or more bifunctional acetylating aldehyde dehydrogenase/alcohol dehydrogenase (such as for example ADHE).

The combination of the present disclosure comprises a recombinant yeast host cell and/or a recombinant bacterial host cells. These recombinant host cells can be obtained by introducing one or more genetic modifications in a corresponding native (parental) yeast/bacterial host cell. When the genetic modification is aimed at reducing or inhibiting the expression of a specific targeted gene (which is endogenous to the host cell), the genetic modifications can be made in one or both copies of the targeted gene(s). When the genetic modification is aimed at increasing the expression of a specific targeted gene, the genetic modification can be made in one or multiple genetic locations. In the context of the present disclosure, when recombinant yeast and bacterial host cells are qualified as being “genetically engineered”, it is understood to mean that they have been manipulated to either add at least one or more heterologous or exogenous nucleic acid residue and/or removed at least one endogenous (or native) nucleic acid residue. In some embodiments, the one or more nucleic acid residues that are added can be derived from an heterologous cell or the recombinant host cell itself. In the latter scenario, the nucleic acid residue(s) is (are) added at a genomic location which is different than the native genomic location. The genetic manipulations did not occur in nature and are the results of in vitro manipulations of the native yeast or bacterial host cell.

When expressed in recombinant host cells, the polypeptides (including the enzymes) described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When an heterologous nucleic acid molecule is present in the recombinant host cell, it can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the host cell's genome. In such embodiment, the nucleic acid molecule can be stable and self-replicating.

In some embodiments, heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

In some embodiments, heterologous nucleic acid molecules which can be introduced into the recombinant host cells are codon-optimized with respect to the intended recipient recombinant yeast host cell so as to limit or prevent homologous recombination with the corresponding native gene.

The heterologous nucleic acid molecules of the present disclosure comprise a coding region for the one or more enzymes to be expressed by the host cell. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region.

Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

In the heterologous nucleic acid molecule described herein, the promoter and the nucleic acid molecule coding for the one or more enzymes can be operatively linked to one another. In the context of the present disclosure, the expressions “operatively linked” or “operatively associated” refers to fact that the promoter is physically associated to the nucleotide acid molecule coding for the one or more enzyme in a manner that allows, under certain conditions, for expression of the one or more enzyme from the nucleic acid molecule. In an embodiment, the promoter can be located upstream (5′) of the nucleic acid sequence coding for the one or more enzyme. In still another embodiment, the promoter can be located downstream (3′) of the nucleic acid sequence coding for the one or more enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more enzyme. The promoters can be located, in view of the nucleic acid molecule coding for the one or more protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be heterologous to the nucleic acid molecule encoding the one or more enzymes. The promoter can be heterologous or derived from a strain being from the same genus or species as the host cell. In an embodiment, the promoter is derived from the same genus or species of the yeast host cell and the heterologous polypeptide is derived from different genus that the host cell.

In some embodiments, the present disclosure concerns the expression of one or more heterologous enzyme, a variant thereof or a fragment thereof in a host cell. The enzyme “variants” have at least 50%, 55%, 60%, 65%, 70%, 75%, 80%, 85%, 90%, 95%, 96%, 97%, 98% or 99% identity to the heterologous enzymes described herein and exhibits the biological activity associated with the heterologous enzyme. In an embodiment, the variant enzyme exhibits at least 50%, 55%, 60%, 65%, 70%, 75%, 80%, 85%, 90%, 95%, 96%, 97%, 98% or 99% of the biological activity of the wild-type heterologous enzyme. A variant comprises at least one amino acid difference when compared to the amino acid sequence of the native enzyme. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED-5. The variant heterologous enzymes described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the enzyme can be a conservative variant or an allelic variant. As used herein, a conservative variant refers to alterations in the amino acid sequence that do not adversely affect the biological functions of the enzyme. A substitution, insertion or deletion is said to adversely affect the protein when the altered sequence prevents or disrupts a biological function associated with the enzyme. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the enzyme.

The heterologous enzyme can be a fragment of an enzyme or fragment of a variant of an enzyme which exhibits the biological activity of the heterologous enzyme or the variant. In an embodiment, the fragment enzyme exhibits at least 50%, 55%, 60%, 65%, 70%, 75%, 80%, 85%, 90%, 95%, 96%, 97%, 98% or 99% of the biological activity of the heterologous enzyme or variant thereof. Enzyme “fragments” have at least at least 100, 200, 300, 400, 500 or more consecutive amino acids of the enzyme or the enzyme variant. A fragment comprises at least one less amino acid residue when compared to the amino acid sequence of the enzyme and still possess the enzymatic activity of the full-length enzyme. In some embodiments, the “fragments” have at least 50%, 55%, 60%, 65%, 70%, 75%, 80%, 85%, 90%, 95%, 96%, 97%, 98% or 99% identity to the enzymes described herein. In some embodiments, fragments of the enzymes can be employed for producing the corresponding full-length enzyme by peptide synthesis. Therefore, the fragments can be employed as intermediates for producing the full-length proteins.

In some additional embodiments, the present disclosure also provides expressing a protein encoded by a gene ortholog of a gene known to encode an enzyme. A “gene ortholog” is understood to be a gene in a different species that evolved from a common ancestral gene by speciation. In the context of the present invention, a gene ortholog encodes an enzyme exhibiting the same biological function than the native enzyme.

In some further embodiments, the present disclosure also provides expressing a protein encoded by a gene paralog of a gene known to encode an enzyme. A “gene paralog” is understood to be a gene related by duplication within the genome. In the context of the present invention, a gene paralog encodes an enzyme that could exhibit additional biological function than the native enzyme.

### Yeast Host Cell

In the context of the present disclosure, the combination comprises a yeast host cell which can, in some embodiments, be recombinant. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the yeast host cell is from the genus Saccharomyces and, in some embodiments, from the species Saccharomyces cerevisiae.

The yeast host cell of the present disclosure can have a first metabolic pathway comprising one or more enzymes for producing a first metabolic product. The yeast host cell can have the intrinsic ability to produce the first metabolic product or can be engineered to have increased activity in one or more first enzymes in the first metabolic pathway. The increased in activity can be caused at least in part by introducing of one or more first genetic modifications in a native yeast host cell to obtain the recombinant yeast host cell. As such, the activity of the one or more first enzymes of the recombinant yeast host cell is considered “increased” because it is higher than the activity of the one or more first enzymes in the native yeast host cell (e.g., prior to the introduction of the one or more first genetic modifications). The one or more first genetic modifications is not limited to a specific modification provided that it does increase the activity, and in some embodiments, the expression of the one or more first enzymes. For example, the one or more first genetic modifications can include the addition of a promoter to increase the expression of the one or more (endogenous) first enzymes. Alternatively or in addition, the one or more first genetic modifications can include the introduction of one or more copies of a gene(s) encoding the one or more first (heterologous) enzymes in the recombinant yeast host cell.

In an embodiment, the first metabolic product is a carbohydrate and the yeast host cell has the ability to produce the carbohydrate or has increased activity in one or more first enzymes for producing the carbohydrate. In some embodiments, the first metabolic product is a carbohydrate which is not substantially metabolized by the yeast host cell. For example, the first metabolic product can be a pentose sugars or sugar polymers with a degree of polymerization of 2, 3, 4, or more. Exemplary sugars not naturally or not preferentially utilized by yeasts include, but are not limited to, xylose, arabinose, trehalose, maltose, isomaltose, cellobiose, cellobiotriose, maltotriose, isomaltotriose, panose, raffinose, stachyose, maltotetraose, and maltodextrin. In another embodiment, the first metabolic product can be a sugar alcohol, a 2- to 24-carbon chain including at least one alcohol moiety. Sugar alcohols include, but are not limited to, ethylene glycol, glycerol, erythritol, threitol, arabitol, xylitol, ribitol, mannitol, sorbitol, galactitol, fucitol, iditol, inositol, volemitol, isomalt, maltitol, lactitol, maltotriitol, maltotetraitol or polyglycitol. In still another embodiment, the first metabolic product can be an protectant for the yeast host cell, e.g. it has the ability to protect, at least in part, the yeast host of cell from a stressor (lactic acid, formic acid, bacterial contamination, etc.).

In a specific embodiment, the first metabolic product is trehalose. In such specific embodiment, the yeast host cell can have increased biological activity in at least one of a trehalose-6-phosphate (trehalose-6-P) synthase or a trehalose-6-phosphate phosphastase or both enzymes. As indicated above, this can be done by introducing a strong and/or constitutive promoter to increase the expression of the endogenous trehalose-6-P synthase and/or the endogenous trehalose-6-P phosphatase. Alternatively or in combination, this can also be done by introducing at least one copy of one or more heterologous nucleic acid molecules encoding an heterologous trehalose-6-P synthase and/or an heterologous trehalose-6-P phosphatase. In an embodiment, the yeast host cell has increased biological activity of a trehalose-6-P synthase, but not of the trehalose-6-P phosphatase. In another embodiment, the yeast host cell has increased biological activity of a trehalose-6-P phosphatase, but not of the trehalose-6-P synthase. In still another embodiment, the yeast host cell has increased biological activity in both a trehalose-6-P synthase and a trehalose-6-P phosphatase.

As used herein, the term “trehalose-6-phosphate synthase” refers to an enzyme capable of catalyzing the conversion of glucose-6-phosphate and UDP-D-glucose to α-α-trehalose-6-phosphate and UDP. In Saccharomyces cerevisiae, the trehalose-6-phosphate synthase gene can be referred to TPS1 (SGD: S000000330, Gene ID: 852423), BYP1, CIF1, FDP1, GGS1, GLC6 or TSS1. The yeast host cell of the present disclosure can include a native gene encoding for the trehalose-6-phosphate synthase and/or an heterologous nucleic acid molecule coding for TPS1, a variant thereof, a fragment thereof or for a protein encoded by a tps1 gene ortholog. In some embodiments, the yeast host cell has an heterologous nucleic acid sequence for the expression of the amino acid sequence of SEQ ID NO: 9, a variant of SEQ ID NO: 9 or a fragment of SEQ ID NO: 9.

As also used herein, the term “trehalose-6-phosphate phosphatase” refers to an enzyme capable of catalyzing the conversion of α-α-trehalose-6-phosphate and H₂O into phosphate and trehalose. In Saccharomyces cerevisiae, the trehalose-6-phosphate phosphatase gene can be referred to TPS2 (SGD: S000002481, Gene ID: 851646), HOG2 or PFK3. The yeast host cell of the present disclosure can include a native gene encoding for the trehalose-6-phosphate phosphatase and/or a nucleic acid molecule coding for TPS2, a variant thereof, a fragment thereof or for a protein encoded by a tps2 gene ortholog. In some embodiments, the yeast host cell has an heterologous nucleic acid sequence for the expression of the amino acid sequence of SEQ ID NO: 10, a variant of SEQ ID NO: 10 or a fragment of SEQ ID NO: 10.

Alternatively or in combination, the yeast host cell has increased biological activity in a protein involved in regulating trehalose production. As indicated above, this can be done by introducing a strong and/or constitutive promoter to increase the expression of the endogenous protein involved in regulating trehalose production. Alternatively or in combination, this can also be done by introducing at least one copy of one or more heterologous nucleic acid molecules encoding a protein involved in regulating trehalose production.

As used herein, the term “protein involved in regulating trehalose production” refers to a protein capable of modulating the activity of enzymes involved in the production of trehalose. In Saccharomyces cerevisiae, proteins involved in regulating trehalose production include, but are not limited to a subunit of the trehalose 6-phosphate synthase/phosphatase TPS3 and trehalose synthase long chain (TSL1).

In some specific embodiments, the protein involved in regulating trehalose production is TSL1. The yeast host cell of the present disclosure can include a native TSL1 protein and/or express an heterologous TSL1 (as well as a variant or a fragment thereof) from any origin including, but not limited to Saccharomyces cerevisiae (SGD: S000004566, Gene ID 854872), Gallus gallus (Gene ID107050801), Kluyveromyces marxianus (Gene ID: 34714558), Saccharomyces eubayanus (Gene ID: 28933129), Schizosaccharomyces japonicus (Gene ID: 7049746), Pichia kudriavzevii (Gene ID: 31691677) or Hydra vulgaris (Gene ID 105848257).

In some additional embodiments (which may be an alternative or a combination to the previous embodiment), the protein involved in regulating trehalose production is TPS3. The yeast host cell of the present disclosure can including a native TPS3 polypeptide and/or express an heterologous TPS3 (as well as a variant or a fragment thereof) from any origin including, but not limited to Saccharomyces cerevisiae (SGD: S000004874, Gene ID: 855303), Arabidopsis thaliana (Gene ID: 838270), Sugiyamaella lignohabitans (Gene ID: 30034940), Candida albicans (Gene ID: 3641205), Chlamydomonas reinhardtii (Gene ID: 5717648), Candida orthopsilosis (Gene ID: 14539600), Isaria fumosorosea (Gene ID: 30022220), Penicillium digitatum (Gene ID: 26236600), Cordyceps militaris (Gene ID: 18168860), Aspergillus fumigatus (Gene ID: 3506432), Aspergillus flavus (Gene ID: 7918663), Aspergillus clavatus (Gene ID: 4705657), Aspergillus fischeri (Gene ID: 4588220), Aspergillus vadensis (Gene ID 37209217), Aspergillus costaricaensis (Gene ID: 37185236), Aspergillus piperis (Gene ID: 37160157), Aspergillus aculeatinus (Gene ID: 37150689), Aspergillus neoniger (Gene ID: 37124414), Aspergillus sclerotioniger (Gene ID: 37114541), Aspergillus brunneoviolaceus (Gene ID: 37089207), Aspergillus saccharolyticus (Gene ID: 37076724), Aspergillus eucalypticola (Gene ID: 37051636), Aspergillus novofumigatus (Gene ID: 36535454), Verticillium dahliae (Gene ID: 20704316), Trichophyton rubrum (Gene ID: 10373473), Nannizzia gypsea (Gene ID: 10027518), Verticillium alfalfae (Gene ID: 9532751), Ajellomyces dermatitidis (Gene ID: 8508720), Talaromyces stipitatus (Gene ID: 8104915) or Talaromyces marneffei (Gene ID: 7024067).

In some embodiments, especially when the metabolism of the first metabolic product is oxidative (for example when it is mannitol, sorbitol or glycerol), the present disclosure provides a yeast host cell which can be genetically modified to provide a secondary substrate to the bacterial host cell which could act as an electron acceptor and allow redox balance. This can be done, for example, by introducing one or more heterologous nucleic acid molecules encoding a NADP⁺-dependent aldehyde dehydrogenase and/or a phosphoketolase. This can also be done by introducing a strong promoter upstream of the native NADP⁺-dependent aldehyde dehydrogenase and/or phosphoketolase to increase its level of expression. Alternatively or in combination, this can be done by introducing at least one copy of one or more heterologous nucleic acid molecules encoding a protein having NADP⁺-dependent aldehyde dehydrogenase and/or phosphoketolase activity. The adjustment of the redox balance can also be done, alternatively or in combination, by supplementing the fermentation medium with an electron acceptor, such as, for example acetate.

As used in the context of the present disclosure, the NADP⁺-dependent aldehyde dehydrogenase is an enzyme that catalyzes the conversion of an aldehyde, NADP+ and water into an acid, NADPH and an hydrogen atom (E.C. 1.2.1.4). In an embodiment, the NADP⁺-dependent aldehyde dehydrogenase can be derived from S. cerevisiae ALD6 (Gene ID: 856044), Candida albicans ALD6 (Gene ID: 3647407), Kluyveromyces marxianus ALD6 (Gene ID: 34714396) or Candida orthopsilosis (Gene ID: 14538090).

As used in the context of the present disclosure, the phosphoketolase (PHK) is an enzyme that catalyzes D-xylulose 5-phosphate and phosphate into acetyl phosphate, D-glyceraldehyde 3-phosphate and water (E.C. 4.1.2.9 and 4.1.2.22). In some embodiments, PHK is up-regulated. In some embodiments, single-specificity phosphoketolase is up-regulated. In some embodiments, dual-specificity phosphoketolase is up-regulated. In some embodiments, the PHK is derived from a genus selected from the group consisting of Aspergillus, Neurospora, Lactobacillus, Bifidobacterium, and Penicillium. In some embodiments, the PHK is from Bifidobacterium adolescentis. In some embodiments the PHK is from Aspergillus niger. In some embodiments, the PHK is from Neurospora crassa. In some embodiments, the PHK is from Lactobacillus paracasei. In some embodiments, the PHK is from Lactobacillus plantarum.

In another specific embodiment, the first metabolic product is a carbohydrate, which is a sugar alcohol and in some specific embodiments, the carbohydrate is mannitol. In such embodiment, the yeast host cell can have native mannitol dehydrogenase activity and/or be genetically modified to increased mannitol dehydrogenase activity. In an embodiment, the mannitol dehydrogenase activity is provided by the enzyme mannitol-1-phosphate 5-dehydrogenase catalyzes the conversion of fructose-6-phosphate and NADH into mannitol-1-phosphate and NAD+ (EC 1.1.1.17). Mannitol-1-phosphate can then be converted to mannitol via the promiscuous phosphatase activity of the yeast host cell. Alternatively or in combination, the yeast host cell can have native mannitol 1-phosphate phosphatase activity and/or can be engineered to provide or increase mannitol 1-phosphate phosphatase activity. As indicated above, the increase in mannitol-1-phosphate 5-dehydrogenase activity can be done by introducing a strong and/or constitutive promoter to increase the expression of the endogenous mannitol-1-phosphate 5-dehydrogenase. Alternatively or in combination, this can also be done by introducing at least one copy of one or more heterologous nucleic acid molecules encoding mannitol-1-phosphate 5-dehydrogenase. The mannitol-1-phosphate 5-dehydrogenase can be derived from the mtlD gene. The mtlD gene encoding the mannitol-1-phosphate 5-dehydrogenase can be of yeast or bacterial origin. In some embodiments, the mtlD is derived from a genus selected from the group consisting of Escherichia, Aspergillus, Neurospora, Lactobacillus, Bifidobacterium, Lactococcus, Bacillus, and Acinetobacter. In some embodiments, mtlD is up-regulated. In some embodiments, the mtlD is from Escherichia coli. In some embodiments the mtlD is from Lactobacillus paracasei. In some embodiments, the mtlD is from Lactobacillus plantarum. In some embodiments, the mtlD is from Lactococcus lactis. In some embodiments, the mtlD is from Bacillus subtilis. In some embodiments the mtlD is from Pseudomonas sp. In some embodiments the mtlD is from Acinetobacter baylyi. In some embodiments the mtlD is from Aspergillus niger. In an embodiment, the MTLD polypeptide is from Escherichia sp., such as, for example Escherichia coli. In such embodiment, the MTLD polypeptide can have, for example, the amino acid sequence of SEQ ID NO: 27, be a variant of the amino acid sequence of SEQ ID NO: 27 or be a fragment of the amino acid sequence of SEQ ID NO: 27 or a variant thereof. In an embodiment, the MTLD polypeptide is encoded by an heterologous nucleic acid molecule having the nucleic acid sequence of SEQ ID NO: 28, being a variant of the nucleic acid sequence of SEQ ID NO: 28 or being a fragment of the nucleic acid sequence or SEQ ID NO: 28 or a variant thereof. In an embodiment, the MTLD2 polypeptide is from Lactobacillus sp., such as, for example Lactobacillus paracasei. In such embodiment, the MTLD2 polypeptide can have, for example, the amino acid sequence of SEQ ID NO: 39, be a variant of the amino acid sequence of SEQ ID NO: 39 or be a fragment of the amino acid sequence of SEQ ID NO: 39 or a variant thereof. In an embodiment, the MTLD2 polypeptide is encoded by an heterologous nucleic acid molecule having the nucleic acid sequence of SEQ ID NO: 40, being a variant of the nucleic acid sequence of SEQ ID NO: 40 or being a fragment of the nucleic acid sequence or SEQ ID NO: 40 or a variant thereof.

In another specific embodiment, the carbohydrate is a sugar alcohol and in some specific embodiments, the carbohydrate is sorbitol. In such embodiment, the yeast host cell can have native sorbitol dehydrogenase activity and/or can be modified to provide or increase sorbitol dehydrogenase activity. In an embodiment, the sorbitol dehydrogenase activity is provided by the enzyme sorbitol-6-phosphate 2-dehydrogenase which catalyzes the conversion of fructose-6-phosphate and NADH into sorbitol 6-phosphate and NAD+ (EC 1.1.1.140). Sorbitol 6-phosphate can then be converted to sorbitol via the promiscuous phosphatase activity of the yeast host cell. Alternatively or in combination, the yeast host cell can have native sorbitol-6-phosphate phosphatase activity and/or be genetically modified to provide or increase sorbitol-6-phosphate phosphatase activity. As indicated above, the increase in sorbitol 6-phosphate 2-dehydrogenase activity can be done by introducing a strong and/or constitutive promoter to increase the expression of the endogenous sorbitol 6-phosphate 2-dehydrogenase. Alternatively or in combination, this can also be done by introducing at least one copy of one or more heterologous nucleic acid molecules encoding sorbitol 6-phosphate 2-dehydrogenase. The gene encoding the sorbitol 6-phosphate 2-dehydrogenase can be of yeast or bacterial origin. In an embodiment, the sorbitol 6-phosphate 2-dehydrogenase can be encoded by the srlD gene. In some embodiments, the srlD is derived from a genus selected from the group consisting of Escherichia, Lactobacillus, Clostridium, Streptococcus, and Klebsiella. In some embodiments, the srlD gene is up-regulated. In some embodiments, the srlD gene is from Escherichia coli. In some embodiments the srlD gene is from Lactobacillus paracasei. In some embodiments, the srlD gene is from Lactobacillus plantarum. In some embodiments the srlD gene is from Clostridium pasteurianum. In some embodiments the srlD gene is from Klebsiella aerogenes. The gene encoding the sorbitol 6-phosphate dehydrogenase can be derived from the srlD gene and can be, without limitations, from the following sources: Escherichia coli (Gene ID: 948937), Clostridioides difficile (4915542), Mycoplasma mycoides subsp. mycoides (Gene ID: 2744550), Clostridium botulinum (Gene ID: 5399122), Shigella dysenteriae (Gene ID: 3796629), Shigella flexneri (Gene ID: 1027455), Escherichia coli (Gene ID: 7152897 or 7157974), Salmonella enterica subsp. enterica (Gene ID: 1254358 or 1249263), Clostridium botulinum (Gene ID: 5187667) or Saccharomyces cerevisiae (Gene IDs: 851539 and 854095). In an embodiment, the SRLD polypeptide is from Escherichia sp., such as, for example Escherichia coli. In such embodiment, the SRLD polyppeptide can have, for example, the amino acid sequence of SEQ ID NO: 29, be a variant of the amino acid sequence of SEQ ID NO: 29 or be a fragment of the amino acid sequence of SEQ ID NO: 29 or a variant thereof. In an embodiment, the SRLD polypeptide is encoded by an heterologous nucleic acid molecule having the nucleic acid sequence of SEQ ID NO: 30, being a variant of the nucleic acid sequence of SEQ ID NO: 30 or being a fragment of the nucleic acid sequence or SEQ ID NO: 30 or a variant thereof.

In another specific embodiment, the carbohydrate is a sugar alcohol and in some specific embodiments, the carbohydrate is glycerol. In such embodiment, the yeast host cell does not need to be genetically modified as it has the intrinsic ability to produce glycerol. Alternatively, the yeast host cell can be genetically modified to increase dihydrogenaseacetone-3-phosphate dehydrogenase activity and/or glycerol-phosphate phosphatase activity.

The yeast host cell of the present disclosure can have a second metabolic pathway comprising one or more enzymes for producing a second metabolic product. The yeast host cell can have the intrinsic ability to produce the second metabolic product or can be engineered to have increased activity in one or more second enzymes in the second metabolic pathway. The increased in activity can be caused at least in part to the introduction of one or more second genetic modifications in a native yeast host cell to obtain the recombinant yeast host cell. As such, the activity of the one or more second enzymes of the recombinant yeast host cell is considered “increased” because it is higher than the activity of the one or more second enzymes in the native yeast host cell (e.g., prior to the introduction of the one or more second genetic modifications). The one or more second genetic modifications is not limited to a specific modification provided that it does increase the activity, and in some embodiments, the expression of the one or more second enzymes. For example, the one or more second genetic modifications can include the addition of a promoter to increase the expression of the one or more (endogenous) second enzymes. Alternatively or in addition, the one or more second genetic modifications can include the introduction of one or more copies of a gene(s) encoding the one or more second (heterologous) enzymes in the recombinant yeast host cell.

In an embodiment, the second metabolic product is ethanol and the yeast host cell has the ability to produce the ethanol from the organic acid (or associated ester) or has increased activity in one or more second enzymes for converting the organic acid into ethanol. In an embodiment, the organic acid can be, without limitation, acetic acid. As used in the context of the present disclosure, the expression “organic acid” includes associated organic esthers which can be hydrolyzed into the organic acid. An embodiment of an organic acid is acetic acid and an embodiment of a corresponding organic esther is acetate.

In a specific embodiment in which the yeast host cell is capable of converting the organic acid (or associated esther) into ethanol, the yeast host cell can have increased biological activity in a polypeptide having acetylating aldehyde dehydrogenase activity. As used in the present disclosure, a polypeptide having acetylating aldehyde dehydrogenase activity has the ability to convert acetyl-coA into an aldehyde. In some embodiments, the polypeptide having acetylating aldehyde dehydrogenase activity is an AADH or is a bifunctional acetylating aldehyde dehydrogenase/alcohol dehydrogenase (ADHE). The bifunctional acetaldehyde/alcohol dehydrogenase is an enzyme capable of converting acetyl-CoA into acetaldehyde as well as acetaldehyde into ethanol. Heterologous bifunctional acetaldehyde/alcohol dehydrogenases (AADH) include but are not limited to those described in U.S. Pat. No. 8,956,851 and WO 2015/023989. Heterologous AADHs of the present disclosure include, but are not limited to, the ADHE polypeptides or a polypeptide encoded by an adhe gene ortholog. In an embodiment, the AADH is from a Bifidobacterium sp., such as for example, a Bifidobacterium adolescentis. In an embodiment, the AADH has the amino acid sequence of SEQ ID NO: 15 or 47, is a variant of the amino acid sequence of SEQ ID NO: 15 or 47 or is a fragment of the amino acid sequence of SEQ ID NO: 15 or 47. In such embodiment, the genetic modification can comprise introducing an heterologous nucleic acid molecule (which can have, in some embodiments, the nucleic acid sequence of SEQ ID NO: 48) encoding a protein having the amino acid sequence of SEQ ID NO: 15 or 47, being a variant of the amino acid sequence of SEQ ID NO: 15 or 47 or being a fragment of the amino acid sequence of SEQ ID NO: 15 or 47. In a specific embodiment in which the yeast host cell is capable of converting the organic acid (such as, for example acetic acid or its associated esther acetate) into ethanol, the yeast host cell can have increased biological activity in an acetyl-coA synthetase. The acetyl-coA synthase is an enzyme capable of converting acetic acid into acetyl-CoA. Heterologous acetyl-coA synthetase include but are not limited to GenBank Accession number CAA97725. Heterologous acetyl-coA synthetase of the present disclosure include, but are not limited to, the ACS2 polypeptides or a polypeptide encoded by an acs2 gene ortholog. In an embodiment, the AADH (e.g., ACS2) is from a Saccharomyces sp., such as for example, a Saccharomyces cerevisiae. In an embodiment, the acetyl-coA synthetase has the amino acid sequence of SEQ ID NO: 49, is a variant of the amino acid sequence of SEQ ID NO: 49 or is a fragment of the amino acid sequence of SEQ ID NO: 49. In such embodiment, the genetic modification can comprise introducing an heterologous nucleic acid molecule encoding a protein having the amino acid sequence of SEQ ID NO: 50, being a variant of the amino acid sequence of SEQ ID NO: 50 or being a fragment of the amino acid sequence of SEQ ID NO: 50.

In a specific embodiment in which the yeast host cell is capable of converting the organic acid (such as, for example acetic acid or its associated esther acetate) into ethanol, the yeast host cell can have increased biological activity in a NADPH-dependent alcohol dehydrogenase. The protein having NADPH-dependent alcohol dehydrogenase activity can be an ADH polypeptide (for example from Entamoeba sp., including Entamoeba nuttalli (such as, for example, the one having the amino acid sequence of SEQ ID NO: 45), an ADH1 polypeptide variant (e.g., a variant of the amino acid sequence of SEQ ID NO: 45), an ADH1 polypeptide fragment (e.g., a fragment of the amino acid sequence of SEQ ID NO: 45 or a variant thereof) or a polypeptide encoded by an adhl gene ortholog/paralog. In such embodiment, the genetic modification can comprise introducing an heterologous nucleic acid molecule encoding a protein having the amino acid sequence of SEQ ID NO: 46, being a variant of the amino acid sequence of SEQ ID NO: 46 or being a fragment of the amino acid sequence of SEQ ID NO: 46.

In some embodiments, the recombinant yeast host cell can also include one or more additional genetic modifications limiting the production of glycerol. For example, the additional genetic modification can be a genetic modification leading to the reduction in the production, and in an embodiment to the inhibition in the production, of one or more native enzymes that function to produce glycerol. As used in the context of the present disclosure, the expression “reducing the production of one or more native enzymes that function to produce glycerol” refers to a genetic modification which limits or impedes the expression of genes associated with one or more native polypeptides (in some embodiments enzymes) that function to produce glycerol, when compared to a corresponding yeast strain which does not bear such genetic modification. In some instances, the additional genetic modification reduces but still allows the production of one or more native polypeptides that function to produce glycerol. In other instances, the genetic modification inhibits the production of one or more native enzymes that function to produce glycerol. Polypeptides that function to produce glycerol refer to polypeptides which are endogenously found in the recombinant yeast host cell. Native enzymes that function to produce glycerol include, but are not limited to, the GPD1 and the GPD2 polypeptide (also referred to as GPD1 and GPD2, respectively) as well as the GPP1 and the GPP2 polypeptides (also referred to as GPP1 and GPP2, respectively). In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast host cells bearing such genetic modification(s) leading to the reduction in the production of one or more native enzymes that function to produce glycerol are described in WO 2012/138942. In some embodiments, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). In still another embodiment (in combination or alternative to the genetic modification described above).

In yet another embodiment, the recombinant yeast host cell does not bear an additional genetic modification and includes its native genes coding for the GPP/GDP proteins. As such, in some embodiments, there are no genetic modifications leading to the reduction in the production of one or more native enzymes that function to produce glycerol in the recombinant yeast host cell.

Alternatively or in combination, the recombinant yeast host cell can also include one or more additional genetic modifications facilitating the transport of glycerol in the recombinant yeast host cell. For example, the additional genetic modification can be a genetic modification leading to the increase in activity of one or more native enzymes that function to transport glycerol. Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol synthesis. The STL1 protein is natively expressed in yeasts and fungi, therefore the heterologous protein functioning to import glycerol can be derived from yeasts and fungi. STL1 genes encoding the STL1 protein include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 protein is encoded by Saccharomyces cerevisiae Gene ID: 852149. The STL1 protein can have the amino acid sequence of SEQ ID NO: 11 or 53, be a variant of the amino acid sequence of SEQ ID NO: 11 or 53 be a fragment of the amino acid sequence of SEQ ID NO: 11 or 53. In still another embodiment, the STL1 protein can be encoded by an heterologous nucleic acid molecule having the nucleic acid sequence of SEQ ID NO: 54, a variant of the nucleic acid sequence of SEQ ID NO: 54 or a fragment of the nucleic acid sequence of SEQ ID NO: 54. In another embodiment, the STL1 protein is encoded by the heterologous STL1 gene of Pichia sorbitophilia (also referred to as Millerozyma farinose). The STL1 protein can have the amino acid sequence of SEQ ID NO: 51, be a variant of the amino acid sequence of SEQ ID NO: 51 or be a fragment of the amino acid sequence of SEQ ID NO: 51. In still another embodiment, the STL1 protein can be encoded by an heterologous nucleic acid molecule having the nucleic acid sequence of SEQ ID NO: 52, a variant of the nucleic acid sequence of SEQ ID NO: 52 or a fragment of the nucleic acid sequence of SEQ ID NO: 52.

In some embodiments, the yeast host cell can have a further genetic modification allowing the expression of heterologous NADP-specific alcohol dehydrogenase. The presence of this enzyme increases the availability of cytosolic NADH, by creating a redox imbalance between glycolysis and ethanol fermentation, and increases acetate conversion in the yeast host cell. In an embodiment, the NADP-specific alcohol dehydrogenase is from Entamoeba sp., for example from Entamoeba nuttalli. In yet another embodiment, the NADP-specific alcohol dehydrogenase has the amino acid sequence of SEQ ID NO: 45, is a variant of the amino acid sequence of SEQ ID NO: 45 or is a fragment of the amino acid sequence of SEQ ID NO: 45. In still another specific embodiment, the NADP-specific alcohol dehydrogenase is encoded by an heterologous nucleic acid molecule having the nucleic acid sequence of SEQ ID NO: 46, a variant of the nucleic acid sequence of SEQ ID NO: 46 or is a fragment of the nucleic acid sequence of SEQ ID NO: 46.

Alternatively or in combination, the yeast host cell can have a genetic modification allowing the expression of an heterologous saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

For example, the yeast host cell can bear one or more genetic modifications allowing for the production of an heterologous glucoamylase. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous protein is derived from a γ-amylase, such as, for example, the glucoamylase of Saccharomycoces filbuligera (e.g., encoded by the glu 0111 gene). Examples of yeast host cells bearing such second genetic modifications are described in WO 2011/153516 as well as in WO 2017/037614 and herewith incorporated in its entirety. In an embodiment, the yeast host cell can be modified to express an heterologous glucoamylase having the amino acid sequence of SEQ ID NO: 16, a variant thereof or a fragment thereof.

Alternatively or in combination, the yeast host cell can bear one or more genetic modifications for increasing formate/acetyl-CoA production. In order to do so, yeast host cell can bear one or more genetic modification for increasing its pyruvate formate lyase activity. As used in the context of the present disclosure, “an heterologous enzyme that function to increase formate/acetyl-CoA production” refers to polypeptides which may or may not be endogeneously found in the yeast host cell and that are purposefully introduced into the yeast host cells to anabolize formate. In some embodiments, the heterologous enzyme that can be an heterologous pyruvate formate lyase (PFL), such as PFLA or PFLB Heterologous PFL of the present disclosure include, but are not limited to, the PFLA polypeptide, a polypeptide encoded by a pfla gene ortholog, the PFLB polyeptide or a polypeptide encoded by a pflb gene ortholog.

Embodiments of the pyruvate formate lyase activating enzyme and of PFLA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli 0157: H7 str. Sakai (917728), Escherichia coli 083: H1 str. (12877392), Yersinia pestis (11742220), Clostridioides difficile (4915332), Vibrio fischeri (3278678), Vibrio parahaemolyticus (1188496), Vibrio coralliilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae 01 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli 0104: H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus malodoratus (34355146), Bacteroides oleiciplenus (32503668), Listeria (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfataricus (1454925), Streptococcus iniae (35765828), Streptococcus iniae (35764800), Bifidobacterium thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CHI (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063), Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostipes hadrus (31625694), Anaerostipes hadrus (31623667), Enterococcus haemoperoxidus (29838940), Gardnerella vaginalis (29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum (29475065), Bifidobacterium pseudolongum (34879480), Methanothermus fervidus (9962832), Methanothermus fervidus (9962056), Corynebacterium simulans (29536891), Thermoproteus uzoniensis (10359872), Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, the PFLA protein is derived from the genus Bifidobacterium and in some embodiments from the species Bifidobacterium adolescentis. In an embodiment, the yeast host cell expresses an heterologous PFLA polypeptide having the amino acid sequence of SEQ ID NO: 13, a variant thereof or a fragment thereof.

Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysenteriae (3795966), Bacillus thuringiensis serovar konkukian (2856201), Salmonella enterica subsp. enterica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. Steigerwaltii (34155981), Enterobacter hormaechei subsp. Steigerwaltii (34152298), Streptomyces venezuelae (34036549), Shewanella algae (34009243), Lactobacillus agilis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421), Salmonella enterica subsp. enterica serovar Typhi (1247405), Yersinia pestis (1174224), Yersinia enterocolitica subsp. enterocolitica (4713334), Streptococcus suis (8155093), Escherichia coli (947854), Escherichia coli (946315), Escherichia coli (945513), Escherichia coli (948904), Escherichia coli (917731), Yersinia enterocolitica subsp. enterocolitica (4714349), variants thereof as well as fragments thereof. In an embodiment, the PFLB protein is derived from the genus Bifidobacterium and in some embodiments from the specifies Bifidobacterium adolescentis. In such embodiments, the PFLB protein can have the amino acid sequence of SEQ ID NO: 7, be a variant of SEQ ID NO: 7 or be a fragment of SEQ ID NO: 7. In another embodiment, the recombinant yeast host cell comprises a nucleic acid molecule having the nucleic acid sequence of SEQ ID NO: 16 or 17. In an embodiment, the heterologous nucleic acid molecule encoding the PFLB protein is present in at least one, two, three, four, five or more copies in the recombinant yeast host cell. In still another embodiment, the heterologous nucleic acid molecule encoding the PFLB protein is present in no more than five, four, three, two or one copy/ies in the recombinant yeast host cell. The yeast host cell can be modified to express an heterologous PFLB polypeptide having the amino acid sequence of SEQ ID NO: 14, a variant thereof or a fragment thereof.

In some embodiments, the recombinant yeast host cell comprises a second genetic modification for expressing a PFLA protein, a PFLB protein or a combination. In a specific embodiment, the recombinant yeast host cell comprises a second genetic modification for expressing a PFLA protein and a PFLB protein which can, in some embodiments, be provided on distinct heterologous nucleic acid molecules. As indicated below, the recombinant yeast host cell can also include additional genetic modifications to provide or increase its ability to transform acetyl-CoA into an alcohol such as ethanol.

Alternatively or in combination, the yeast host cell can bear one or more genetic modifications for utilizing acetyl-CoA for example, by providing or increasing acetaldehyde and/or alcohol dehydrogenase activity. Acetyl-coA can be converted to an alcohol such as ethanol using second an acetaldehyde dehydrogenase and then an alcohol dehydrogenase. Acylating acetaldehyde dehydrogenases (E.C. 1.2.1.10) are known to catalyze the conversion of acetaldehyde into acetyl-CoA in the presence of CoA. Alcohol dehydrogenases (E.C. 1.1.1.1) are known to be able to catalyze the conversion of acetaldehyde into ethanol. The acetaldehyde dehydrogenase and alcohol dehydrogenase activity can be provided by a single protein (e.g., a bifunctional acetaldehyde/alcohol dehydrogenase) or by a combination of more than one protein (e.g., an acetaldehyde dehydrogenase and an alcohol dehydrogenase). In embodiments in which the acetaldehyde/alcohol dehydrogenase activity is provided by more than one protein, it may not be necessary to provide the combination of proteins in a recombinant form in the recombinant yeast host cell as the cell may have some pre-existing acetyldehyde or alcohol dehydrogenase activity. In such embodiments, the sixth genetic modification can include providing one or more heterologous nucleic acid molecule encoding one or more of an heterologous acetaldehyde dehydrogenase (AADH), an heterologous alcohol dehydrogenase (ADH) and/or heterologous bifunctional acetalaldehyde/alcohol dehydrogenases (ADHE). For example, the sixth genetic modification can comprise introducing an heterologous nucleic acid molecule encoding an acetaldehyde dehydrogenase. In another example, the sixth genetic modification can comprise introducing an heterologous nucleic acid molecule encoding an alcohol dehydrogenase. In still another example, the sixth genetic modification can comprise introducing at least two heterologous nucleic acid molecules, a second one encoding an heterologous acetaldehyde dehydrogenase and a second one encoding an heterologous alcohol dehydrogenase. In another embodiment, the sixth genetic modification comprises introducing an heterologous nucleic acid encoding an heterologous bifunctional acetaldehyde/alcohol dehydrogenases (AADH) such as those described in U.S. Pat. No. 8,956,851 and WO 2015/023989. Heterologous AADHs of the present disclosure include, but are not limited to, the ADHE polypeptides or a polypeptide encoded by an adhe gene ortholog. In an embodiment, the AADH has the amino acid sequence of SEQ ID NO: 15, is a variant of the amino acid sequence of SEQ ID NO: 15 or is a fragment of the amino acid sequence of SEQ ID NO: 15. In such embodiment, the genetic modification can comprise introducing an heterologous nucleic acid molecule encoding a protein having the amino acid sequence of SEQ ID NO: 15, being a variant of the amino acid sequence of SEQ ID NO: 15 or being a fragment of the amino acid sequence of SEQ ID NO: 15.

The yeast host cell described herein can be provided as a combination with the bacterial host cell described herein. In such combination, the yeast host cell can be provided in a distinct container from the bacterial host cell. The yeast host cell can be provided as a cell concentrate. The cell concentrate comprising the yeast host cell can be obtained, for example, by propagating the yeast host cells in a culture medium and removing at least one components of the medium comprising the propagated yeast host cell. This can be done, for example, by dehydrating, filtering (including ultra-filtrating) and/or centrifuging the medium comprising the propagated yeast host cell. In an embodiment, the yeast host cell is provided as a cream in the combination.

### Bacterial Host Cell

In the context of the present disclosure, the host cell is a bacterium and, in some embodiments, a lactic acid bacterium (LAB). As it is known in the art, LAB are a group of Gram-positive bacteria, non-respiring non-spore-forming, cocci or rods, which produce lactic acid as the major end product of the fermentation of carbohydrates. Bacterial genus of LAB include, but are not limited to, Lactobacillus, Leuconostoc, Pediococcus, Lactococcus, Streptococcus, Aerococcus, Carnobacterium, Enterococcus, Oenococcus, Sporolactobacillus, Tetragenococcus, Vagococcus, and Weissella. Bacterial species of LAB include, but are not limited to, Lactococcus lactis, Lactococcus garviae, Lactococcus raffinolactis, Lactococcus plantarum, Oenococcus oeni, Pediococcus pentosaceus, Pediococcus acidilactici, Carnococcus allantoicus, Carnobacterium gallinarum, Vagococcus fessus, Streptococcus thermophilus, Enterococcus phoeniculicola, Enterococcus plantarum, Enterococcus raffinosus, Enterococcus avium, Enterococcus pallens Enterococcus hermanniensis, Enterococcus faecalis, and Enterococcus faecium. In an embodiment, the LAB is a Lactobacillus and, in some additional embodiment, the Lactobacillus species is L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. brevis, L. buchneri, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii (including L. delbrueckii subsp. bulgaricus, L. delbrueckii subsp. delbrueckii, L. delbrueckii subsp. lactis), L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. ammesii, L. hamsteri, L. harbinensis, L. hayakitensis, L. helveticus, L. hilgardii, L. omohiochii, L. iners, L. ingluviei, L. intestinalis, L. jensenii, L. johnsonii, L. kalixensis, L. efiranofaciens, L. kefiri, L. kimchii, L. kitasatonis, L. kunkeei, L. leichmannii, L. lindneri, L. alefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. aralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae or L. zymae. In some embodiments, the bacterial host cell is L. paracasei and in some embodiments, L. paracasei 12A. For example, the bacterial host cell can be one of those described in WO 2018/013791.

The bacterial host cell of the present disclosure can have a second metabolic pathway comprising one or more second enzymes for producing a second metabolic product (from the first metabolic product). The bacterial host cell can have native enzymes present in the second metabolic pathway and be capable to produce the second metabolic product. Alternatively or in combination, the bacterial host cell can include one or more genetic modification to increase the activity of the one or more enzymes in the second metabolic pathway. The increased in activity is due at least in part to the introduction of one or more second genetic modifications in a native bacterial host cell to obtain the bacterial host cell. As such, the activity of the one or more second enzymes of the bacterial host cell is considered “increased” because it is higher than the activity of the one or more second enzymes in the native bacterial host cell (e.g., prior to the introduction of the one or more second genetic modifications). The one or more second genetic modifications is not limited to a specific modification provided that it does increase the activity, and in some embodiments, the expression of the one or more second enzymes. For example, the one or more second genetic modifications can include the addition of a promoter to increase the expression of the one or more (endogenous) second enzymes. Alternatively or in addition, the one or more second genetic modifications can include the introduction of one or more copies of a gene(s) encoding the one or more second (heterologous) enzymes in the bacterial host cell.

In the embodiment in which the first metabolic product is a carbohydrate such as trehalose, the second metabolic product can be ethanol and involve the anabolism of glucose-6-phosphate. In such embodiment, the bacterial host cell can have native activity in a PTS transporter, a trehalose-6-phosphate, an hexokinase and/or be genetically modified to provide or increase biological activity in at least one of a PTS transporter, a trehalose-6-phosphate or an hexokinase. In another embodiment in which the first metabolic product is a carbohydrate such as trehalose, the second metabolic product can be ethanol and involve the anabolism of acetaldehyde. In such embodiment, the bacterial host cell can have native pyruvate decarboxylase activity and/or be genetically modified to provide or increase pyruvate decarboxylase activity. In still another embodiment in which the first metabolic product is a carbohydrate such as trehalose, the second metabolic product can be ethanol. In such embodiment, the bacterial host cell can have native alcohol dehydrogenase activity and/or be genetically modified to provide or increase alcohol dehydrogenase activity. In an embodiment, the bacterial host cell has increased biological activity of a pyruvate decarboxylase, but not of the alcohol dehydrogenase. In another embodiment, the bacterial host cell has increased biological activity of an alcohol dehydrogenase, but not of the pyruvate decarboxylase. In still another embodiment, the bacterial host cell has increased biological activity in both a pyruvate decarboxylase and an alcohol dehydrogenase. As indicated above, this can be done by introducing a strong and/or constitutive promoter to increase the expression of the endogenous pyruvate decarboxylase and/or the endogenous alcohol dehydrogenase. Alternatively or in combination, this can also be done by introducing at least one copy of one or more heterologous nucleic acid molecules encoding an heterologous a pyruvate decarboxylase and/or an heterologous alcohol dehydrogenase.

In another embodiment in which the first metabolic product is an organic acid (or its associated esther), such as acetic acid (or acetate), the second metabolic product can be ethanol and involve the anabolism of the acetic acid (or acetate). As used in the context of the present disclosure, the expression “organic acid” includes associated organic esthers which can be hydrolyzed into the organic acid. In such embodiment, the bacterial host cell have native citrate lyase activity (to convert citric acid/citrate into acetic acid/acetate and oxaloacetate) and/or be genetically modified to provide or increase citrate lyase activity. Optionally, the bacterial host cell can have native pyruvate decarboxylase activity and/or be genetically modified to provide or increase pyruvate decarboxylase activity. Alternatively or in combination, the bacterial host cell can have native alcohol dehydrogenase activity and/or be genetically modified to provide or increase alcohol dehydrogenase activity. Alternatively or in combination, the bacterial host cell can have a native oxaloacetate decarboxylase and/or be genetically modified to provide or increase oxaloacetate decarboxylase activity. As indicated above, this can be done by introducing a strong and/or constitutive promoter to increase the expression of the endogenous citrate lyase, the endogenous pyruvate decarboxylase, the endogenous alcohol dehydrogenase and/or the endogenous oxaloacetate decarboxylase Alternatively or in combination, this can also be done by introducing at least one copy of one or more heterologous nucleic acid molecules encoding an heterologous citrate lyse, an heterologous a pyruvate decarboxylase, an heterologous alcohol dehydrogenase and/or an heterologous oxaloacetate decarboxylase.

As used herein, the term “citrate lyase” refers to an enzyme catalyzing the conversion of citrate into acetate and oxaloacetate (EC 4.1.3.6). In some embodiments, the citrate lyase is obtained from a Lactobacillus sp., such as for example, a Lactobacillus paracasei. In such embodiment, the citrate lyase can have the amino acid sequence of SEQ ID NO: 17, be a variant of the amino acid sequence of SEQ ID NO: 17 or be a fragment of the amino acid of SEQ ID NO: 17 or a variant thereof. Still in additional embodiments, the citrate lyase can be encoded by an heterologous nucleic acid molecule having the nucleic acid sequence of SEQ ID NO: 18, a variant of the nucleic acid sequence of SEQ ID NO: 18 or a fragment of the nucleic acid sequence of SEQ ID NO: 18 or variant thereof. In some embodiments, the citrate lyase can comprise the beta chain of the citrate lyase of a Lactobacillus sp., such as for example, a Lactobacillus paracasei. In such embodiment, the beta chain of the citrate lyase can have the amino acid sequence of SEQ ID NO: 19, be a variant of the amino acid sequence of SEQ ID NO: 19 or be a fragment of the amino acid of SEQ ID NO: 19 or a variant thereof. Still in additional embodiments, the beta chain of the citrate lyase can be encoded by an heterologous nucleic acid molecule having the nucleic acid sequence of SEQ ID NO: 20, a variant of the nucleic acid sequence of SEQ ID NO: 20 or a fragment of the nucleic acid sequence of SEQ ID NO: 20 or variant thereof. In some embodiments, the citrate lyase can comprise the gamma chain of the citrate lyase of a Lactobacillus sp., such as for example, a Lactobacillus paracasei. In such embodiment, the gamma chain of the citrate lyase can have the amino acid sequence of SEQ ID NO: 21, be a variant of the amino acid sequence of SEQ ID NO: 21 or be a fragment of the amino acid of SEQ ID NO: 21 or a variant thereof. Still in additional embodiments, the gamma chain of the citrate lyase can be encoded by an heterologous nucleic acid molecule having the nucleic acid sequence of SEQ ID NO: 22, a variant of the nucleic acid sequence of SEQ ID NO: 22 or a fragment of the nucleic acid sequence of SEQ ID NO: 22 or variant thereof.

As used herein, the term “oxaloacetate decarboxylase” refers to an enzyme catalyzing the decarboxylation of oxaloacetate to pyruvate and carbon dioxide (E.C. 4.1.1.3). In some embodiments, the oxaloacetate decarboxylase is obtained from a Lactobacillus sp., such as for example, a Lactobacillus paracasei. In such embodiment, the oxaloacetate decarboxylase can have an alpha chain comprising the amino acid sequence of SEQ ID NO: 23, be a variant of the amino acid sequence of SEQ ID NO: 23 or be a fragment of the amino acid of SEQ ID NO: 23 or a variant thereof. Still in additional embodiments, the alpha chain of the oxaloacetate decarboxylase can be encoded by an heterologous nucleic acid molecule having the nucleic acid sequence of SEQ ID NO: 24, a variant of the nucleic acid sequence of SEQ ID NO: 24 or a fragment of the nucleic acid sequence of SEQ ID NO: 24 or variant thereof. In some embodiments, the oxaloacetate decarboxylase can comprise a beta chain of obtained from a Lactobacillus sp., such as for example, a Lactobacillus paracasei. In such embodiment, the beta chain of the oxaloacetate decarboxylase can have the amino acid sequence of SEQ ID NO: 25, be a variant of the amino acid sequence of SEQ ID NO: 25 or be a fragment of the amino acid of SEQ ID NO: 25 or a variant thereof. Still in additional embodiments, the beta chain of the oxaloacetate decarboxylase can be encoded by an heterologous nucleic acid molecule having the nucleic acid sequence of SEQ ID NO: 26, a variant of the nucleic acid sequence of SEQ ID NO: 26 or a fragment of the nucleic acid sequence of SEQ ID NO: 26 or variant thereof. In some embodiments, the oxaloacetate decarboxylase can comprise a gamma chain of obtained from a Lactobacillus sp., such as for example, a Lactobacillus paracasei. In such embodiment, the gamma chain of the oxaloacetate decarboxylase can have the amino acid sequence of SEQ ID NO: 55, be a variant of the amino acid sequence of SEQ ID NO: 55 or be a fragment of the amino acid of SEQ ID NO: 55 or a variant thereof. Still in additional embodiments, the gamma chain of the oxaloacetate decarboxylase can be encoded by an heterologous nucleic acid molecule having the nucleic acid sequence of SEQ ID NO: 56, a variant of the nucleic acid sequence of SEQ ID NO: 56 or a fragment of the nucleic acid sequence of SEQ ID NO: 56 or variant thereof. In some additional embodiments, the oxaloacetate decarboxylase is a trimeric polypeptide comprises at least one of an alpha chain (having the amino acid sequence of SEQ ID NO: 23, a variant thereof or a fragment thereof), a beta chain (having the amino acid sequence of SEQ ID NO: 25, a variant thereof or a fragment thereof) or a gamma chain (having the amino acid sequence of SEQ ID NO: 55, a variant thereof or a fragment thereof). In some additional embodiments, the oxaloacetate decarboxylase is a trimeric polypeptide comprises at least two of an alpha chain (having the amino acid sequence of SEQ ID NO: 23, a variant thereof or a fragment thereof), a beta chain (having the amino acid sequence of SEQ ID NO: 25, a variant thereof or a fragment thereof) or a gamma chain (having the amino acid sequence of SEQ ID NO: 55, a variant thereof or a fragment thereof). In some additional embodiments, the oxaloacetate decarboxylase is a trimeric polypeptide comprises an alpha chain (having the amino acid sequence of SEQ ID NO: 23, a variant thereof or a fragment thereof), a beta chain (having the amino acid sequence of SEQ ID NO: 25, a variant thereof or a fragment thereof) and a gamma chain (having the amino acid sequence of SEQ ID NO: 55, a variant thereof or a fragment thereof).

As used herein, the term “pyruvate decarboxylase” refers to an enzyme catalyzing the decarboxylation of pyruvic acid to acetaldehyde and carbon dioxide. In Zymonas mobilis, the pyruvate decarboxylase gene is referred to as PDC (Gene ID: 33073732) and could be used in the bacterial host cell of the present disclosure. In some additional embodiments, the pyruvate decarboxylase polypeptide can be from Lactobacillus florum (Accession Number WP_009166425.1), Lactobacillus fructivorans (Accession Number WP_039145143.1), Lactobacillus lindneri (Accession Number WP_065866149.1), Lactococcus lactis (Accession Number WP_104141789.1), Carnobacterium gallinarum (Accession Number WP_034563038.1), Enterococcus plantarum (Accession Number WP_069654378.1), Clostridium acetobutylicum (Accession Number NP_149189.1), Bacillus megaterium (Accession Number WP_075420723.1) or Bacillus thuringiensis (Accession Number WP_052587756.1). In the bacterial host cell of the present disclosure, the pyruvate decarboxylase can have the amino acid of SEQ ID NO: 4, be a variant of SEQ ID NO: 4 or a fragment of SEQ ID NO: 4. In some specific embodiments, the bacterial host cell of the present disclosure can express an heterologous nucleic acid molecule comprising the nucleic acid sequence of any one of SEQ ID NO: 1 to 3.

As used herein, the term “alcohol dehydrogenase” refers to an enzyme of the EC 1.1.1.1 class. In some embodiments, the alcohol dehydrogenase is an iron-containing alcohol dehydrogenase. The alcohol dehydrogenase that can be expressed in the bacterial host cell includes, but is not limited to, ADH4 from Saccharomyces cerevisiae, ADHB from Zymonas mobilis, FUCO from Escherichia coli, ADHE from Escherichia coli, ADH1 from Clostridium acetobutylicum, ADH1 from Entamoeba nuttalli, BDHA from Clostridium acetobutylicum, BDHB from Clostridium acetobutylicum, 4HBD from Clostridium kluyveri, DHAT from Citrobacter freundii or DHAT from Klebsiella pneumoniae. In an embodiment, the alcohol dehydrogenase can be ADHB from Zymonas mobilis (Gene ID: AHJ71151.1), Lactobacillus reuteri (Accession Number: KRK51011.1), Lactobacillus mucosae (Accession Number WP_048345394.1), Lactobacillus brevis (Accession Number WP_003553163.1) or Streptococcus thermophiles (Accession Number WP_113870363.1). In the bacterial host cell of the present disclosure, the pyruvate decarboxylase can have the amino acid of SEQ ID NO: 8, be a variant of SEQ ID NO: 8 or a fragment of SEQ ID NO: 8. In some specific embodiments, the bacterial host cell of the present disclosure can express an heterologous nucleic acid molecule comprising the nucleic acid sequence of any one of SEQ ID NO: 5 to 7.

In a specific embodiment, the recombinant yeast host cell can express an heterologous polypeptide having NADPH-dependent alcohol dehydrogenase activity. The protein having NADPH-dependent alcohol dehydrogenase activity can be an ADH polypeptide (for example from Entamoeba sp., including Entamoeba nuttalli (such as, for example, the one having the amino acid sequence of SEQ ID NO: 45), an ADH1 polypeptide variant, an ADH1 polypeptide fragment or a polypeptide encoded by an ADH1 gene ortholog/paralog. In some specific embodiments, the bacterial host cell of the present disclosure can express an heterologous nucleic acid molecule comprising the nucleic acid sequence of SEQ ID NO: 46. In yet another embodiment, the heterologous gene coding for the NADPH-dependent alcohol dehydrogenase protein is present in one, two, three, four or more copies in the recombinant microbial host cell. In the embodiments in which the first metabolic product is a sugar alcohol such as mannitol, the second metabolic product can be ethanol and involve the anabolism of fructose-6-phosphate.

In such embodiment, the bacterial host cell can be selected for its ability to utilize mannitol because it comprises a native mannitol utilization operon. In such embodiment, it is possible to use the bacterial host cell without introducing a genetic modification to allow mannitol utilization. Alternatively or in combination, the bacterial host cell can have increased biological activity in one or more proteins encoded by the genes of the mannitol utilization operon. For example, the bacterial host cell can have increase biological activity in a mannitol-1-phophatase 5-dehydrogenase (such as MTLD2) and/or a mannitol transporter. In an embodiment, the MTLD2 polypeptide can be from Lactobacillus sp., such as, for example Lactobacillus casei. In some embodiments, the MTLD2 polypeptide can have the amino acid sequence of SEQ ID NO: 39, be a variant of the amino acid sequence of SEQ ID NO: 39 or be a fragment of the amino acid sequence of SEQ ID NO: 39 or a variant thereof. In some additional embodiments, the MTLD2 polypeptide can be encoded by an heterologous nucleic acid molecule having the nucleic acid sequence of SEQ ID NO: 40, a variant of the nucleic acid sequence of SEQ ID NO: 40 or a fragment of the nucleic acid sequence of SEQ ID NO: 40 or a fragment thereof. In an embodiment, the MTLCB polypeptide can be from Lactobacillus sp., such as, for example Lactobacillus casei. In some embodiments, the MTLCB polypeptide can have the amino acid sequence of SEQ ID NO: 41, be a variant of the amino acid sequence of SEQ ID NO: 41 or be a fragment of the amino acid sequence of SEQ ID NO: 41 or a variant thereof. In some additional embodiments, the MTLCB polypeptide can be encoded by an heterologous nucleic acid molecule having the nucleic acid sequence of SEQ ID NO: 42, a variant of the nucleic acid sequence of SEQ ID NO: 42 or a fragment of the nucleic acid sequence of SEQ ID NO: 42 or a fragment thereof. In an embodiment, the MTLA polypeptide can be from Lactobacillus sp., such as, for example Lactobacillus casei. In some embodiments, the MTLA polypeptide can have the amino acid sequence of SEQ ID NO: 43, be a variant of the amino acid sequence of SEQ ID NO: 43 or be a fragment of the amino acid sequence of SEQ ID NO: 43 or a variant thereof. In some additional embodiments, the MTLA polypeptide can be encoded by an heterologous nucleic acid molecule having the nucleic acid sequence of SEQ ID NO: 44, a variant of the nucleic acid sequence of SEQ ID NO: 44 or a fragment of the nucleic acid sequence of SEQ ID NO: 44 or a fragment thereof.

In the embodiments in which the first metabolic product is a sugar alcohol such as sorbitol, the second metabolic product can be ethanol and involve the anabolism of fructose-6-phosphate. In such embodiment, the bacterial host cell can be selected for its ability to utilize sorbitol because it comprises a native sorbitol utilization operon. In such embodiment, it is possible to use the bacterial host cell without introducing a genetic modification to allow sorbitol utilization. Alternatively or in combination, the bacterial host cell can have increased biological activity in one or more protein encoded by the genes of the sorbitol utilization operon. For example, the bacterial host cell can have increase biological activity in one or more proteins of the sorbitol operon which includes the gutF (encoding a sorbitol-6-phosphate dehydrogenase or the GUTF polypeptide), gutC (encoding the transporter subunit C or the GUTC polypeptide), gutB (encoding the transporter subunit B or the GUTB polypeptide) and gutA (encoding the transporter subunit A or the GUTA polypeptide) genes. In an embodiment, the GUTF polypeptide is from Lactobacillus sp., such as, for example Lactobacillus paracasei. In such embodiment, the GUTF polypeptide can have, for example, the amino acid sequence of SEQ ID NO: 31, be a variant of the amino acid sequence of SEQ ID NO: 31 or be a fragment of the amino acid sequence of SEQ ID NO: 31 or a variant thereof. In an embodiment, the GUTF polypeptide is encoded by an heterologous nucleic acid molecule having the nucleic acid sequence of SEQ ID NO: 32, being a variant of the nucleic acid sequence of SEQ ID NO: 32 or being a fragment of the nucleic acid sequence or SEQ ID NO: 32 or a variant thereof. In an embodiment, the GUTC polypeptide is from Lactobacillus sp., such as, for example Lactobacillus paracasei. In such embodiment, the GUTC polypeptide can have, for example, the amino acid sequence of SEQ ID NO: 33, be a variant of the amino acid sequence of SEQ ID NO: 33 or be a fragment of the amino acid sequence of SEQ ID NO: 33 or a variant thereof. In an embodiment, the GUTC polypeptide is encoded by an heterologous nucleic acid molecule having the nucleic acid sequence of SEQ ID NO: 34, being a variant of the nucleic acid sequence of SEQ ID NO: 34 or being a fragment of the nucleic acid sequence or SEQ ID NO: 34 or a variant thereof. In an embodiment, the GUTB polypeptide is from Lactobacillus sp., such as, for example Lactobacillus paracasei. In such embodiment, the GUTB polypeptide can have, for example, the amino acid sequence of SEQ ID NO: 35, be a variant of the amino acid sequence of SEQ ID NO: 35 or be a fragment of the amino acid sequence of SEQ ID NO: 35 or a variant thereof. In an embodiment, the GUTB polypeptide is encoded by an heterologous nucleic acid molecule having the nucleic acid sequence of SEQ ID NO: 36, being a variant of the nucleic acid sequence of SEQ ID NO: 36 or being a fragment of the nucleic acid sequence or SEQ ID NO: 36 or a variant thereof. In an embodiment, the GUTA polypeptide is from Lactobacillus sp., such as, for example Lactobacillus paracasei. In such embodiment, the GUTA polypeptide can have, for example, the amino acid sequence of SEQ ID NO: 37, be a variant of the amino acid sequence of SEQ ID NO: 37 or be a fragment of the amino acid sequence of SEQ ID NO: 37 or a variant thereof. In an embodiment, the GUTA polypeptide is encoded by an heterologous nucleic acid molecule having the nucleic acid sequence of SEQ ID NO: 38, being a variant of the nucleic acid sequence of SEQ ID NO: 38 or being a fragment of the nucleic acid sequence or SEQ ID NO: 38 or a variant thereof.

In the embodiments in which the first metabolic product is a sugar alcohol such as glycerol, the second metabolic product can be ethanol and involved the anabolism of dihydroxyacetone-phosphate. The bacterial host cell can have native or engineered activity in a second metabolic pathway, e.g., the glycerol dehydrogenase/DHA kinase pathway. In such embodiment, the bacterial host cell comprises native or engineered increased biological activity in one or more of a glycerol hydrogenase and/or dihydroxyacetone kinase. Alternatively or in combination, the bacterial host cell can have native or engineered activity in another second metabolic pathway, e.g., the glycerol kinase/glycerol-3-phosphate dehydrogenase pathway. In such embodiment, the bacterial host cell comprises native or engineered increased biological activity in one or more of a glycerol kinase and/or a glycerol-3-phosphate dehydrogenase. Alternatively or in combination, the bacterial host cell can have a native and/or be genetically modified to provide or increase a glycerol facilitator activity.

In some embodiments, the bacterial host cell can be further modified to inactivate one or more endogenous genes. In the context of the present disclosure, the inactivation of a gene refers to the removal of at least one nucleic acid residue so as to impede the expression of the endogenous genes. The at least one nucleic acid residue can be removed in the coding or the non-coding region of the gene. In some embodiments, the entire coding region of a gene is removed to inactivate the gene. In some additional embodiments, one or more additional nucleic acid residues can be added at the location at which the deletion occurred.

In a specific embodiment, especially when the trehalose or acetic acid/acetate is the first metabolic product, the bacterial host cell can be modified to as to decrease is lactate dehydrogenase activity. As used in the context of the present disclosure, the expression “lactate dehydrogenase” refer to an enzyme of the E.C. 1.1.1.27 class which is capable of catalyzing the conversion of pyruvic acid into lactate. The bacterial host cells can thus have one or more gene coding for a protein having lactate dehydrogenase activity which is inactivated (via partial or total deletion of the gene). In bacteria, the ldh1, ldh2, ldh3 and ldh4 genes encode proteins having lactate dehydrogenase activity. Some bacteria may contain as many as six or more such genes (i.e., ldh5, ldh6, etc.) In an embodiment, at least one of the ldh1, ldh2, ldh3 and ldh4 genes, their corresponding orthologs and paralogs is inactivated in the bacterial host cell. In an embodiment, only one of the ldh genes is inactivated in the bacterial host cell. For example, in the bacterial host cell of the present disclosure, only the ldh1 gene can be inactivated. In another embodiment, at least two of the ldh genes are inactivated in the bacterial host cell. In another embodiment, only two of the Idh genes are inactivated in the bacterial host cell. In a further embodiment, at least three of the Idh genes are inactivated in the bacterial host cell. In a further embodiment, only three of the ldh genes are inactivated in the bacterial host cell. In a further embodiment, at least four of the Idh genes are inactivated in the bacterial host cell. In a further embodiment, only four of the Idh genes are inactivated in the bacterial host cell. In a further embodiment, at least five of the Idh genes are inactivated in the bacterial host cell. In a further embodiment, only five of the ldh genes are inactivated in the bacterial host cell. In a further embodiment, at least six of the ldh genes are inactivated in the bacterial host cell. In a further embodiment, only six of the ldh genes are inactivated in the bacterial host cell. In still another embodiment, all of the Idh genes are inactivated in the bacterial host cell.

In a specific embodiment, especially when trehalose or acetic acid/acetate is the first metabolic product, the bacterial host cell can be modified so as to decrease its mannitol-1-phosphate 5-dehydrogenase activity. As used in the context of the present disclosure, the expression “mannitol-1-P 5-dehydrogenase” refer to an enzyme of the E.C. 1.1.1.17 class which is capable of catalyzing the conversion of mannitol into fructose-6-phosphate. The bacterial host cells can thus have one or more gene coding for a protein having mannitol dehydrogenase activity which is inactivated (via partial or total deletion of the gene). In bacteria, the mltd1 and mltd2 genes encode proteins having mannitol-1-P 5-dehydrogenase activity. In an embodiment, at least one of the mltd1 and mtld2 genes, their corresponding orthologs and paralogs is inactivated in the bacterial host cell. In an embodiment, only one of the mltd1 and mtld2 genes is inactivated in the bacterial host cell. In another embodiment, both of the mltd1 and mtld2 genes are inactivated in the bacterial host cell.

The bacterial host cell described herein can be provided as a combination with the yeast cell described herein. In such combination, the bacterial host cell can be provided in a distinct container from the yeast cell. The bacterial host cell can be provided as a cell concentrate. The cell concentrate comprising the bacterial host cell can be obtained, for example, by propagating the bacterial host cells in a culture medium and removing at least one components of the medium comprising the propagated bacterial host cell. This can be done, for example, by dehydrating, filtering (including ultra-filtrating) and/or centrifuging the medium comprising the propagated bacterial host cell. In an embodiment, the bacterial host cell is provided as a frozen concentrate in the combination.

### Process of Using the Yeast Host Cell and the Bacterial Host Cell

The combination of the host cells described herein can be used to improve alcohol (e.g., ethanol) yield in a fermentation. As shown herein, some embodiments the combination of the yeast host cells and of the bacterial host cells are advantageous as they improve the robustness of the yeast host cells in the presence of a stressor during fermentation. The stressor can be, for example, a bacterial contamination, an increase in pH, a reduction in aeration, elevated temperatures, osmotic pressure or combinations thereof. In some embodiments, the process described herein can also be used to limit glucose and/or glycerol concentration during fermentation. In some other embodiments, the process described herein can also be used to limit or prevent contamination of the fermentation by other non-fermenting microorganisms (especially when the bacterial yeast host cell is capable of producing one or more bacteriocin).

The biomass that can be fermented with the combination of host cells described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line-rich proteins). In some embodiments, the biomass can include and/or be supplemented with citric acid (especially when acetic acid or acetate is the first metabolic product).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The process of the present disclosure contacting the host cells described herein with a biomass so as to allow the conversion of at least a part of the biomass into the fermentation product. The fermented product can be an alcohol, such as, for example, ethanol, isopropanol, n-propanol, 1-butanol, methanol, acetone and/or 1, 2 propanediol. In an embodiment, the biomass or substrate to be hydrolyzed is a lignocellulosic biomass and, in some embodiments, it comprises starch (in a gelatinized or raw form). In the process of the present disclosure, the yeast host cells can be second contacted with the biomass. Alternatively, the bacterial host cells can be second contacted with the biomass. Also, in some embodiments, both the yeast host cells and the bacterial host cells can be contacted simultaneously with the biomass.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### Example I—Trehalose Utilization

Expression cassettes for trehalose-6-P synthase (TPS1, SEQ ID NO: 9) and trehalose-6-P phosphatase (TPS2, SEQ ID NO: 10) from Saccharomyces cerevisiae were engineered into strain S. cerevisiae strain M12156 which contains glycerol reduction technology and expresses a glucoamylase. The cassettes were integrated at the IME1 locus, in a knock in fashion. The CYC1 terminator sequence was included downstream of the IME1 open reading frame (ORF) followed by the TPS1 and TPS2 expression cassettes which were driven by the promoters of TDH1 and PAU5 respectively. TDH1 is predicted to give strong constitutive expression of TPS1 whereas the PAU5 promoter has been shown to be induced by alcoholic fermentation and anaerobic conditions. The resulting strain was given the identifier M16807. The table below summarizes the genotype of the Saccharomyces cerevisiae strains used in this example.

TABLE 1
Genotype of Saccharomyces cerevisiae strains
used in this example.

| | | Gene(s) |
|---|---|---|
| Strain | Gene(s) overexpressed | inactivated |
| M12156 | STL1 (SEQ ID NO: 11) | fdh1Δ |
| | ADHE (SEQ ID NO: 15) | fdh2Δ |
| | PFLA (SEQ ID NO: 13) | gpd2Δ |
| | PFLB (SEQ ID NO: 14) | |
| | GLU (SEQ ID NO: 16) | |
| M16807 | Same as M12156 | Same as M12156 |
| | TPS1 (SEQ ID NO: 9) | |
| | TPS2 (SEQ ID NO: 10) | |

The Lactobacillus paracasei strain 12A was engineered into an ethanologen by deletion of four native LDH enzymes coupled with the addition of the PDC (SEQ ID NO: 4) and ADHB (SEQ ID NO: 8 encoded by codon-optimized SEQ ID NO: 6 and 7) enzymes from Z. mobilis. Two copies of the Z. mobilis genes (codon-optimized SEQ ID NO: 2 and 3) were integrated into the genome with one cassette driven by the glycolytic pgm promoter, and the second cassette driven by the promoter of the universal stress protein A (uspA) which has been shown to be up-regulated during late growth stages. In addition two native genes encoding mannitol-1-phosphate 5-dehydrogenase, mtlD1 and mtlD2, were also deleted to eliminate the conversion of fructose-6-phosphate to mannitol. The genotype of strain Lactobacillus paracasei used in this example is provided in Table 2.

TABLE 2
Genotype of Lactobacillus paracasei strain used
in this example.

| | | Gene(s) |
|---|---|---|
| Strain | Gene(s) overexpressed | inactivated |
| 12A | None - wild-type Lactobacillus | |
| | paracasei parental strain | |
| M17744 | PDC (SEQ ID NO: 4) | ldh1Δ, ldh2Δ, ldh3Δ, |
| (E3.1) | ADHB (SEQ ID NO: 8) | ldh4Δ mtlD1Δ, mtlD2Δ |

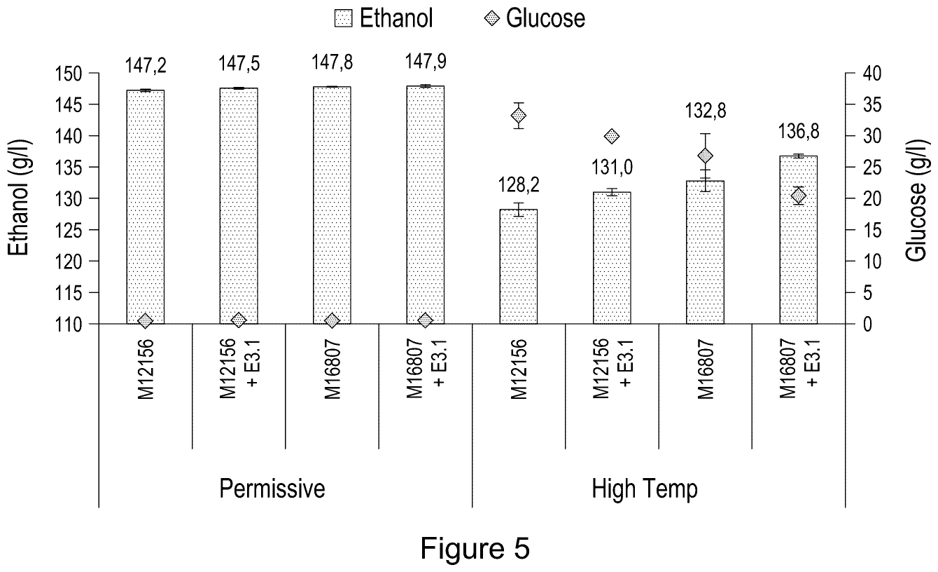

S. cerevisiae strains M12156 and M16807 were utilized to ferment commercial corn mash either with or without the inclusion of strain E3.1. Performance was characterized under standard commercial operating parameters (permissive) as well in the presence of high temperature stress. Fermentation parameters are outlined in Table 3 and metabolite concentrations were analyzed by HPLC following 50 hours of fermentation. As shown on FIG. 5, the results indicated that both M12156 and M16807 perform similarly under standard conditions either with or without the addition of E3.1. Conversely, when the strains underwent high temperature stress, M16807 produced significantly more ethanol than strain M12156 and had lower residual glucose at the end of fermentation. Likewise, co-fermentation with the ethanologen E3.1 also showed improved results for both M12156 and M16807 under stressful conditions. Most significantly, the combination of the new yeast strain M16807 with E3.1 had a synergistic effect showing higher ethanol titers than would be expected from the additive effects of trehalose biosynthesis and co-fermentation with E3.1.

TABLE 3
Fermentation parameters utilized to analyze performance
in corn mash fermentation.

| | | M12156 + | | M16807 + |
|---|---|---|---|---|
| | M12156 | E3.1 | M16807 | E3.1 |
| Yeast Dose gDCW/L | 0.3 | 0.3 | 0.3 | 0.3 |
| Bacterial Dose cfu/ml | N/A | 1 × 10\hat{ }7 | N/A | 1 × 10\hat{ }7 |
| % Total Solids | 31.50% | 31.50% | 31.50% | 31.50% |
| Spirizyme Excel GA Dose | 0.42 | 0.42 | 0.42 | 0.42 |
| (AGU/gTS) | | | | |
| Urea ppm | 300 | 300 | 300 | 300 |
| Temperature 0-24 hours | 33° C. | 33° C. | 35° C. | 35° C. |
| Temperature 24-50 hours | 31° C. | 31° C. | 33° C. | 33° C. |

### Example II—Mannitol and Sorbitol Utilization

The sorbitol constructs included Saccharomyces cerevisiae M20043, which was constructed by introducing 4-copies (2-per chromosome) of the E. coli srlD, encoding sorbitol-6-phophate dehydrogenase, into the fcyl locus of wild-type strain M2390. The corresponding engineered bacterium was Lactobacillus paracasei M19605, which was constructed from the ethanologen strain E3 (ΔL-ldh1::P_{pgm}-PET, ΔL-Idh2, ΔD-hic, ΔmtlD1, ΔmtlD2, ΔL-ldh3PuspA-PET) by introduction of plasmid pDW2::P₃₁-gutFCBA, which encode the sorbitol-6-phosphate dehydrogenase, and transporter subunits C, B, and A respectively.

The mannitol constructs were Saccharomyces cerevisiae M20036, which was engineered from M2390 by introducing 4-copies (two per chromosome) of the Escherichia coli mtlD, encoding mannitol-1-phosphate 5-dehydrogenase. The corresponding bacterium for this fermentation was Lactobacillus paracasei M19998, which was constructed from the ethanologen strain E3.1 (ΔL-ldh1::P_{pgm}-PET, ΔL-ldh2, ΔD-hic, ΔmtlD1, ΔmtlD2, ΔL-ldh3PuspA-PET, ΔL-Idh4) by introduction of plasmid pDW2::P₃₁-mtIDCBA, which encode the mannitol-1-phosphate 5-dehydrogenase and transporter subunits C/B and A respectively.

Tables 4 and 5 summarize the genotypes of the yeast and bacterial host cells used in this Example.

TABLE 4
Genotype of Saccharomyces cerevisiae strains
used in this example.

| | | Gene(s) |
|---|---|---|
| Strain | Gene(s) overexpressed | inactivated |
| M2390 | None - wild type parental strain used | |
| | for M20043 and M20036 | |
| M20043 | SRLD (SEQ ID NO: 29) | fcyΔ |
| M20036 | MTLD (SEQ ID NO: 35) | |

TABLE 5
Genotype of Lactobacillus paracasei strain
used in this example.

| | | Gene(s) |
|---|---|---|
| Strain | Gene(s) overexpressed | inactivated |
| M19605 | PDC (SEQ ID NO: 4) | ldh1Δ, ldh2Δ, ldh3Δ, |
| | ADHB (SEQ ID NO: 8) | ldh4Δ mtlD1Δ, mtlD2Δ |
| | GUTF (SEQ ID NO: 31) | |
| | GUTC (SEQ ID NO: 33) | |
| | GUTB (SEQ ID NO: 35) | |
| | GUTA (SEQ ID NO: 37) | |
| M19998 | PDC (SEQ ID NO: 4) | ldh1Δ, ldh2Δ, ldh3Δ, |
| | ADHB (SEQ ID NO: 8) | ldh4Δ mtlD1Δ, mtlD2Δ |
| | MTLD (SEQ ID NO: 27) | |
| | MTLCB (SEQ ID NO: 41) | |
| | MTLA (SEQ ID NO: 43) | |

The engineered yeast and bacteria were grown individually or in combination in a modified chemically defined medium (mCDM) that contained the following components (per L): 2.0 g sodium citrate (2 H₂O), 1.0 g Potassium phosphate (mono basic), 1.0 g potassium phosphate (di basic), 200 mg sodium chloride, 200 mg calcium chloride (2 H₂O), 200 mg magnesium sulfate, 50 mg manganese sulfate, 1 mL Tween 80™, 1 mL Tween 20™, 1 mL glycerol, 10 μL mevalonolactone, 10 mg pyridoxal HCl, 20.0 mL RPMI 1640 vitamin solution, 10.0 g Bacto-casitone, 2.5 mg pyridoxamine dihydrochloride and 18 g Glucose (100 mM). All of the cell samples were washed twice with 0.85% saline, normalized to an OD₆₀₀ of 2.0 and inoculated at 0.1%. Samples were incubated at 35° C. for 67 hours, then the supernatant was collected and analyzed by HPLC.

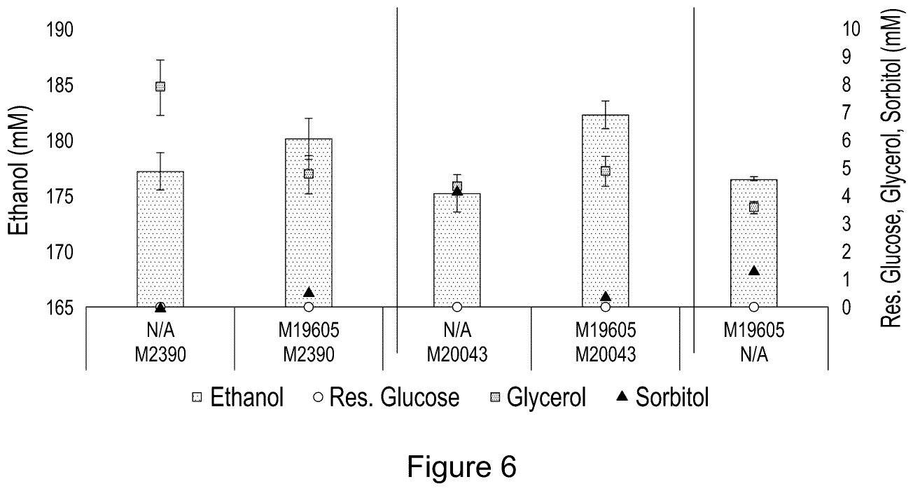

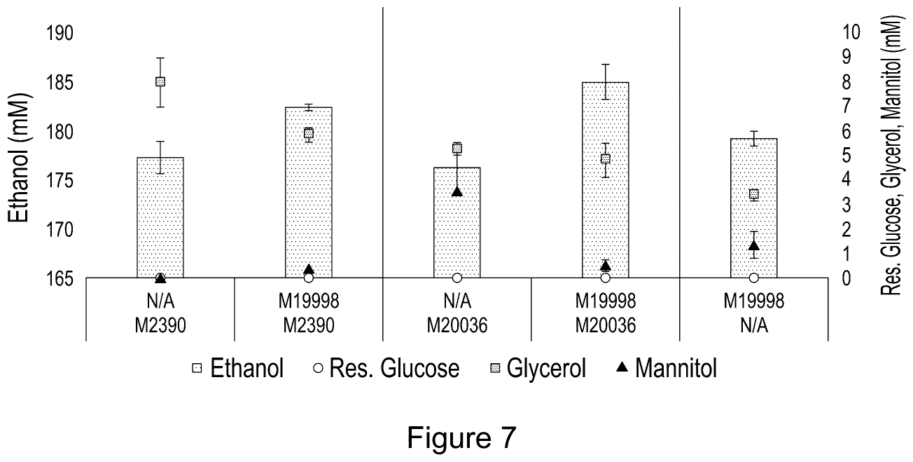

As shown in FIGS. 6 and 7 as well as Table 6, the wild-type control strain of Saccharomyces cerevisiae (M2390) converted the glucose into 177.2 mM ethanol and 7.9 mM glycerol. As expected, fermentation of mCDM with the engineered yeast strains M20043 or M20036 alone led to reduced glycerol titers and slightly lower ethanol levels, as carbon was redirected from glycerol biosynthesis toward sorbitol or mannitol, respectively, in these hosts. Strain M20043 produced 4.2 mM sorbitol and decreased glycerol production by 45% compared to the wild-type yeast M2390. The mannitol-producing yeast M20036 accumulated 3.5 mM mannitol in the fermentate, and reduced glycerol levels by 35% compared to M2390 (Table 6).

Growth in mCDM by pure cultures of Lactobacillus paracasei ethanologens engineered to convert sorbitol (M19605) or mannitol (M19998) into ethanol contained lower levels of glycerol than was observed with individual yeast strains, and yielded ethanol levels that were similar to or slightly above results from single yeast (FIGS. 6 and 7 as well as Table 6).

In contrast, fermentations that were performed with yeast and bacteria pairs uniformly showed increased ethanol levels, even with the wild-type control yeast strain, M2390 (FIGS. 6 and 7 as well as Table 6). Co-fermentation with the sorbitol producing yeast M20043 and the sorbitol consuming bacterium M19605 enhanced ethanol yield by 2.9% over M2390 alone, compared to 1.6% when the bacterium was paired with M2390. As expected, the sorbitol observed in fermentations with M20043 alone was largely consumed when the yeast was paired with M19605. These data demonstrate the added yield obtained with M20043 and M19605 is the result of metabolic redirection of glycerol biosynthesis to ethanol (via sorbitol) by the co-engineered yeast and bacterium.

Co-fermentations with the mannitol producing yeast M200363 and the mannitol consuming bacterium M19998 showed a similar pattern. Ethanol production in the fermentation with co-engineered yeast and bacteria was 4.4% higher than M2390 alone, whereas a 2.8% increase was obtained when M19998 was paired with wild-type M2390. Once again, the mannitol that was present in fermentations with M20036 alone was essentially consumed when the yeast was paired with M19998. These data demonstrate the added yield obtained with M20036 and M19998 is the result of metabolic redirection of glycerol biosynthesis to ethanol (via mannitol) by the co-engineered yeast and bacterium.

TABLE 6
Final metabolite concentrations in mCDM fermented with yeast and
bacteria strains co-engineered to redirect glycerol biosynthesis to ethanol.

| | | Metabolite concentration (mM) | | | | |
|---|---|---|---|---|---|---|
| Strain | | Glucose | Glycerol | Sorbitol | Mannitol | Ethanol |
| S. cerevisiase | M2390 | 0 | 7.9 | 0 | 0 | 177.2 |
| S. cerevisiase | M20043 | 0 | 4.3 | 4.2 | 0 | 175.3 |
| S. cerevisiase | M20036 | 0 | 5.2 | 0 | 3.5 | 176.1 |
| L. paracasei | M19605 | 0 | 3.6 | 1.3 | 0 | 176.6 |
| L. paracasei | M19998 | 0 | 3.4 | 0 | 1.3 | 179.2 |
| M2390 + | M19605 | 0 | 4.8 | 0.5 | 0 | 180.2 |
| M20043 + | M19605 | 0 | 4.9 | 0.4 | 0 | 182.3 |
| M2390 + | M19998 | 0 | 5.8 | 0 | 0.4 | 182.3 |
| M20036 + | M19998 | 0 | 4.8 | 0 | 0.5 | 185.0 |

### Example III—Acetate Utilization

Wild type strain Saccharomyces cerevisiae M8279 was engineered for acetate utilization by introducing 4-copies (2-per chromosome) of the Bifidobacterium adolescentis adhE and up-regulation of the ACS2 polypeptide (e.g., additional copies of the native gene (SEQ ID NO: 49) were included), encoding a bi-functional acetaldehyde/alcohol dehydrogenase and an acetyl-CoA synthetase respectively, at the ylr296W locus. In addition, 4-copies (2-per chromosome) of the heterologous NADP-specific alcohol dehydrogenase of Entamoeba nuttalli (e.g., having the amino acid sequence of SEQ ID NO: 45) was integrated at the apt2 locus. The presence of this enzyme increases the availability of cytosolic NADH, by creating a redox imbalance between glycolysis and ethanol fermentation, and increases acetate conversion in S. cerevisiae. As the introduced acetate conversion pathway is required to compete for NADH with the native glycerol biosynthetic pathway, the later was down regulated by deletion of gpd2, encoding a glycerol-3-phosphate dehydrogenase, and up-regulation of an heterologous glycerol transporter STL1 (from P. sorbitophila) resulting in the final yeast strain M10909.

TABLE 7
Genotype of Saccharomyces cerevisiae strains
used in this example.

| | | Gene(s) |
|---|---|---|
| Strain | Gene(s) overexpressed | inactivated |
| M8279 | None - wild-type Saccharomyces | |
| | cerevisiae parental strain | |
| M10909 | STL1 (SEQ ID NO: 51) | apt2Δ |
| | ADHE (SEQ ID NO: 15) | gpd2Δ |
| | ACS2 (SEQ ID NO: 49) | |
| | NADP-specific alcohol | |
| | dehydrogenase of | |
| | Entamoeba nuttalli | |
| | (SEQ ID NO: 45) | |

The engineered bacterium, M20896, is derived from the Lactobacillus paracasei strain 12A, which was converted to an ethanologen through deletion of four native lactate dehydrogenases, two native mannitol dehydrogenases, and incorporation of a heterologous production of ethanol cassette (PET) consisting of the Zymomonas mobilis pyruvate decarboxylase, and alcohol dehydrogenase (ΔL-Idh1::Ppgm-PET, ΔL-Idh2, ΔD-hic, ΔmtlD1, ΔmtlD2, ΔL-Idh3PuspA-PET). No additional modifications were therefore made to the native citrate operon.

TABLE 8
Genotype of Lactobacillus paracasei strain
used in this example.

| | Gene(s) | |
|---|---|---|
| Strain | overexpressed | Gene(s) inactivated |
| 12A | None - wild-type Lactobacillus | |
| | paracasei parental strain | |
| M20896 | PDC (SEQ ID NO: 4) | ldh1Δ, ldh2Δ, ldh3Δ, |
| | ADHB (SEQ ID NO: 8) | ldh4Δ mtlD1Δ, mtlD2Δ |
| E5 | PDC (SEQ ID NO: 4) | ldh1Δ, ldh2Δ, ldh3Δ, |
| | ADHB (SEQ ID NO: 8) | ldh4Δ mtlD1Δ, mtlD2Δ |

The engineered yeast and bacteria were grown individually or in combination in a modified chemically defined medium (mCDM) that contained either 50 or 100 mM glucose (e.g., for 1 L of mCDM: 2.0 g sodium citrate (2 H₂O), 1.0 g potassium phosphate (mono basic), 1.0 g potassium phosphate (di basic), 200 mg sodium chloride, 200 mg calcium chloride (2 H₂O), 200 mg magnesium sulfate, 50 mg manganese sulfate, 1 mL Tween™ 80, 1 mL Tween™ 20, 1 mL glycerol, 10 μL mevalonolactone, 10 mg pyridoxal HCl, 20.0 mL RPMI 1640 vitamin solution, 10.0 g bacto-casitone, 2.5 mg pyridoxamine dihydrochloride and 18 g glucose (100 mM) or 9 g Glucose (50 mM)). When indicated, sodium citrate was removed from the media preparation in order to determine the impact of citrate conversion on fermentation performance. The wild type yeast strain M8279 was also included in these experiments. All of the cell samples were washed 2× with 0.85% saline, normalized to an OD₆₀₀ of 2.0 and inoculated at 0.1%. Samples were incubated at 35° C. for 68 hours, then the supernatant was collected and analyzed by HPLC.

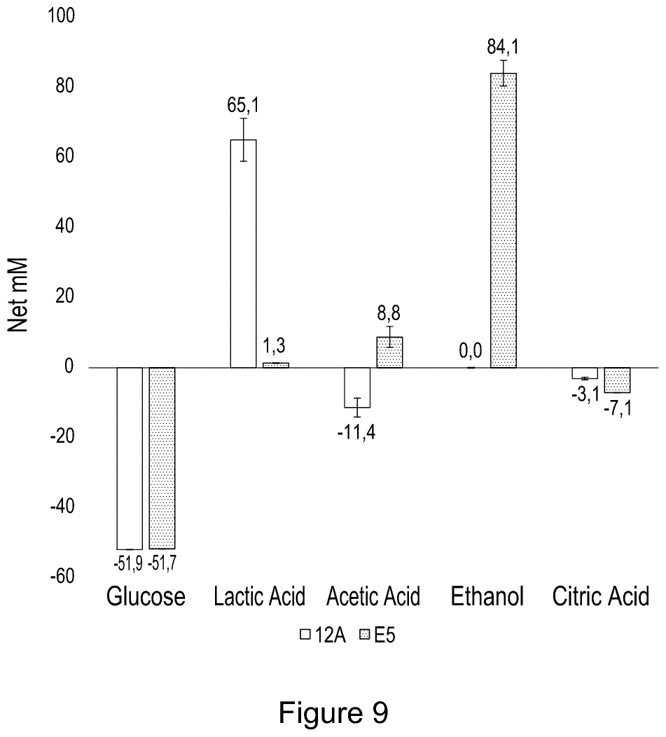

As shown in FIG. 9, the wild-type control strain 12A only consumed approximately 40% of available citrate when grown in mCDM (50 mM glucose) and consumed 11 mM of acetate. Conversely, E5, an ethanologen strain containing equivalent ethanol engineering as M20896 and differing only in their antimicrobial resistance profile, completely depleted citrate and generated acetate as a result (FIG. 9).

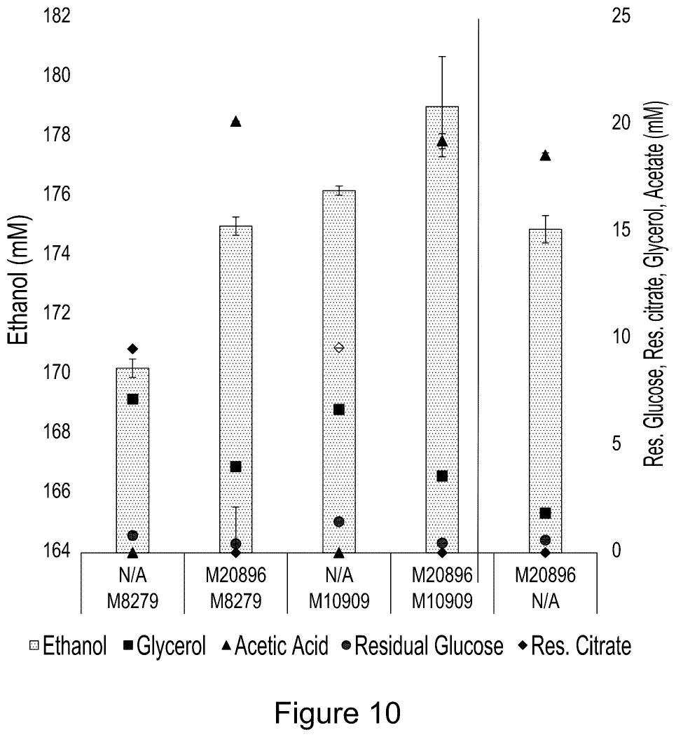

As shown in FIG. 10 and Table 9, the wild-type control strain of Saccharomyces cerevisiae (M8279) converted the glucose into 170.2 mM ethanol and 7.2 mM glycerol. As expected, fermentation of mCDM with the engineered yeast strains M10909 alone led to reduced glycerol titers and higher ethanol levels, as carbon was redirected from glycerol biosynthesis due to the down regulation of this pathway. Strain M10909 produced 6.7 mM glycerol and increased ethanol yield by 4.2% compared to the wild-type yeast M8279 (Table 9). Similarly, it was observed that co-fermentation with M20896 and M8279 led to a 2.4% yield increase over M8279 alone and a 35% reduction in glycerol titer.

TABLE 9
Final metabolite concentrations in mCDM fermented with yeast and
bacteria strains co-engineered to convert citrate/acetate to ethanol

| | | Metabolite concentration (mM) | | | | |
|---|---|---|---|---|---|---|
| Strain | | Glucose | Glycerol | Acetate | Citrate | Ethanol |
| S. cerevisiae | M8279¹ | 0.80 | 7.2 | 0.0 | 9.5 | 170.2 |
| S. cerevisiae | M10909² | 1.44 | 6.7 | 0.0 | 9.6 | 176.2 |
| Lb. paracasei | M20896³ | 0.58 | 1.8 | 18.6 | 0.0 | 174.9 |
| M8279 + | M20896 | 0.42 | 4.0 | 20.2 | 0.0 | 175.0 |
| M10909 + | M20896 | 0.44 | 3.6 | 19.3 | 0.0 | 179.0 |

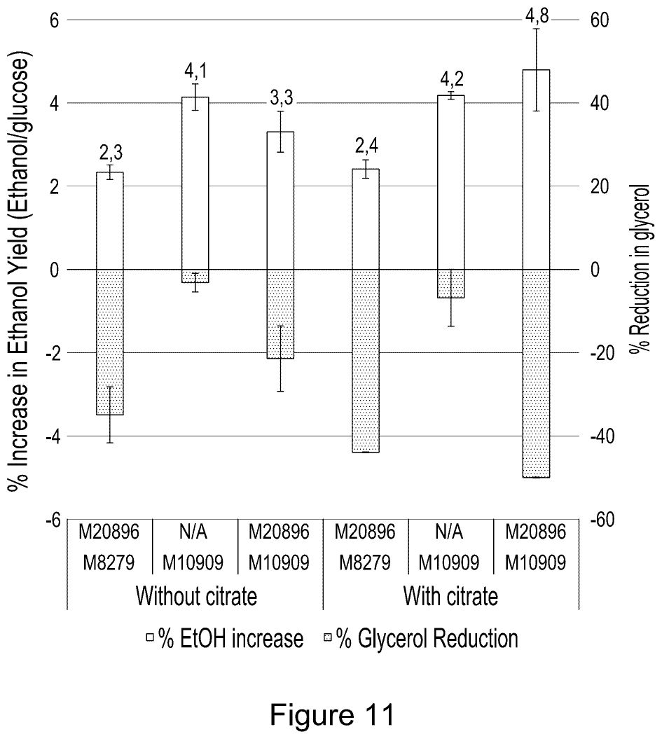

In contrast, when co-fermentations were performed utilizing both the engineered yeast and the bacterium pair, an overall ethanol yield increase was seen of 4.8% and a 50% reduction in glycerol titer was achieved (FIG. 11). This corresponded to a 3 mM increase in ethanol titer over M10909 alone while 1 mM of acetate was consumed.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Peleg, A.Y., Hogan, D.A., Mylonakis, E., 2010. Medically important bacterial-fungal interactions. Nat. Rev. Microbiol. 8, 340-349.
Schink, B., 2002. Synergistic interactions in the microbial world. Antonie Van Leeuwenhoek 81, 257-261.
Wargo, M.J., Hogan, D. A., 2006. Fungal-bacterial interactions: a mixed bag of mingling microbes. Curr. Opin. Microbiol., Host microbe interactions: fungi/Host microbe interactions: parasites/Host microbe interactions: viruses 9, 359-364.
Yi, C., Wang, F., Dong, S., Li, H. 2016. Changes of trehalose content and expression of relative genes during the bioethanol fermentation by Saccharomyces cerevisiae. Can. J. Microbiol. 62:827-835.
U.S. Pat. No. 8,956,851
WO 2012/138942
WO 2011/153516
WO 2017/037614
WO 2015/023989
WO 2018/013791

1. A combination of a first microbial host cell having a first metabolic pathway comprising one or more first enzymes for producing a first metabolic product and a second microbial host cell having a second metabolic pathway comprising one or more second enzymes for converting at least in part the first metabolic product into a second metabolic product, wherein:
at least one of the first microbial host cell or the second microbial host cell is recombinant;
at least one of the first microbial host cell or the second microbial host cell is a bacterial host cell;
at least one of the first microbial host cell or the second microbial host cell is a yeast host cell;
when the first microbial host cell is a recombinant first microbial host cell, the recombinant first microbial host cell has increased activity in the first metabolic pathway, when compared to a corresponding native first microbial host cell, for producing the first metabolic product; and
when the second microbial host cell is a recombinant second microbial host cell, the recombinant second microbial host cell has increased activity in the second metabolic pathway, when compared to a corresponding native second microbial host cell, for converting at least in part the first metabolic product into the second metabolic product.

2. The combination of claim 1, wherein the first microbial host cell is a bacterial host cell and the second microbial cell is a yeast host cell.

3. The combination of claim 2, wherein at least one of the one or more first enzymes are native enzymes and/or at least one of the one or more second enzymes are heterologous enzymes.

4. The combination of claim 2, wherein the first metabolic product is an organic acid or an ester thereof and/or the second metabolic product is ethanol and wherein:
the one or more first enzymes comprises a citrate lyase; and/or
the one or more second enzymes comprise one or more of:
one or more heterologous polypeptides having acetaldehyde dehydrogenase activity, and/or one or more heterologous polypeptides having acetyl-coA synthetase activity.

5. The combination of claim 4, wherein (i) the yeast host cell is the recombinant yeast host cell and (ii) the heterologous polypeptide having acetaldehyde dehydrogenase activity is an acetylating dehydrogenase (AADH) or a bifunctional acetaldehyde/alcohol dehydrogenase (ADHE), the one or more second enzymes comprising an heterologous polypeptide having NADP⁺-dependent alcohol dehydrogenase activity and/or an heterologous polypeptide having acetyl-coA synthetase activity.

6. The combination of claim 1, wherein the first microbial host cell is a yeast host cell and the second microbial host cell is a bacterial host cell.

7. The combination of claim 6, wherein at least one of the one or more first enzymes is a heterologous enzyme and/or at least one of the one or more second enzymes is a heterologous enzyme.

8. The combination of claim 6, wherein the first metabolic product is a carbohydrate and/or the second metabolic product is ethanol.

9. The combination of claim 8, wherein (i) the carbohydrate is trehalose and (ii) the one or more first enzymes comprises trehalose-6-phosphate synthase and/or trehalose-6-phosphate phosphatase.

10. The combination of claim 8, wherein (i) the carbohydrate is mannitol, and (ii) the one or more first enzymes comprises mannitol-1-phosphate 5-dehydrogenase, the one or more second enzymes comprise a product of at least one gene from a mannitol utilization operon, and/or the one or more second enzymes comprises a mannitol transporter.

11. The combination of claim 8, wherein (i) the carbohydrate is sorbitol and (ii) the one or more first enzymes comprises sorbitol-6-phosphate dehydrogenase, and/or the one or more second enzymes comprises a product of at least one gene from a sorbitol utilization operon.

12. The combination of claim 8, wherein (i) the carbohydrate is glycerol, (ii) the one or more second enzymes comprise at least one of a glycerol dehydrogenase, a dihydroxyacetone kinase, a glycerol kinase, a glycerol-3-phosphate dehydrogenase, and/or a glycerol facilitator.

13. The combination of claim 12, wherein the yeast host cell has increased activity, when compared to the corresponding native yeast host cell, in an NADP⁺-dependent aldehyde dehydrogenase and/or in a phosphoketolase.

14. The combination of claim 1, wherein the yeast host cell is from Saccharomyces sp. or from Saccharomyces cerevisiae.

15. The combination of claim 1, wherein the bacterial host cell further comprises a third metabolic pathway comprising one or more third enzymes for producing a third metabolic product.

16. The combination of claim 15, wherein the third metabolic product is ethanol and the one or more third enzymes for producing the third metabolic product comprises a pyruvate decarboxylase and/or an alcohol dehydrogenase; and/or wherein the bacterial host cell has a decreased lactate dehydrogenase activity when compared to the corresponding native bacterial host cell.

17. The combination of claim 1, wherein the bacterial host cell is a lactic acid bacteria.

18. The combination of claim 17, wherein the bacterial host cell is from Lactobacillus sp. or from Lactobacillus paracasei.

19. A process for converting a biomass into a fermentation product, the process comprises contacting the biomass with the combination of claim 1 under condition to allow conversion of at least a part of the biomass into the fermentation product.

20. A commercial package comprising:
(i) a combination of a first microbial host cell having a first metabolic pathway comprising one or more first enzymes for producing a first metabolic product and a second microbial host cell having a second metabolic pathway comprising one or more second enzymes for converting at least in part the first metabolic product into a second metabolic product, wherein:
at least one of the first microbial host cell or the second microbial host cell is recombinant;
at least one of the first microbial host cell or the second microbial host cell is a bacterial host cell;
at least one of the first microbial host cell or the second microbial host cell is a yeast host cell;
when the first microbial host cell is a recombinant first microbial host cell, the recombinant first microbial host cell has increased activity in the first metabolic pathway, when compared to a corresponding native first microbial host cell, for producing the first metabolic product; and
when the second microbial host cell is a recombinant second microbial host cell, the recombinant second microbial host cell has increased activity in the second metabolic pathway, when compared to a corresponding native second microbial host cell, for converting at least in part the first metabolic product into the second metabolic product; and
(ii) instructions to perform a process for converting a biomass into a fermentation product, the process comprises contacting the biomass with the combination of (i) under condition to allow conversion of at least a part of the biomass into the fermentation product.
